SEQUENCE LISTING (31 sequences)

SEQ ID NO: 1 - protein, 451 aa, Artificial Sequence
  SYNTHETIC CONSTRUCT
MNLEGGGRGGEFGMSAVSCGNGKLRQWLIDQIDSGKYPGLVWENEEKSIFRIPWKHAGKQDYNREEDAALFKAWALFKGKFREGIDKPDPPTWKTRLRCALNKSNDFEELVERSQLDISDPYKVYRIVPEGAKKGAKQLTLEDPQMSMSHPYTMTTPYPSLPAQQVHNYMMPPLDRSWRDYVPDQPHPEIPYQCPMTFGPRGHHWQGPACENGCQVTGTFYACAPPESQAPGVPTEPSIRSAEALAFSDCRLHICLYYREILVKELTTSSPEGCRISHGHTYDASNLDQVLFPYPEDNGQRKNIEKLLSHLERGVVLWMAPDGLYAKRLCQSRIYWDGPLALCNDRPNKLERDQTCKLFDTQQFLSELQAFAHHGRSLPRFQVTLCFGEEFPDPQRQRKLITAHVEPLLARQLYYFAQQNSGHFLRGYDLPEHISNPEDYHRSIRHSSIQE

SEQ ID NO: 2 - DNA, 1356 bp, Artificial Sequence
  SYNTHETIC CONSTRUCT
atgaacctggagggcggcggccgaggcggagagttcggcatgagcgcggtgagctgcggcaacgggaagctccgccagtggctgatcgaccagatcgacagcggcaagtaccccgggctggtgtgggagaacgaggagaagagcatcttccgcatcccctggaagcacgcgggcaagcaggactacaaccgcgaggaggacgccgcgctcttcaaggcttgggcactgtttaaaggaaagttccgagaaggcatcgacaagccggaccctcccacctggaagacgcgcctgcggtgcgctttgaacaagagcaatgactttgaggaactggttgagcggagccagctggacatctcagacccgtacaaagtgtacaggattgttcctgagggagccaaaaaaggagccaagcagctcaccctggaggacccgcagatgtccatgagccacccctacaccatgacaacgccttacccttcgctcccagcccagcaggttcacaactacatgatgccacccctcgaccgaagctggagggactacgtcccggatcagccacacccggaaatcccgtaccaatgtcccatgacgtttggaccccgcggccaccactggcaaggcccagcttgtgaaaatggttgccaggtgacaggaaccttttatgcttgtgccccacctgagtcccaggctcccggagtccccacagagccaagcataaggtctgccgaagccttggcgttctcagactgccggctgcacatctgcctgtactaccgggaaatcctcgtgaaggagctgaccacgtccagccccgagggctgccggatctcccatggacatacgtatgacgccagcaacctggaccaggtcctgttcccctacccagaggacaatggccagaggaaaaacattgagaagctgctgagccacctggagaggggcgtggtcctctggatggcccccgacgggctctatgcgaaaagactgtgccagagcaggatctactgggacgggcccctggcgctgtgcaacgaccggcccaacaaactggagagagaccagacctgcaagctctttgacacacagcagttcttgtcagagctgcaagcgtttgctcaccacggccgctccctgccaagattccaggtgactctatgctttggagaggagtttccagaccctcagaggcaaagaaagctcatcacagctcacgtagaacctctgctagccagacaactatattattttgctcaacaaaacagtggacatttcctgaggggctacgatttaccagaacacatcagcaatccagaagattaccacagatctatccgccattcctctattcaagaatga

SEQ ID NO: 3 - DNA, 2024 bp, Artificial Sequence
  SYNTHETIC CONSTRUCT
gaggctgtgtgggttctatcatgtgtattggggattgcgtgtgtgttaagagatagcattacgtaggactctattggggaatagagtgtgttagagatctcatatctgtatctgaagatagcatgagccctgaggattgtgtgtgtttattgcaggatagtatatgtgtttgagattgtgtgggctctatcatgtgtgctggggattgcatgtgtgtggctattggcatagggaaagtaattagggctaccccagcaacagccttgctcctcaccacactgctagggaggaaaggagaaagtaaggaccagaagaaggtacagcagaaggggaaaagagaaagataggcagaggtcctttcactctccacggaaagatattccagtacctgcaggcctgaggtgttgtggaaagcaatagggatgtgctgacagcctgctgctcgggctcctgctcctctccagtcctcatgatctgctctgtatagtctgagtcccataggaagattctagatagaacatcagagcaaggcagggtggaatctgtccagaattcacaataggctcatagccagtatctaatcacctagccttgacaggggtcttgctaccctgagcatgccagaaagacataaaggtataaaggaggctctgtaacagccaggcgtggggaggggatccccctagcttctgcccacaggccccatgctgagactggaggcggccagtctgtgcctcacactctttttccatttctgtgctgttcaaagtaatgtttccttccccaccaagctagtgcctctgaacctgggtggctgaggcagttgccagatggtttccaggcgggctgcctattttagggtggtgagacccacacatctcattgctaatattagcagtttcgttttccctttttttcccattcactgtggcgaaacacagagagcagaatgattaaatcatcagaatgccccagaaatgactagccagccaggtactatgcatgcacacaagtcggcccaccccacctaatcccagagagacaagcaggaggtgaggtgggcctccacctcctagggactgaggaaagttgactgggaaagacctagaaattgagtctaccccagcctggtgttaggtttttctcaggggaagagaaagatgcagggcagcagagctagcaaacctaagacaactatagaagcagagaaaacagtgagatccgggcagcagatccagcatcttgaaaggaagaaaagccttaagagaaagcaagaccaggcccagggtctttctgaacctacaggggtgtctggagaggaaaggcatcgtctcgggtcctaggaaatgttcactatagcccttcgaggcctcctctgacccatcaaacgggagcatgtgggatagctgggctcttgctcctcagtagtaaaggactaaggcatagctcagggcattcaaggccacgcatggcagacaaggtaggggagggtccagcttgccctcgctgcggccataggtaccaaagccaggcctcgacacccaccctccaaagggacaagagtctggccctagtttcagtctctctcagccctgggagctaaggctcgatcggggtaccaggaatggaaaagaccaaacctacccacaagaggggctagaaatggagaggaccccatagcaggacaagaggcaaggacagctagtcagagagaaccccccctctctgctccccaatctctcactagtcccttacctgctcctcccaggcatcgttccctcccactcccctcccccttccatgcccctcccccacctctagttgcctgttctcccacccttgtggaggtggaggaagagggggcgggagccaagaacaggtctcctccctccaacatgacctgggacagtttcctttccgctacagacaactctgcctgagcagcggggaggaggaagaggagactgctactgaaggcgacactgccaggggctctgggcatgtgggtccggcaggtaccctgg

SEQ ID NO: 4 - DNA, 22 bp, Artificial Sequence
  SYNTHETIC CONSTRUCT
aggaccagaagaaggtacagca

SEQ ID NO: 5 - DNA, 22 bp, Artificial Sequence
  SYNTHETIC CONSTRUCT
ttgctttccacaacacctcagg

SEQ ID NO: 6 - DNA, 24 bp, Artificial Sequence
  SYNTHETIC CONSTRUCT
agacccacacatctcattgctaat

SEQ ID NO: 7 - DNA, 22 bp, Artificial Sequence
  SYNTHETIC CONSTRUCT
cctggctggctagtcatttctg

SEQ ID NO: 8 - DNA, 22 bp, Artificial Sequence
  SYNTHETIC CONSTRUCT
gcagcagatccagcatcttgaa

SEQ ID NO: 9 - DNA, 21 bp, Artificial Sequence
  SYNTHETIC CONSTRUCT
tgcctttcctctccagacacc

SEQ ID NO: 10 - DNA, 21 bp, Artificial Sequence
  SYNTHETIC CONSTRUCT
ccaccctccaaagggacaaga

SEQ ID NO: 11 - DNA, 22 bp, Artificial Sequence
  SYNTHETIC CONSTRUCT
ccctcttgtgggtaggtttggt

SEQ ID NO: 12 - DNA, 24 bp, Artificial Sequence
  SYNTHETIC CONSTRUCT
caagaacaggtctcctccctccaa

SEQ ID NO: 13 - DNA, 23 bp, Artificial Sequence
  SYNTHETIC CONSTRUCT
agtgtcgccttcagtagcagtct

SEQ ID NO: 14 - DNA, 34 bp, Artificial Sequence
  SYNTHETIC CONSTRUCT
catactcttaacagtcagaatgaaacctgggtgg

SEQ ID NO: 15 - DNA, 20 bp, Artificial Sequence
  SYNTHETIC CONSTRUCT
gcgcaaggtaagaactgtcc

SEQ ID NO: 16 - DNA, 20 bp, Artificial Sequence
  SYNTHETIC CONSTRUCT
acagccctccaaatgtcaac

SEQ ID NO: 17 - DNA, 21 bp, Artificial Sequence
  SYNTHETIC CONSTRUCT
agggtgagcaggcgagagcaa

SEQ ID NO: 18 - DNA, 23 bp, Artificial Sequence
  SYNTHETIC CONSTRUCT
agcacagggaagaagctgttggg

SEQ ID NO: 19 - DNA, 22 bp, Artificial Sequence
  SYNTHETIC CONSTRUCT
cctcacctcctgcttgtctctc

SEQ ID NO: 20 - DNA, 23 bp, Artificial Sequence
  SYNTHETIC CONSTRUCT
gtgagacccacacatctcattgc

SEQ ID NO: 21 - DNA, 23 bp, Artificial Sequence
  SYNTHETIC CONSTRUCT
ctctgactagctgtccttgcctc

SEQ ID NO: 22 - DNA, 21 bp, Artificial Sequence
  SYNTHETIC CONSTRUCT
ctcgacacccaccctccaaag

SEQ ID NO: 23 - DNA, 20 bp, Artificial Sequence
  SYNTHETIC CONSTRUCT
ggaggggataggcgctgggt

SEQ ID NO: 24 - DNA, 20 bp, Artificial Sequence
  SYNTHETIC CONSTRUCT
tctgggccaagccatccggt

SEQ ID NO: 25 - DNA, 5323 bp, Artificial Sequence
  SYNTHETIC CONSTRUCT
acctcgcactctcagtttcaccgctcgatcttgggacccaccgctgccctcagctccgagtccagggcgagtgcagagcagagcgggcggaggaccccgggcgcgggcgcggacggcacgcgggcatgaacctggagggcggcggccgaggcggagagttcggcatgagcgcggtgagctgcggcaacgggaagctccgccagtggctgatcgaccagatcgacagcggcaagtaccccgggctggtgtgggagaacgaggagaagagcatcttccgcatcccctggaagcacgcgggcaagcaggactacaaccgcgaggaggacgccgcgctcttcaaggcttgggcactgtttaaaggaaagttccgagaaggcatcgacaagccggaccctcccacctggaagacgcgcctgcggtgcgctttgaacaagagcaatgactttgaggaactggttgagcggagccagctggacatctcagacccgtacaaagtgtacaggattgttcctgagggagccaaaaaaggagccaagcagctcaccctggaggacccgcagatgtccatgagccacccctacaccatgacaacgccttacccttcgctcccagcccagcaggttcacaactacatgatgccacccctcgaccgaagctggagggactacgtcccggatcagccacacccggaaatcccgtaccaatgtcccatgacgtttggaccccgcggccaccactggcaaggcccagcttgtgaaaatggttgccaggtgacaggaaccttttatgcttgtgccccacctgagtcccaggctcccggagtccccacagagccaagcataaggtctgccgaagccttggcgttctcagactgccggctgcacatctgcctgtactaccgggaaatcctcgtgaaggagctgaccacgtccagccccgagggctgccggatctcccatggacatacgtatgacgccagcaacctggaccaggtcctgttcccctacccagaggacaatggccagaggaaaaacattgagaagctgctgagccacctggagaggggcgtggtcctctggatggcccccgacgggctctatgcgaaaagactgtgccagagcaggatctactgggacgggcccctggcgctgtgcaacgaccggcccaacaaactggagagagaccagacctgcaagctctttgacacacagcagttcttgtcagagctgcaagcgtttgctcaccacggccgctccctgccaagattccaggtgactctatgctttggagaggagtttccagaccctcagaggcaaagaaagctcatcacagctcacgtagaacctctgctagccagacaactatattattttgctcaacaaaacagtggacatttcctgaggggctacgatttaccagaacacatcagcaatccagaagattaccacagatctatccgccattcctctattcaagaatgaaaaatgtcaagatgagtggttttctttttccttttttttttttttttttgatacggggatacggggtcttgctctgtctcccaggctggagtgcagtgacacaatctcagctcactgtgacctccgcctcctgggttcaagagactctcctgcctcagcctccctggtagctgggattacaggtgtgagccactgcacccacccaagacaagtgattttcattgtaaatatttgactttagtgaaagcgtccaattgactgccctcttactgttttgaggaactcagaagtggagatttcagttcagcggttgaggagaattgcggcgagacaagcatggaaaatcagtgacatctgattggcagatgagcttatttcaaaaggaagggtggctttgcatttcttgtgttctatagactgccatcattgatgatcactgtgaaaattgaccaagtgatgtgtttacatttactgaaatgtgctctttaatttgttgtagattaggtcttgctggaagacagagaaaacttgcctttcagtattgacactgactagagtgatgactgcttgtaggtatgtctgtgccatttctcagggaagtaagatgtaaattgaagaagcctcacacgtaaaagaaatgtattaatgtatgtaggagctgcagttcttgtggaagacacttgctgagtgaaggaaatgaatctttgactgaagccgtgcctgtagccttggggaggcccatcccccacctgccagcggtttcctggtgtgggtccctctgccccaccctccttcccattggctttctctccttggcctttcctggaagccagttagtaaacttcctattttcttgagtcaaaaaacatgagcgctactcttggatgggacatttttgtctgtcctacaatctagtaatgtctaagtaatggttaagttttcttgtttctgcatctttttgaccctcattctttagagatgctaaaattcttcgcataaagaagaagaaattaaggaacataaatcttaatacttgaactgttgcccttctgtccaagtacttaactatctgttcccttcctctgtgccacgctcctctgtttgcttggctgtccagcgatcagccatggcgacactaaaggaggaggagccggggactcccaggctggagagcactgccaggacccaccactggaagcaggatggagctgactacggaactgcacactcagtgggctgtttctgcttatttcatctgttctatgcttcctcgtgccaattatagtttgacagggccttaaaattacttggctttttccaaatgcttctatttatagaatcccaaagacctccacttgcttaagtatacctatcacttacatttttgtggttttgagaaagtacagcagtagactggggcgtcacctccaggccgtttctcatactacaggatatttactattactcccaggatcagcagaagattgcgtagctctcaaatgtgtgttcctgcttttctaatggatattttaaattcattcaacaagcacctagtaagtgcctgctgtatccctacattacacagttcagcctttatcaagcttagtgagcagtgagcactgaaacattattttttaatgtttaaaaagtttctaatattaaagtcagaatattaatacaattaatattaatattaactacagaaaagacaaacagtagagaacagcaaaaaaataaaaaggatctccttttttcccagcccaaattctcctctctaaaagtgtccacaagaaggggtgtttattcttccaacacatttcacttttctgtaaatatacataaacttaaaaagaaaacctcatggagtcatcttgcacacactttcatgcagtgctctttgtagctaacagtgaagatttacctcgttctgctcagaggccttgctgtggagctccactgccatgtacccagtagggtttgacatttcattagccatgcaacatggatatgtattgggcagcagactgtgtttcgtgaactgcagtgatgtatacatcttatagatgcaaagtattttggggtatattatcctaagggaagataaagatgatattaagaactgctgtttcacggggcccttacctgtgaccctctttgctgaagaatatttaaccccacacagcacttcaaagaagctgtcttggaagtctgtctcaggagcaccctgtcttcttaattctccaagcggatgctccatttcaattgctttgtgacttcttcttctttgtttttttaaatattatgctgctttaacagtggagctgaattttctggaaaatgcttcttggctggggccactacctcctttcctatctttacatctatgtgtatgttgactttttaaaattctgagtgatccagggtatgacctagggaatgaactagctatgaaatactcagggttaggaatcctagcacttgtctcaggactctgaaaaggaacggcttcctcattccttgtcttgataaagtggaattggcaaactagaatttagtttgtactcagtggacagtgctgttgaagatttgaggacttgttaaagagcactgggtcatatggaaaaaatgtatgtgtctcccaggtgcatttcttggtttatgtcttgttcttgagattttgtatatttaggaaaacctcaagcagtaattaatatctcctggaacactatagagaaccaagtgaccgactcatttacaactgaaacctaggaagcccctgagtcctgagcgaaaacaggagagttagtcgccctacaggaaacccagctagactattgggtatgaactaaaaagagactgtgccatggtgagaaaaatgtaaaatcctacagtggaatgagcagcccttacagtgttgttaccaccaagggcaggtaggtattagtgtttgaaaaagctggtctttgagcgagggcataaatacagctagccccaggggtggaacaactgtgggagtcttgggtactcgcacctcttggctttgttgatgctccgccaggaaggccacttgtgtgtgcgtgtcagttacttttttagtaacaattcagatccagtgtaaacttccgttcattgctctccagtcacatgcccccacttccccacaggtgaaagtttttctgaaagtgttgggattggttaaggtctttatttgtattacgtatctccccaagtcctctgtggccagctgcatctgtctgaatggtgcgtgaaggctctcagaccttacacaccattttgtaagttatgttttacatgccccgtttttgagactgatctcgatgcaggtggatctccttgagatcctgatagcctgttacaggaatgaagtaaaggtcagttttttttgtattgattttcacagctttgaggaacatgcataagaaatgtagctgaagtagaggggacgtgagagaagggccaggccggcaggccaaccctcctccaatggaaattcccgtgttgcttcaaactgagacagatgggacttaacaggcaatggggtccacttccccctcttcagcatcccccgtaccccactttttgctgaaagaactgccagcaggtaggaccccagaggcccccaaatgaaagcttgaatttcccctactggctctgcgttttgctgagatctgtaggaaaggatgcttcacaaactgaggtagataatgctatgctgtcgttggtatacatcatgaatttttatgtaaattgctctgcaaagcaaattgatatgtttgataaatttatgtttttaggtaaataaaaacttttaaaaagttgtt

SEQ ID NO: 26 - protein, 450 aa, Artificial Sequence
  SYNTHETIC CONSTRUCT
MNLEGGGRGGEFGMSAVSCGNGKLRQWLIDQIDSGKYPGLVWENEEKSIFRIPWKHAGKQDYNREEDAALFKAWALFKGKFREGIDKPDPPTWKTRLRCALNKSNDFEELVERSQLDISDPYKVYRIVPEGAKKGAKQLTLEDPQMSMSHPYTMTTPYPSLPAQVHNYMMPPLDRSWRDYVPDQPHPEIPYQCPMTFGPRGHHWQGPACENGCQVTGTFYACAPPESQAPGVPTEPSIRSAEALAFSDCRLHICLYYREILVKELTTSSPEGCRISHGHTYDASNLDQVLFPYPEDNGQRKNIEKLLSHLERGVVLWMAPDGLYAKRLCQSRIYWDGPLALCNDRPNKLERDQTCKLFDTQQFLSELQAFAHHGRSLPRFQVTLCFGEEFPDPQRQRKLITAHVEPLLARQLYYFAQQNSGHFLRGYDLPEHISNPEDYHRSIRHSSIQE

SEQ ID NO: 27 - DNA, 1353 bp, Artificial Sequence
  SYNTHETIC CONSTRUCT
atgaacctggagggcggcggccgaggcggagagttcggcatgagcgcggtgagctgcggcaacgggaagctccgccagtggctgatcgaccagatcgacagcggcaagtaccccgggctggtgtgggagaacgaggagaagagcatcttccgcatcccctggaagcacgcgggcaagcaggactacaaccgcgaggaggacgccgcgctcttcaaggcttgggcactgtttaaaggaaagttccgagaaggcatcgacaagccggaccctcccacctggaagacgcgcctgcggtgcgctttgaacaagagcaatgactttgaggaactggttgagcggagccagctggacatctcagacccgtacaaagtgtacaggattgttcctgagggagccaaaaaaggagccaagcagctcaccctggaggacccgcagatgtccatgagccacccctacaccatgacaacgccttacccttcgctcccagcccaggttcacaactacatgatgccacccctcgaccgaagctggagggactacgtcccggatcagccacacccggaaatcccgtaccaatgtcccatgacgtttggaccccgcggccaccactggcaaggcccagcttgtgaaaatggttgccaggtgacaggaaccttttatgcttgtgccccacctgagtcccaggctcccggagtccccacagagccaagcataaggtctgccgaagccttggcgttctcagactgccggctgcacatctgcctgtactaccgggaaatcctcgtgaaggagctgaccacgtccagccccgagggctgccggatctcccatggacatacgtatgacgccagcaacctggaccaggtcctgttcccctacccagaggacaatggccagaggaaaaacattgagaagctgctgagccacctggagaggggcgtggtcctctggatggcccccgacgggctctatgcgaaaagactgtgccagagcaggatctactgggacgggcccctggcgctgtgcaacgaccggcccaacaaactggagagagaccagacctgcaagctctttgacacacagcagttcttgtcagagctgcaagcgtttgctcaccacggccgctccctgccaagattccaggtgactctatgctttggagaggagtttccagaccctcagaggcaaagaaagctcatcacagctcacgtagaacctctgctagccagacaactatattattttgctcaacaaaacagtggacatttcctgaggggctacgatttaccagaacacatcagcaatccagaagattaccacagatctatccgccattcctctattcaagaatga

SEQ ID NO: 28 - DNA, 5320 bp, Artificial Sequence
  SYNTHETIC CONSTRUCT
acctcgcactctcagtttcaccgctcgatcttgggacccaccgctgccctcagctccgagtccagggcgagtgcagagcagagcgggcggaggaccccgggcgcgggcgcggacggcacgcgggcatgaacctggagggcggcggccgaggcggagagttcggcatgagcgcggtgagctgcggcaacgggaagctccgccagtggctgatcgaccagatcgacagcggcaagtaccccgggctggtgtgggagaacgaggagaagagcatcttccgcatcccctggaagcacgcgggcaagcaggactacaaccgcgaggaggacgccgcgctcttcaaggcttgggcactgtttaaaggaaagttccgagaaggcatcgacaagccggaccctcccacctggaagacgcgcctgcggtgcgctttgaacaagagcaatgactttgaggaactggttgagcggagccagctggacatctcagacccgtacaaagtgtacaggattgttcctgagggagccaaaaaaggagccaagcagctcaccctggaggacccgcagatgtccatgagccacccctacaccatgacaacgccttacccttcgctcccagcccaggttcacaactacatgatgccacccctcgaccgaagctggagggactacgtcccggatcagccacacccggaaatcccgtaccaatgtcccatgacgtttggaccccgcggccaccactggcaaggcccagcttgtgaaaatggttgccaggtgacaggaaccttttatgcttgtgccccacctgagtcccaggctcccggagtccccacagagccaagcataaggtctgccgaagccttggcgttctcagactgccggctgcacatctgcctgtactaccgggaaatcctcgtgaaggagctgaccacgtccagccccgagggctgccggatctcccatggacatacgtatgacgccagcaacctggaccaggtcctgttcccctacccagaggacaatggccagaggaaaaacattgagaagctgctgagccacctggagaggggcgtggtcctctggatggcccccgacgggctctatgcgaaaagactgtgccagagcaggatctactgggacgggcccctggcgctgtgcaacgaccggcccaacaaactggagagagaccagacctgcaagctctttgacacacagcagttcttgtcagagctgcaagcgtttgctcaccacggccgctccctgccaagattccaggtgactctatgctttggagaggagtttccagaccctcagaggcaaagaaagctcatcacagctcacgtagaacctctgctagccagacaactatattattttgctcaacaaaacagtggacatttcctgaggggctacgatttaccagaacacatcagcaatccagaagattaccacagatctatccgccattcctctattcaagaatgaaaaatgtcaagatgagtggttttctttttccttttttttttttttttttgatacggggatacggggtcttgctctgtctcccaggctggagtgcagtgacacaatctcagctcactgtgacctccgcctcctgggttcaagagactctcctgcctcagcctccctggtagctgggattacaggtgtgagccactgcacccacccaagacaagtgattttcattgtaaatatttgactttagtgaaagcgtccaattgactgccctcttactgttttgaggaactcagaagtggagatttcagttcagcggttgaggagaattgcggcgagacaagcatggaaaatcagtgacatctgattggcagatgagcttatttcaaaaggaagggtggctttgcatttcttgtgttctatagactgccatcattgatgatcactgtgaaaattgaccaagtgatgtgtttacatttactgaaatgtgctctttaatttgttgtagattaggtcttgctggaagacagagaaaacttgcctttcagtattgacactgactagagtgatgactgcttgtaggtatgtctgtgccatttctcagggaagtaagatgtaaattgaagaagcctcacacgtaaaagaaatgtattaatgtatgtaggagctgcagttcttgtggaagacacttgctgagtgaaggaaatgaatctttgactgaagccgtgcctgtagccttggggaggcccatcccccacctgccagcggtttcctggtgtgggtccctctgccccaccctccttcccattggctttctctccttggcctttcctggaagccagttagtaaacttcctattttcttgagtcaaaaaacatgagcgctactcttggatgggacatttttgtctgtcctacaatctagtaatgtctaagtaatggttaagttttcttgtttctgcatctttttgaccctcattctttagagatgctaaaattcttcgcataaagaagaagaaattaaggaacataaatcttaatacttgaactgttgcccttctgtccaagtacttaactatctgttcccttcctctgtgccacgctcctctgtttgcttggctgtccagcgatcagccatggcgacactaaaggaggaggagccggggactcccaggctggagagcactgccaggacccaccactggaagcaggatggagctgactacggaactgcacactcagtgggctgtttctgcttatttcatctgttctatgcttcctcgtgccaattatagtttgacagggccttaaaattacttggctttttccaaatgcttctatttatagaatcccaaagacctccacttgcttaagtatacctatcacttacatttttgtggttttgagaaagtacagcagtagactggggcgtcacctccaggccgtttctcatactacaggatatttactattactcccaggatcagcagaagattgcgtagctctcaaatgtgtgttcctgcttttctaatggatattttaaattcattcaacaagcacctagtaagtgcctgctgtatccctacattacacagttcagcctttatcaagcttagtgagcagtgagcactgaaacattattttttaatgtttaaaaagtttctaatattaaagtcagaatattaatacaattaatattaatattaactacagaaaagacaaacagtagagaacagcaaaaaaataaaaaggatctccttttttcccagcccaaattctcctctctaaaagtgtccacaagaaggggtgtttattcttccaacacatttcacttttctgtaaatatacataaacttaaaaagaaaacctcatggagtcatcttgcacacactttcatgcagtgctctttgtagctaacagtgaagatttacctcgttctgctcagaggccttgctgtggagctccactgccatgtacccagtagggtttgacatttcattagccatgcaacatggatatgtattgggcagcagactgtgtttcgtgaactgcagtgatgtatacatcttatagatgcaaagtattttggggtatattatcctaagggaagataaagatgatattaagaactgctgtttcacggggcccttacctgtgaccctctttgctgaagaatatttaaccccacacagcacttcaaagaagctgtcttggaagtctgtctcaggagcaccctgtcttcttaattctccaagcggatgctccatttcaattgctttgtgacttcttcttctttgtttttttaaatattatgctgctttaacagtggagctgaattttctggaaaatgcttcttggctggggccactacctcctttcctatctttacatctatgtgtatgttgactttttaaaattctgagtgatccagggtatgacctagggaatgaactagctatgaaatactcagggttaggaatcctagcacttgtctcaggactctgaaaaggaacggcttcctcattccttgtcttgataaagtggaattggcaaactagaatttagtttgtactcagtggacagtgctgttgaagatttgaggacttgttaaagagcactgggtcatatggaaaaaatgtatgtgtctcccaggtgcatttcttggtttatgtcttgttcttgagattttgtatatttaggaaaacctcaagcagtaattaatatctcctggaacactatagagaaccaagtgaccgactcatttacaactgaaacctaggaagcccctgagtcctgagcgaaaacaggagagttagtcgccctacaggaaacccagctagactattgggtatgaactaaaaagagactgtgccatggtgagaaaaatgtaaaatcctacagtggaatgagcagcccttacagtgttgttaccaccaagggcaggtaggtattagtgtttgaaaaagctggtctttgagcgagggcataaatacagctagccccaggggtggaacaactgtgggagtcttgggtactcgcacctcttggctttgttgatgctccgccaggaaggccacttgtgtgtgcgtgtcagttacttttttagtaacaattcagatccagtgtaaacttccgttcattgctctccagtcacatgcccccacttccccacaggtgaaagtttttctgaaagtgttgggattggttaaggtctttatttgtattacgtatctccccaagtcctctgtggccagctgcatctgtctgaatggtgcgtgaaggctctcagaccttacacaccattttgtaagttatgttttacatgccccgtttttgagactgatctcgatgcaggtggatctccttgagatcctgatagcctgttacaggaatgaagtaaaggtcagttttttttgtattgattttcacagctttgaggaacatgcataagaaatgtagctgaagtagaggggacgtgagagaagggccaggccggcaggccaaccctcctccaatggaaattcccgtgttgcttcaaactgagacagatgggacttaacaggcaatggggtccacttccccctcttcagcatcccccgtaccccactttttgctgaaagaactgccagcaggtaggaccccagaggcccccaaatgaaagcttgaatttcccctactggctctgcgttttgctgagatctgtaggaaaggatgcttcacaaactgaggtagataatgctatgctgtcgttggtatacatcatgaatttttatgtaaattgctctgcaaagcaaattgatatgtttgataaatttatgtttttaggtaaataaaaacttttaaaaagttgtt

SEQ ID NO: 29 - RNA, 19 nt, Artificial Sequence
  SYNTHETIC CONSTRUCT
ccacagaucuauccgccau

SEQ ID NO: 30 - RNA, 19 nt, Artificial Sequence
  SYNTHETIC CONSTRUCT
ugucagagcugcaagcguu

SEQ ID NO: 31 - RNA, 19 nt, Artificial Sequence
  SYNTHETIC CONSTRUCT
gaaaaugguugccagguga

(19) United States
(12) Patent Application Publication
(10) Pub. No.: US 2021/0380683 A1
(43) Pub. Date: Dec. 9, 2021

(54) MODULATION OF IRF-4 AND USES THEREOF

(71) Applicant: THE METHODIST HOSPITAL SYSTEM, Houston, TX (US)

(72) Inventors: Wenhao CHEN, Houston, TX (US); Xian Chang LI, Houston, TX (US)

(21) Appl. No.: 17/046,843

(22) PCT Filed: Apr. 12, 2019
(86) PCT No.: PCT/US19/27162

§ 371 Date: Oct. 12, 2020

Related U.S. Application Data
(60) Provisional application No. 62/656,576, filed on Apr. 12, 2018.

(51) Int. Cl. C07K 16/28; A61K 35/17; A61P 37/06

(52) U.S. Cl. CPC C07K 16/2803; A61K 2039/505; A61P 37/06; A61K 35/17

(57) ABSTRACT

Disclosed are methods and compositions for inhibiting IRF4 in T-cells, thereby improving transplant outcomes or treating an autoimmune disease such as a myelination disorder. Thus, disclosed are methods to improve a transplant outcome in a recipient of a transplant comprising inhibiting IRF4 in T-cells of the recipient, thereby improving the transplant outcome. In some embodiments, IRF4 can be inhibited by administering an IRF4 inhibitor (e.g., trametinib or anti-IRF4 siRNA), and/or by adoptive transfer of T-cells having IRF4 inhibition. In some embodiments, the T-cells are CD4+ Tcells, and the infiltration thereof into a transplant can be reduced. Also disclosed are methods of treating a subject with a myelination disorder (e.g., multiple sclerosis or encephalomyelitis) comprising inhibiting IRF4.

### CROSS REFERENCE TO RELATED APPLICATIONS

This application claims the benefit of U.S. Provisional Patent Application Ser. No. 62/656,576 filed Apr. 12, 2018, which is expressly incorporated herein by reference.

### STATEMENT REGARDING FEDERALLY SPONSORED RESEARCH

This invention was made with government support under Grant No. R01AI106200 awarded by the National Institutes of Health. The Government has certain rights in the invention.

### FIELD

The disclosure generally relates to improving transplant and graft outcomes in a recipient, and to treating autoimmune diseases, by modulating alloimmunity and autoimmunity responses.

### BACKGROUND

In organ transplantation, CD4+ but not CD8+ T cells are essential for allorejection, as it has been shown that CD4+ T cells are necessary and sufficient for mediating acute rejection of heart and kidney allografts (Bolton et al., 1989; Krieger et al. 1996). Among CD4+ T cell subsets, alloreactive T helper 1 (Th-1) cells have been shown to cause allograft damage directly through Fas-Fas ligand-mediated cytotoxicity, or indirectly through inducing delayed type hypersensitivity by macrophages and promoting the activity of cytotoxic CD8+ T cells (Liu et al. 2013). Th17 cells also mediate allograft rejection, which has been demonstrated in recipient mice lacking T-bet, the master regulator of Th1 cell differentiation (Yuan et al., 2008). T follicular helper (Tfh) cells contribute to allograft rejection by promoting alloantibody responses (Conlon et al., 2012). By contrast, CD4+Foxp3+ regulatory (Treg) cells protect the transplanted organs from rejection in many experimental models (Miyahara et al., 2012; Safinia et al., 2015).

T cell dysfunction, such as exhaustion and anergy, represents distinct T cell differentiation states following antigen encounter (Schietinger and Greenberg, 2014). The dysfunctional differentiation of T cells involves the transcriptional induction of essential negative regulators that inhibit T cell function (Fathman and Lineberry, 2007; Wherry and Kurachi, 2015). For instance, dysfunctional T cells that arise during certain chronic infections and cancers sustainably express various inhibitory receptors, including programmed cell death protein 1 (PD-1), CD160, lymphocyte-activation gene 3 (LAG3), B and T lymphocyte attenuator (BTLA), and cytotoxic T lymphocyte antigen 4 (CTLA-4) (Crawford et al., 2014; Schietinger et al., 2016). These receptors exert inhibitory effects on T cell function; blockade of PD-1, programmed death-ligand 1 (PD-L1), or CTLA-4 has been successfully used to treat several cancer types by reversing T cell dysfunction (Zarour, 2016). Transcription factors T-bet, Blimp-1, NFAT, and FOXO1 regulate PD-1 expression and have been implicated in T cell exhaustion and dysfunction (Wherry and Kurachi, 2015).

Interferon regulatory factor 4 (IRF4) is a member of the IRF family of transcription factors and is preferentially expressed in hematopoietic cells. It plays essential roles in many aspects of T cell, B cell and dendritic cell differentiation and function (Huber and Lohoff, 2014; Ochiai et al., 2013; Vander Lugt et al., 2014). In T cells, IRF4 is promptly expressed within hours following TCR stimulation, and its expression level is TCR affinity dependent (Man et al., 2013). IRF4 controls the differentiation of Th2, Th9, Th17, Tfh, Treg, and cytotoxic effector CD8+ T cells (Bollig et al., 2012; Brustle et al., 2007; Cretney et al., 2011; Huber et al., 2008; Staudt et al., 2010; Yao et al., 2013; Zheng et al., 2009). Irf4-deficient T cells exhibit a functional defect in T cell-mediated responses, including microbial infection, allergy, graft-versus-host reaction, and autoimmunity (Brustle et al., 2007; Grusdat et al., 2014; Huber and Lohoff, 2014; Mittrucker et al., 1997; Staudt et al., 2010).

### SUMMARY

The disclosed subject matter relates to manipulating levels of IRF4 to improve transplant outcomes and for treating autoimmune diseases. More specifically, the disclosure relates to inhibiting IRF4 in T-cells, thereby improving transplant outcomes or treating an autoimmune disease.

Current donation techniques for organs, tissues, blood, cells, and other biologics require careful selection of very specific donors having genetic or phenotypic criteria compatible with the recipient. For example, organ and tissue transplants require specific selection of a donor having a major histocompatibility complex (MHC) profile which is identical (as in the case of genetically identical twins) or very similar to the MHC profile of the recipient. As dissimilarity increases between the MHC profiles of the donor and the recipient, so does the risk for the recipient's immune system to reject the transplant.

Certain human leukocyte antigens (HLA) are proteins located on the surface of leukocytes (sometimes called white blood cells) and make up a part of the MHC in humans. HLA proteins are primary regulators of the human immune response, and it is in part the genes encoding these proteins which are screened for compatibility between donors and recipients. However, HLA proteins are highly polymorphic: there are at least 59 different HLA-A proteins, 118 different HLA-B proteins, and 124 different HLA-DR proteins. This high variability makes it difficult to identify donors having very similar MHC profiles as that of the recipient.

Prior methods to ameliorate transplant rejection by the recipient's immune system (referred to as alloimmunity) include use of immunosuppressant therapeutics such as glucocorticoids, methotrexate, azathioprine, dactinomycin, and anti-CD3 and anti-IL2 antibodies, among others. However, current methods to modulate alloimmune responses remain limited to use in transplants in which the recipient has the same or similar MHC profile as that of the donor, severely limiting the pool of donors for any given recipient. Further, use of immunosuppressant therapeutics increases the recipient's risk of secondary infection because the recipient's immune system is suppressed from forming effective responses to invading pathogens.

Autoimmunity, wherein an individual's immune system attacks an individual's own biologics (e.g., cells or tissues), presents similar conceptual issues as alloimmunity. Although autoimmunity does not include a mismatched MHC profile, effector immune cells nonetheless recognize one or more of the individual's antigens as being foreign and mount a response thereto. As such, immunosuppressant therapeutics are typically used to treat autoimmune diseases as well, presenting similar risks of secondary infection and other complications.

The compositions and methods disclosed herein address these and other needs in part by modulating alloimmune and autoimmune responses. By inhibiting IRF4 in T-cells, the T-cell mediated responses to a transplant, graft, or autoimmunity-triggering antigen can be decreased, thereby improving outcomes in transplant recipients and individuals suffering from autoimmunity disorders. For example, transplant recipients can have decreased transplant rejection and increased survival, even when transplanted with tissues or organs from a MHC mismatched donor. As another example, certain autoimmunity patients such as those suffering from myelination disorders can have improved motor function and/or reduced paralysis.

In one aspect, disclosed herein are methods to improve a transplant outcome in a recipient of a transplant comprising inhibiting IRF4 in T-cells of the recipient, thereby improving the transplant outcome. In some embodiments, IRF4 is inhibited by administering to the recipient an IRF4 inhibitor. In some embodiments, the IRF4 inhibitor comprises a MEK 1/2 inhibitor, which can be trametinib. In some embodiments, the IRF4 inhibitor comprises an anti-IRF4 siRNA. In some embodiments, IRF4 polypeptide expression is inhibited by at least 50% compared to a control, which can be an unmodified T-cell of the recipient. In some embodiments, the T-cells comprise activated T-cells, and/or CD4+ T-cells. In some embodiments, the recipient comprises a MHC profile which is fully mismatched compared to a donor of the transplant. In some embodiments, the improved transplant outcome can be reduced inflammation or reduced T-cell infiltration in the transplant, or acceptance of the transplant by the recipient for at least 100 days. In some embodiments, IRF4 is inhibited prior to transplantation.

Also disclosed herein are methods of treating a subject with a myelination disorder comprising inhibiting IRF4. In some embodiments, the myelination disorder comprises multiple sclerosis or encephalomyelitis.

Also disclosed are methods of increasing T-cell dysfunction in a subject comprising inhibiting IRF4, wherein the subject has an autoimmune disease or is a recipient of a transplant.

Also disclosed are methods to identify a compound which inhibits IRF4 comprising contacting one or more T-cells with the compound; and measuring IRF4 expression in the one or more T-cells; wherein reduced IRF4 expression compared to a control indicates the compound inhibits IRF4; and wherein the control comprises one or more T-cells which are not contacted with the compound.

Also disclosed are methods to measure IRF4 expression in T-cells of a subject prescribed to receive a transplant comprising obtaining T-cells from the subject; and measuring IRF4 expression in the T-cells.

Additional aspects and advantages of the disclosure will be set forth, in part, in the detailed description and any claims which follow, and in part will be derived from the detailed description or can be learned by practice of the various aspects of the disclosure. The advantages described below will be realized and attained by means of the elements and combinations particularly pointed out in the appended claims. It is to be understood that both the foregoing general description and the following detailed description are exemplary and explanatory only and are not restrictive of the disclosure.

### BRIEF DESCRIPTION OF THE DRAWINGS

The accompanying drawings, which are incorporated in and constitute a part of this specification, illustrate certain examples of the present disclosure and together with the description, serve to explain, without limitation, the principles of the disclosure. Like numbers represent the same element(s) throughout the figures.

FIG. 1A through 1D show IRF4 is overexpressed in graft-infiltrating T cells, and Irf4-deficient T cells do not mediate heart allograft rejection. Flow cytometry analysis of IRF4 expression in CD4+(FIG. 1A; left panel) and CD8+(FIG. 1A; right panel) T cells from the spleens and allografts of WT B6 recipient mice at 7 days after Balb/c heart transplantation (HTx). CD4+ and CD8+ T cells from the spleens of naïve B6 mice were used as controls. Representative histograms (FIG. 1A) and IRF4 mean fluorescence intensity (MFI) values (FIG. 1B) were shown. Data in (FIG. 1B) are presented as mean±SD (n=4). **P<0.01; unpaired Student's t-test. Data are representative of two independent experiments. WT B6 and Irf4^{fl/fl}Cd4-Cre mice were transplanted with Balb/c hearts. FIG. 1C shows the percentage of allograft survival after transplantation (n=6). **P<0.01; Mann-Whitney test. FIG. 1D shows H&E stained sections of heart allografts harvested from WT B6 recipients at day 7 post-transplant, or from Irf4^{fl/fl}Cd4-Cre recipients at day 7 or day 100 post-transplant. Three mice per group were analyzed, with ten graft sections per mouse. Two representative images (top and bottom) per group are shown.

FIGS. 2A through 2D show the association of T-cell activation markers with transplant rejection and effects of IRF4 deficiency on phenotypic changes of T cells in transplanted mice. Balb/c hearts were transplanted into B6 recipients (HTx group). At day 7 post-transplant, CD4+ and CD8+T cells from the spleens and grafts of recipient mice were analyzed by flow cytometry. CD4+ and CD8+ T cells from the spleens of un-transplanted B6 mice (Naïve group) were used as controls. FIG. 2A shows expression of CD62L and CD44 on CD4+(top) or CD8+(bottom) T cells. FIG. 2B shows expression of CD98 and intracellular (I) GLUT1 by CD4+ or CD8+ T cells. Data are representative of three independent experiments. In FIGS. 2C and 2D, WT B6 and Irf4^{fl/fl} Cd4-Cre mice were transplanted with Balb/c heart allografts (HTx groups) or left un-transplanted (no Tx groups). FIG. 2C shows splenocytes were isolated from the indicated groups and analyzed by flow cytometry at day 7 post-transplant. In the top row of contour plots, % CD4+ and CD8+ cells among CD45+ cells are shown. Other contour plots indicate the expression of CD62L and CD44 on CD4+ or CD8+ T cells, or co-expression of Foxp3 and CD25 by CD4+ T cells. Bar graphs present the numbers of CD4+, CD8+, CD4+CD62L^{low} CD44+, CD4+ Foxp3+, CD8+CD62L^{low} CD44+ cells in the spleens. FIG. 2D shows splenocytes were analyzed by flow cytometry at day 9 post-transplant. Frequencies and numbers of CD4+ CXCR5+Bc16+T_{FH} cells, CD45+CD19^{low} CD138+ plasma cells, and CD19+GL7+PNA+GC B cells are shown. **P<0.01 (unpaired student's t-test). Data are mean±SD and are representative of two experiments with three to four mice in each.

FIGS. 3A through 3H show the dysfunction of Irf4-deficent T cells in transplantation is correlated with impaired cytokine production and graft infiltration. FIG. 3A shows Balb/c heart allograft survival in Irf4^{fl/fl}Cd4-Cre mice that were adoptively transferred with 2 million (M) or 20 M indicated T cells. FIG. 3B shows Balb/c heart allograft survival in Irf4^{fl/fl}Cd4-Cre mice that were treated with rat IgG or an anti-CD25 (αCD25) mAh on indicated days. FIG. 3C is a schematic of the experimental design in which Rag1^{-/-} mice were co-injected with 2×10⁷ (20M) CD45.1+WT and 20M CD45.2+(CD45.1−) Irf4^{-/-} T cells on day −1, and received Balb/c heart allografts on day 0. Splenocytes and graft-infiltrating cells were isolated on day 9 for flow cytometry analysis. FIG. 3D shows representative contour plots showing percent transferred CD45.1+WT and CD4^{-/-} T cells (CD4+ or CD4−, gated on CD3+ cells) in spleens on day −1 (FIG. 3D; left panel) and day 9 (FIG. 3D; right panel). The left panel of FIG. 3E shows percent CD4+(CD3+CD8−) and CD8+(CD3+CD8+) T cells among CD45+ graft-infiltrating cells on day 9, and the right plots display percent CD45.1+WT and CD45.1− Irf4^{-/-} cell populations among those CD4+(FIG. 3E, upper right panel) and CD8+(FIG. 3E, lower right panel) infiltrating T cells. FIG. 3F shows the expression of the markers (from left to right) CD62L, CD44, CD25, and KLRG1 in CD4+(top row) and CD8+(bottom row) populations of transferred CD45.1+WT and CD45.1− Irf4^{-/-} T cells in spleens on day 9. FIG. 3G depicts the percent expression of the indicated molecules in the contour plots (from left to right) IFNγ, IL-4, IL-17A, and Foxp3 in co-transferred CD45.1+WT and CD45.1− Irf4^{-/-} CD4+ T cells in spleens on day 9, and depicts quantification of the results in bar graph form (FIG. 3G, bottom panel). FIG. 3H shows the percent expression of the indicated molecules in the contour plots (from left to right) IFNγ, IL-17A, Perforin, and Granzyme B in co-transferred CD45.1+WT and CD45.1− Irf4^{-/-} CD4+ T cells in spleens on day 9, and depicts quantification of the results in bar graph form (FIG. 3H, bottom panel). For FIGS. 3G and 3H, data are mean±SD. **P<0.01; unpaired student's t-test. For FIGS. 3C-3H, data are representative of three independent experiments with three to four mice in each group.

FIGS. 4A through 4F show the dysfunction of Irf4-deficent T cells in transplantation is correlated with impaired cytokine production and graft infiltration. Rag1^{-/-} mice were injected with 5 million CD4+ or CD8+ T cells sorted from either WT or Irf4^{-/-} mice on day −1 and received Balb/c heart allografts on day 0. Splenocytes and graft-infiltrating cells were isolated on day 9 for flow cytometry analysis. FIG. 4A shows a schematic of the experimental design. FIG. 4B shows percent CD4+ and percent CD8+ T cells among CD45+ graft-infiltrating cells in recipients injected with indicated CD4+(left two panels) or CD8+(right two panels) T cells. FIGS. 4C and 4D show expression of indicated markers on transferred CD4+(FIG. 4C) or CD8+(FIG. 4D) cell populations in spleens. FIGS. 4E and 4F show plots (top) and the bar graph (bottom) depicting the percentage expression of the indicated molecules in transferred CD4+(FIG. 4E) or CD8+(FIG. 4F) T cells in spleens. Data are mean+/−SD (FIGS. 4E and 4F) and are representative of two independent experiments. *P<0.05; **P<0001; unpaired student's t-test.

FIGS. 5A through 5E show IRF4 represses a set of molecules associated with CD4+ T cell dysfunction. FIG. 5A shows flow cytometry analysis of the indicated cell surface molecules expressed on naïve WT CD4+ T cells (gray shades), or on activated WT (black lines) or Irf4^{-/-} (red lines) CD4+ T cells one day after stimulation with B6 APCs (mitomycin C-treated, T-cell-depleted B6 splenocytes) and soluble anti-CD3 mAh. Mean Fluorescence Intensities (MFI) for each of the indicated cell surface molecules in the above referenced cell types are quantified in the bar graphs of FIG. 5A. In additional experiments (FIGS. 5B-5E), WT and Irf4^{-/-} CD4+ T cells were activated for 2 days, RNA was analyzed by microarray and quantitative real-time PCR, and Helios expression was analyzed by flow cytometry. FIG. 5B depicts a heat map showing the normalized expression scores (relative to row mean) of selected genes from WT or Irf4^{-/-} CD4+ T cells. Two RNA samples of each group were obtained from two independent culture experiments of pooled T cells from n=3 mice per sample. FIG. 5C shows Gene Ontology (GO) categories enrichment analysis of 438 upregulated genes in Irf4^{-/-} CD4+ T cells in accordance with biological process. The horizontal axis shows −log₁₀ of the P-value. FIG. 5D shows the relative changes of mRNA expression of the indicated genes in Irf4^{-/-} CD4+ T cells compared to WT CD4+ T cells, as determined by quantitative real-time PCR. Data are mean±SD. FIG. 5E shows flow cytometry analysis of Helios expression in WT (left contour plot) and Irf4^{-/-} (right contour plot) CD4+ T cells, which is quantified in the accompanying bar graph. Data in FIGS. 5A, 5D, and 5E are representative of three experiments with triplicate samples.

FIGS. 6A through 6H show upregulation of PD-1 in activated Irf4^{-/-} CD4+ T cells through increased chromatin accessibility and Helios binding at PD-1 cis-regulatory elements. FIG. 6A depicts histograms showing PD-1 expression (shades and lines) and MFI (numbers) on freshly isolated naïve WT CD4+ T cells (gray), or on activated WT (black) or Irf4^{-/-} (red) CD4+ T cells on the indicated days after stimulation with B6 APCs and soluble anti-CD3 mAh. The line graph of FIG. 6A displays change in PD-1 MFI with time after activation. FIG. 6B shows PD-1 expression on co-cultured CD45.1+WT and CD45.1− Irf4^{-/-} CD4+ T cells 3 days after activation. FIG. 6C shows PD-1 expression on activated Irf4^{-/-} CD4+ T cells transduced with a retroviral vector expressing GFP alone (Ctrl) or with retrovirus expressing Irf4-GFP (IRF4). Numbers in contour plots (left) and the bar graph (right) indicate PD-1 MFI of gated GFP+ cells. The bar graph to the right in FIG. 6C depicts quantified results when gating on GFP+ cells. FIG. 6D shows a schematic of the genomic arrangement near the Pdcd1 chromosomal locus, and ChIP analysis of H3Ac (upper left panel), H4Ac (upper right panel), H3K4me3 (lower left panel), and H3K9me3 (lower right panel) at the PD-1 cis-regulatory elements (−3.7, CR-C, CR-B, and +17.1) in WT (empty bars) and Irf4^{-/-} (solid bars) CD4+ T cells 2 days after activation. FIG. 6E shows PD-1 and Helios expressions on WT (left panel) and Irf4^{-/-} (right panel) CD4+ T cells at 2 days after activation. FIG. 6F shows ChIP analysis of the enrichment of Helios at the PD-1 cis-regulatory elements in WT (empty bars) and Irf4^{-/-} (solid bars) CD4+ T cells at 2 days after activation. FIG. 6G shows PD-1 expression on activated WT CD4+ T cells transduced with a retroviral vector expressing GFP alone (Ctrl; left panel) or with retrovirus expressing Ikzf2-GFP (Helios; right panel). Numbers in contour plots (top) and the bar graph (bottom) indicate PD-1 MFI of gated GFP+ cells. FIG. 6H shows Helios (top three panels) and PD-1 (bottom two panels) expression by GFP+ Irf4^{-/-} CD4+ T cells that were transduced with a retroviral vector co-expressing GFP and shRNA sequences for Helios (sh-Helios; top right panel) or containing GFP alone (sh-Ctrl; top left panel). Numbers in contour plots and the bar graphs indicate Helios and PD-1 MFI of gated GFP+ cells. *P<0.05 and **P<0.01 (unpaired student's t-test). Data are representative of three independent experiments.

FIGS. 7A through 7F show ChIP assays for binding of IRF4 to putative binding sites upstream of pdcd1, a site in the Ikzf2 intron, and the PD-1 regulatory regions. Shown are putative IRF4 binding sites (FIG. 7A, marked in light grey; SEQ ID NOG shows the 2 kb promoter sequence upstream of pdcd1 in mouse) upstream of pdcd1, and their related primer sequences (FIG. 7B) used for real-time PCR analysis in ChIP assays. SEQ ID NON and SEQ ID NOG; SEQ ID NOG and SEQ ID NO:7; SEQ ID NOG and SEQ ID NO:9; SEQ ID NO: 10 and SEQ ID NO: 11; SEQ ID NO: 12 and SEQ ID NO: 13; disclose primer pairs used for real time PCR analysis in ChIP assays for CHIP5, CHIP4, CHIP3, CHIP2, and CHIP1, respectively. FIG. 7C and SEQ ID NO: 14 show IRF4 binding site in Ikzf2 intron, and its related primers used for real-time PCR in ChIP assays. FIG. 7D shows ChIP assays for binding of IRF4 to putative sites upstream of pdcd1 and a site in the Ikzf2 intron in WT and Irf4^{-/-} CD4+ T cells 2 days after activation. ChIP assay (FIG. 7E) for binding of IRF4 to −3.7, CR-C, CR-B, and +17.1 PD-1 regulatory regions in WT and Irf4^{-/-} CD4+ T cells 2 days after activation is shown, as are primer sets (FIG. 7F) used for real-time PCR in the ChIP assay of FIG. 7E. SEQ ID NO: 15 and SEQ ID NO: 16; SEQ ID NO: 17 and SEQ ID NO: 18; SEQ ID NO: 19 and SEQ ID NO:20; SEQ ID NO:21 and SEQ ID NO:22; SEQ ID NO:23 and SEQ ID NO:24; disclose primer pairs used for real time PCR analysis in ChIP assays of FIG. 7E for control, −3.7, CR-C, CR-B, and +17.1 PD-1 regulatory regions, respectively. **P<0.01 (unpaired student's t-test). Data in FIG. 7D and FIG. 7E are mean±SD and are representative of three experiments.

FIGS. 8A through 8J show responsiveness to checkpoint blockade defines the dysfunctional states of Irf4-deficient T cells after transplantation. FIG. 8A shows Balb/c heart graft survival in Irf4^{fl/fl}Cd4-Cre mice that were adoptively transferred with 2×10⁷ WT or Irf4^{-/-} TEa cells on day −1. FIG. 8B shows a schematic of the experimental design in which CD45.1+ congenic mice were transferred with 5×10⁶ (5M) CellTrace Violet (CTV)-labeled CD45.2+WT or Irf4^{-/-} TEa cells on day −1, received Balb/c heart transplants (HTx) or left un-transplanted (no Tx) on day 0, followed by analysis of splenocytes on day 6. Contour plots in FIG. 8B show co-expression of CD45.2 with CTV (top row) or PD-1 (middle row), gated on CD4+ cells; or show co-expression of Foxp3 with intracellular CTLA-4, gated on CD4+CD45.2+ TCRα2+ TEa cells (bottom row). Histograms display CTV (top), PD-1 (middle), and intracellular CTLA-4 (bottom) expressions, gated on TEa cells. Data are representative of three experiments. FIG. 8C shows Balb/c heart graft survival in Irf4^{fl/fl}Cd4-Cre mice that were treated with rat IgG, anti-PD-L1, anti-CTLA-4, or anti-PD-L1 plus anti-CTLA-4 mAbs on days 0, 3, and 5 post-transplant. FIG. 8D shows dilution of CTV, which indicates proliferation of CD45.1+CD4+ T cells in the absence or presence of Treg cells from the indicated groups. FIG. 8E shows quantification of the results of FIG. 8D presented as division index. In another experiment, Irf4^{fl/fl}Cd4-Cre mice were transplanted with Balb/c hearts and treated with anti-PD-L1 plus anti-CTLA-4 mAbs, together with 400 μg anti-CD4 or anti-CD8 depleting mAh on days 0, 3, and 5. The counter plots of FIG. 8F show efficacy of Control (left panel) or CD4+(middle panel) or CD8+(right panel) T cell depletion on day 4. FIG. 8G shows heart graft survival in the experiment of FIG. 8F. FIG. 8H shows Balb/c heart graft survival in Irf4^{fl/fl}Cd4-Cre mice that were treated with anti-PD-L1 plus anti-CTLA-4 mAbs starting from day 0 (on days 0, 3, and 5), day 7 (on days 7, 10, and 12), or day 30 (on days 30, 33, and 35) post-transplant. The histogram of FIG. 8I shows percent GFP+ cells in CD4+CD69+ Irf4^{-/-} cells following 3-day stimulation with Balb/c splenic dendritic cells (DCs) and 1-day IRF4-GFP viral transduction. FIG. 8J shows Balb/c heart graft survival in Irf4^{fl/fl}Cd4-Cre mice that were transferred on day 1 with 1×10⁶ GFP+ Irf4^{-/-} CD4 T cells (transduced with IRF4-GFP or GFP-Ctrl). P<0.05 and **P<0.01; Mann-Whitney test (FIGS. 8A, 8C, 8G, 8H and 8J).

FIGS. 9A and 9B show effects of checkpoint blockade on Irf4-deficient CD4+ T cells in heart-transplanted mice. Irf4^{fl/fl}Cd4-Cre recipients were transplanted with Balb/c hearts (HTx groups) and treated with anti-PD-L1 plus anti-CTLA-4 mAbs (αPD-L1+αCTLA-4) or rat IgG on days 0, 3, and 5 post-transplant. Un-transplanted WT (no Tx group) mice were used as controls. FIG. 9A depicts contour plots showing the expressions of indicated molecules by CD4+ splenocytes from un-transplanted WT B6 mice or transplanted Irf4^{fl/fl}Cd4-Cre recipients at 7 days post-transplant. FIG. 9B depicts bar graphs showing the frequencies or MFIs of CD4+ splenocytes expressing the indicated molecules. *P<0.05, **P<0.01 (unpaired student's t-test).

FIGS. 10A through 10C show checkpoint blockade reverses the initial dysfunction of Irf4-deficient CD4+ T cells by restoring their ability to undergo proliferation and secrete IFN-γ. FIG. 10A is a schematic showing the experimental design wherein CD45.1+ congenic mice were transferred with 5×10⁶ (5M) CD45.2+WT or Irf4^{-/-} TEa cells on day −1, received Balb/c heart transplants (HTx) or left un-transplanted (no Tx) on day 0, treated with rat IgG (Irf4^{-/-} TEa (IgG)) or anti-PD-L1 plus anti-CTLA-4 mAbs (Irf4^{-/-} TEa (P+C)) on days 0, 3, and 5, followed by flow cytometry analysis of TEa cells in spleens on day 6. FIG. 10B shows transferred TEa cells among CD4+ splenocytes (top row) and expression of the indicated molecules by TEa splenocytes (all other rows) on day 6 post-transplant. FIG. 10C shows percent TEa cells among CD4+ splenocytes and numbers of TEa splenocytes (top row), and percent Ki67+, CD98 MFI, GLUT1 MFI, CD71 MFI, percent IFN-γ+, and percent Foxp3+ of TEa cells (other rows as indicated). *P<0.05 (unpaired student's t-test). Data are mean±SD (FIG. 10C) and are representative of three experiments (FIGS. 10B and 10C).

FIGS. 11A and 11 B show effects of checkpoint blockade on Irf4^{-/-} TEa cells in transplanted mice. FIG. 11A shows the gating strategy for flow cytometry analysis of transferred CD45.2+ TEa cells in CD45.1+ congenic mice. As shown in FIG. 10A, CD45.1+ mice were transferred with 5×10⁶ (5M) CD45.2+WT or Irf4^{-/-} TEa cells on day −1, and transplanted with Balb/c hearts (HTx) or left un-transplanted (no Tx) on day 0. Recipient mice transferred with Irf4^{-/-} TEa cells were further treated with rat IgG (Irf4^{-/-} TEa (IgG)) or anti-PD-L1 plus anti-CTLA-4 mAbs (Irf4^{-/-} TEa (P+C)) on days 0, 3, and 5, followed by flow cytometry analysis on day 6. FIG. 11B depicts contour plots showing expressions of indicated molecules by transferred CD4+TCRα2+CD45.2+ TEa cells in spleens. Bar graphs show the frequencies of TEa cells expressing the indicated molecules. *P<0.05, **P<0.01 (unpaired student's t-test). Data are mean±SD and are representative of three experiments.

FIGS. 12A through 12G show trametinib inhibits IRF4 expression in T cells, prevents experimental autoimmune encephalomyelitis (EAE) development, and prolongs allograft survival. FIG. 12A shows IRF4 expression (left panel) and MFI (right panel) in freshly isolated naïve B6 CD4+ T cells, or in CD4+ T cells that were activated for 2 days in the presence of DMSO vehicle or varying concentrations of trametinib. FIG. 12B shows dilution of CTV (left panel), which indicates proliferation of CD4+ T cells that were activated for 3 days in the presence of DMSO or 100 nM trametinib. Quantification of the results (right panel) is presented as division index. FIG. 12C shows contour plots (top two rows) and bar graphs (bottom row) which display frequencies of IFN-γ, IL-17, and Foxp3 expressing cells in CD4+ T cells that were cultured under Th1, Th17, and inducible Treg (iTreg) polarizing conditions for 3 days in the presence of DMSO or 100 nM trametinib. In another experiment, 10-week-old B6 female mice subjected to MOG35-55-induced EAE were treated with corn oil or 3 mg/kg Trametinib every other day from day 0 to day 12 post immunization. FIG. 12D shows clinical scores of mice in each group. FIG. 12E shows contour plots (6 top panels) which display the frequency of CD4+TCRβ+ T cells among CD45+ cells in the brain tissues at 18-20 days post induction of EAE, and expression of GM-CSF, IL-17A, IFN-γ by those CD4+ T cells. Bar graphs (4 bottom panels of FIG. 12E) indicate the number of CD4+ T cells in the brain tissues, and frequencies of IFN-γ+, IL-17A+, GM-CSF+ cells among them. FIG. 12F shows percentage Balb/c heart allograft survival in B6 recipients that were treated with Trametinib or corn oil every other day from day 0 to day 12 post-transplant. FIG. 12G shows results of an experiment in which CD45.1+ mice were transferred with 5×10⁶ CD45.2+WT TEa cells on day −1, received Balb/c heart transplants on day 0 and treated with corn oil or 3 mg/kg Trametinib on days 0, 2, 4, and 6, followed by analysis of splenocytes on day 7. Dot plots of FIG. 12G show co-expression of CD45.2 with PD-1 (top row) or Helios (bottom row), gated on CD4+ cells. Bar graphs of FIG. 12G display PD-1 MFI and percent Helios+ cells of transferred CD45.2+ TEa cells. Data are mean±SD (FIGS. 12A-12C, 102E, and 12G) and are representative of two to three independent experiments. **P<0.01; unpaired student's t-test (FIGS. 12A, 12B, 12C, 12E, and 12G); Mann-Whitney test (FIGS. 12D and 12F).

FIGS. 13A through 13E show effects of Trametinib treatment on T cells in vitro. In FIG. 13A and FIG. 13B, naïve B6 CD4+ T cells were activated for 2 days in the presence of indicated cytokines or inhibitors, followed by western blot and flow cytometry analysis. FIG. 13A shows Western blots showing the expression of BATF and IRF4. FIG. 13B shows a bar graph which illustrates the IRF4 MFI of WT CD4+ T cells. In FIG. 13C and FIG. 13D, naïve B6 CD4+ T cells were activated for 2 days in the presence of indicated concentrations of trametinib. FIG. 13C shows representative contour plots (top) and a bar graph (bottom) which display the frequency of live-cell populations (Zombie Aqua negative) in cultured CD4+ T cells. FIG. 13D depicts representative contour plots showing IRF4 expressions in activated CD4+ T cells. In FIG. 13E, naïve B6 or Irf4^{fl/fl}Cd4-Cre CD4+ T cells were activated for 2 days with or without trametinib treatment. Contour plots (top) and the bar graph (bottom) display the expression of PD-1 and Helios in activated CD4 T cells. *P<0.05 (unpaired student's t-test). Data in FIG. 13C and FIG. 13E are mean±SD.

FIGS. 14A through 14C show effects of Trametinib treatment on T cells in experimental autoimmune encephalomyelitis (EAE). B6 mice were subjected to EAE induction and treated with 3 mg/kg Trametinib or corn oil every other day from day 0 to day 12. FIG. 14A shows the gating strategy for flow cytometry analysis of CD4+ T cells in the brain tissue. FIG. 14B shows representative contour plots (top panels) and a bar graph (bottom panel) which show the frequency of Foxp3+ cells among CD4+ T cells in the brain tissue at 18-20 days post induction of EAE. FIG. 14C shows representative contour plots and bar graphs which indicate the frequency of IFN-γ+, IL-17A+, GM-CSF+, and Foxp3+ cells among CD4+ T cells in the draining lymph nodes (LN) and spleens at 18-20 days post induction of EAE. **P<0.01 (unpaired student's t-test). Data are mean±SD.

FIGS. 15A through 15C show IRF4 deletion in T cells induces transplant tolerance in heart graft recipients. Irf4^{fl/fl}Cd4-Cre mice were transplanted with BALB/c hearts. Thirty days later, recipients were transplanted again with BALB/c and C3H skins (n=5). FIG. 15A is a graph showing the percentage of skin allograft survival after skin transplantation on BALB/c heart-transplanted Irf4^{fl/fl}Cd4-Cre recipients. **P=0.0079; Mann-Whitney test. FIG. 15B shows representative images of accepted BALB/c skin allografts (>100 days) on BALB/c heart-transplanted Irf4^{fl/fl}Cd4-Cre recipients. FIG. 15C shows representative images of C3H (left) and BALB/c (right) skin allografts on a BALB/c heart-transplanted Irf4^{fl/fl}Cd4-Cre recipient.

FIGS. 16A through 16C show adoptive transfer of IRF4 re-introduced Irf4^{-/-} CD4⁺ T cells breaks transplant tolerance in Irf4-decificient mice. Irf4^{-/-} CD4⁺ T cells were stimulated with allogenic BALB/c splenic DCs and IL-2 for 3 days, followed by transduction with IRF4-GFP or GFP-Ctrl retrovirus for 1 day. Irf4^{fl/fl}Cd4-Cre mice were transplanted with BALB/c hearts and adoptively transferred with one million IRF4-GFP or GFP-Ctrl transduced Irf4^{fl/fl} CD4⁺ T cells. Thirty days later, recipient mice in the IRF4-GFP group were transplanted again with BALB/c skins (n=4), whereas recipients in the GFP-Ctrl group were transplanted with both C3H and BALB/c skins (n=4). FIG. 16A is a graph showing the percentage of skin allograft survival after skin transplantation on BALB/c heart-transplanted Irf4^{fl/fl}Cd4-Cre recipients that had been adoptively transferred with IRF4-GFP or GFP-Ctrl transduced Irf4^{fl/fl} CD4⁺ T cells. FIG. 16B shows representative images of rejected (left 3 panels) and accepted (right 3 panels) BALB/c skins on BALB/c heart transplanted Irf4^{fl/fl} Cd4-Cre recipients that had been adoptively transferred with IRF4-GFP and GFP-Ctrl transduced Irf4^{fl/fl} CD4⁺ T cells, respectively. FIG. 16C shows representative images of C3H (left) and BALB/c (right) skin allografts on a BALB/c heart-transplanted Irf4^{fl/fl}Cd4-Cre mouse that had been adoptively transferred with GFP-Ctrl transduced Irf4^{-/-} CD4⁺ T cells.

FIGS. 17A through 17C show checkpoint blockade does not prevent the establishment of transplant tolerance in Irf4-deficient mice. Irf4^{fl/fl}Cd4-Cre mice were transplanted with BALB/c hearts on day 0 and treated with anti-PD-L1 and anti-CTLA-4 (αPD-L1+αCTLA-4) mAbs on days 0, 3, and 5 to trigger heart graft rejection. Recipients with rejected heart allografts were then transplanted again with BALB/c and C3H skin allografts 30 days after heart grafting (n=6). FIG. 17A is a graph showing the percentage of skin allograft survival after skin transplantation on αPD-L1+αCTLA-4 treated, BALB/c heart graft rejected Irf4^{fl/fl}Cd4-Cre mice. **P=0.0022; Mann-Whitney test. FIG. 17B shows representative images of accepted BALB/c skin allografts (>100 days) on αPD-L1+αCTLA-4 treated, BALB/c heart graft rejected Irf4^{fl/fl}Cd4-Cre recipients. FIG. 17C shows representative images of C3H (left) and BALB/c (right) skin allografts on a αPD-L1+αCTLA-4 treated, BALB/c heart graft rejected Irf4^{fl/fl}Cd4-Cre mouse.

FIGS. 18A through 18C show identification of un-restored genes in alloreactive Irf4^{-/-} CD4⁺ T cells upon checkpoint blockade. CD45.1⁺ B6 mice were adoptively transferred with CD45.2⁺ WT or Irf4^{-/-} TEa cells on day −1, and transplanted with BALB/c hearts on day 0. Recipients transferred with Irf4^{-/-} TEa cells were further treated with rat IgG or anti-PD-L1 plus anti-CTLA-4 mAbs (P+C group) on days 0, 3, and 5. Adoptively transferred CD45.2⁺ TEa cells were isolated from splenocytes on day 6 by flow cytometry sorting. RNA was isolated for microarray analysis. FIG. 18A is a schematic of the experimental design. Heat maps in FIGS. 18B and 18C show the normalized gene expression scores from indicated groups. FIG. 18B shows differentially expressed genes between adoptively transferred WT TEa and Irf4^{-/-} TEa cells (IgG group). FIG. 18C shows selected unrestored genes in Irf4^{-/-} TEa cells following checkpoint blockade. Two RNA samples of each group were obtained from two independent experiments. Each RNA sample was isolated from pooled TEa cells from three (WT TEa group and Irf4^{-/-} TEa P+C group) or five (Irf4^{-/-} TEa IgG group) recipient mice.

FIGS. 19A through 19F show the checkpoint blockade does not restore effector memory cell generation from alloreactive Irf4^{-/-} CD4⁺ T cells. CD45.1⁺ B6 mice were adoptively transferred with mixed splenocytes containing a 1:1 ratio of CD45.1⁺ CD45.2⁺ WT TEa and CD45.2⁺ Irf4^{-/-} TEa cells on day −1, transplanted with BALB/c hearts on day 0, and left untreated (FIG. 19A-C) or treated with αPD-L1+αCTLA-4 (FIGS. 19D-F) on days 0, 3, and 5. FIG. 19A, Schematic of the experimental design. FIG. 19B, Flow cytometry plots display the gating strategy detecting co-transferred CD45.1⁺ CD45.2⁺ TCR Vβ6⁺ WT TEa and CD45.1⁻ CD45.2⁺ TCR Vβ6⁺ Irf4^{-/-} TEa cells in peripheral blood at one week post-grafting. The line graph shows WT Tea and Irf4^{-/-} TEa cell frequencies in peripheral blood weekly after transplantation. FIG. 19C, Splenocytes were analyzed on day 30 post-grafting. Shown are the gating strategy detecting TEa cell populations, and the percentages of CD62L⁻ CD44⁺ and IFN-γ⁺ TNF-α^{hi} cells within WT TEa and Irf4^{-/-} TEa cell populations. FIG. 19D, Schematic of the experimental design, with αPD-L1+αCTLA-4 treatment. FIG. 19E, WT Tea and Irf4^{-/-} TEa cell frequencies in peripheral blood at one week post-grafting (flow cytometry plots) and weekly after transplantation (line graph). FIG. 19F, The percentages of CD62L⁻CD44⁺ and IFN-γ⁺TNF-α^{hi} cells within WT TEa and Irf4^{-/-} TEa cells in spleens at day 30 post-grafting. Data are mean±SD (n=3). *, P<0.05.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

### Terminology

Unless defined otherwise, all technical and scientific terms used herein have the same meaning as commonly understood to one of ordinary skill in the art to which this invention belongs. The following definitions are provided for the full understanding of terms used in this specification.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular cell type is disclosed and discussed and a number of modifications that can be made to the cell type are discussed, specifically contemplated is each and every combination and permutation of the cell type and the modifications that are possible unless specifically indicated to the contrary. Thus, if a class of cell types A, B, and C are disclosed as well as a class of cell types D, E, and F and an example of a combination cell type, or, for example, a combination cell type comprising A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is understood that the compositions disclosed herein have certain functions. Disclosed herein are certain structural requirements for performing the disclosed functions, and it is understood that there are a variety of structures which can perform the same function which are related to the disclosed structures, and that these structures will ultimately achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

As used in the specification and claims, the singular form “a,” “an,” and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

The terms “about” and “approximately” are defined as being “close to” as understood by one of ordinary skill in the art. In some non-limiting embodiments, the terms are defined to be within 10% of the associated value provided. In some non-limiting embodiments, the terms are defined to be within 5%. In still other non-limiting embodiments, the terms are defined to be within 1%.

Grammatical variations of “administer,” “administration,” and “administering” to a subject include any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time, overlapping in time, or one following the other. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

“Comprising” is intended to mean that the compositions, methods, etc. include the recited elements, but do not exclude others. It is expressly understood that where the compositions, systems, or methods use the term comprising, the specification also discloses the same compositions, systems, or methods using the terms “consisting essentially of” and “consisting of” as it relates to the modified elements.

“Identical” or percent “identity,” in the context of two or more nucleic acids or polypeptide sequences, refer to two or more sequences or subsequences that are the same or have a specified percentage of amino acid residues or nucleotides that are the same (e.g., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

“Pharmaceutically acceptable” component can refer to a component that is not biologically or otherwise undesirable, e.g., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Polynucleotide” and “oligonucleotide” are used interchangeably, and refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. A polynucleotide is composed of a specific sequence of four nucleotide bases: adenine (A); cytosine (C); guanine (G); thymine (T); and uracil (U) for thymine (T) when the polynucleotide is RNA. Thus, the term “polynucleotide sequence” is the alphabetical representation of a polynucleotide molecule.

“Peptide,” “protein,” and “polypeptide” are used interchangeably to refer to a natural or synthetic molecule comprising two or more amino acids linked by the carboxyl group of one amino acid to the alpha amino group of another. The amino acids may be natural or synthetic, and can contain chemical modifications such as disulfide bridges, substitution of radioisotopes, phosphorylation, substrate chelation (e.g., chelation of iron or copper atoms), glycosylation, acetylation, formylation, amidation, biotinylation, and a wide range of other modifications. A polypeptide may be attached to other molecules, for instance molecules required for function. Examples of molecules which may be attached to a polypeptide include, without limitation, cofactors, polynucleotides, lipids, metal ions, phosphate, etc. Non-limiting examples of polypeptides include peptide fragments, denatured/unstructured polypeptides, polypeptides having quaternary or aggregated structures, etc. There is expressly no requirement that a polypeptide must contain an intended function; a polypeptide can be functional, non-functional, function for unexpected/unintended purposes, or have unknown function. A polypeptide is comprised of approximately twenty, standard naturally occurring amino acids, although natural and synthetic amino acids which are not members of the standard twenty amino acids may also be used. The standard twenty amino acids include alanine (Ala, A), arginine (Arg, R), asparagine (Asn, N), aspartic acid (Asp, D), cysteine (Cys, C), glutamine (Gin, Q), glutamic acid (Glu, E), glycine (Gly, G), histidine, (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V). The terms “polypeptide sequence” or “amino acid sequence” are an alphabetical representation of a polypeptide molecule.

Conservative substitutions of amino acids in proteins and polypeptides are known in the art. For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gin; Ser, Thr; Lys, Arg; and Phe, Tyr. Such conservatively substituted variations of each explicitly disclosed sequence are included within the polypeptides provided herein.

Substantial changes in protein function or immunological identity are made by selecting substitutions that are less conservative, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

A “derivative” of a protein or peptide can contain post-translational modifications (such as covalently linked carbohydrate), depending on the necessity of such modifications for the performance of a specific function.

A “variant” refers to a molecule substantially similar in structure and immunoreactivity. Thus, provided that two molecules possess a common immunoactivity and can substitute for each other, they are considered “variants” as that term is used herein even if the composition or secondary, tertiary, or quaternary structure of one of the molecules is not identical to that found in the other, or if the amino acid or nucleotide sequence is not identical. Thus, in one embodiment, a variant refers to a protein whose amino acid sequence is similar to a reference amino acid sequence, but does not have 100% identity with the respective reference sequence. The variant protein has an altered sequence in which one or more of the amino acids in the reference sequence is deleted or substituted, or one or more amino acids are inserted into the sequence of the reference amino acid sequence. As a result of the alterations, the variant protein has an amino acid sequence which is at least 30%, 40%, 50%, 60%, 70%, 75%, 80%, 85%, 90%, or 95% identical to the reference sequence. For example, variant sequences which are at least 95% identical have no more than 5 alterations, i.e. any combination of deletions, insertions or substitutions, per 100 amino acids of the reference sequence. Percent identity is determined by comparing the amino acid sequence of the variant with the reference sequence using any available sequence alignment program. An example includes the MEGALIGN project in the DNA STAR program. Sequences are aligned for identity calculations using the method of the software basic local alignment search tool in the BLAST network service (the National Center for Biotechnology Information, Bethesda, Md.) which employs the method of Altschul, S. F., Gish, W., Miller, W., Myers, E. W. & Lipman, D. J. (1990) J. Mol. Biol. 215, 403-410. Identities are calculated by the Align program (DNAstar, Inc.) In all cases, internal gaps and amino acid insertions in the candidate sequence as aligned are not ignored when making the identity calculation.

“Specifically binds” when referring to a polypeptide (including TCRs and antibodies), refers to a binding reaction which is determinative of the presence of the protein or polypeptide or receptor in a heterogeneous population of proteins and/or other biologics. Thus, under designated conditions (e.g. immunoassay conditions), a specified receptor “specifically binds” to its particular “target” (e.g. a TCR specifically binds to an antigen) when it does not bind in a significant amount to other antigens present in the sample or to other biological components to which the ligand or antibody may come in contact in an organism. Generally, a first molecule (e.g., TCR) that “specifically binds” a second molecule (e.g., antigen) has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

“Therapeutically effective amount” or “therapeutically effective dose” of a composition (e.g. a composition comprising an agent) refers to an amount that is effective to achieve a desired therapeutic result. In some embodiments, a desired therapeutic result is reduced T-cell infiltration in a transplant. In some embodiments, a desired therapeutic result is transplant acceptance by the recipient. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The terms “treat,” “treating,” “treatment,” and grammatical variations thereof as used herein, include partially or completely delaying, curing, healing, alleviating, relieving, altering, remedying, ameliorating, improving, stabilizing, mitigating, and/or reducing the intensity or frequency of one or more diseases or conditions, symptoms of a disease or condition, or underlying causes of a disease or condition. Treatments according to the invention may be applied prophylactically, pallatively or remedially. Prophylactic treatments are administered to a subject prior to onset (e.g., before obvious signs) or during early onset (e.g., upon initial signs and symptoms). Prophylactic administration can occur for several days to years prior to the manifestation of symptoms.

In some instances, the terms “treat”, “treating”, “treatment” and grammatical variations thereof, include reducing T-cell infiltration in a transplant. The terms “treat”, “treating”, “treatment” and grammatical variations thereof, can also include reducing inflammation in a transplant, for example as measured by markers of inflammation such as cytokines, as understood by one of skill in the art. The terms “treat”, “treating”, “treatment” and grammatical variations thereof, can also include increasing the recipient's acceptance of a transplant, as determinable from absence of or low-level anti-transplant immune responses over time. The terms “treat”, “treating”, “treatment” and grammatical variations thereof, can also include reducing an autoimmune response, for example reducing T-cell responses to self-myelin. The terms “treat”, “treating”, “treatment” and grammatical variations thereof, can also include improving sensory and motor function in individuals suffering from an autoimmune disorder. Measurements of treatment can be compared with prior treatment(s) of the subject, inclusive of no treatment, or compared with the incidence of such symptom(s) in a general or study population.

### Methods to Improve Transplant Outcomes

The methods disclosed herein can be used to inhibit Interferon Regulatory Factor 4 (IRF4) in T-cells. The methods are useful for instances in which inhibited IRF4 in T-cells would be beneficial, for example in instances in which increased T-cell dysfunction would be advantageous. As such, the methods are useful at least for improving tissue and/or organ transplant outcomes.

Disclosed herein are methods to improve a transplant outcome in a recipient of a transplant comprising inhibiting IRF4 in T-cells of the recipient, thereby improving the transplant outcome. The methods are advantageous at least because they can be used to improve an array of measurable graft or transplant (collectively referred to herein as “transplant”) outcomes. For example, the methods can decrease expression of certain pro-inflammatory mediators or generally decrease local inflammation, reduce T-cell infiltration in a transplant, or increase or prolong a recipient's acceptance of a transplant, among other desirable transplant outcomes. T-cells are important mediators of transplant rejection, in part by effecting an immune response against the transplant, which can be deemed foreign by cells of the immune system. By inhibiting IRF4, a transcription factor involved in T cell function, transplant outcomes can be improved in part due to reduced T-cell mediated responses against the transplant. Without limitation or wish to be bound by any particular theory, it is believed that inhibition of IRF4 decreases T-cell functionality, thereby reducing T-cell mediated attack on a transplant, even a partially or fully MHC-mismatched transplant.

The terms “transplant” and “graft” are used interchangeably herein and refer to any cell, tissue, or organ provided by a donor to a recipient. For example, the transplant can be comprised within a bodily fluid (e.g., blood cells within a blood transfusion) or can be a tissue transplant (e.g., a skin graft). Alternatively, the transplant can be an organ or a portion thereof (e.g., a heart transplant, or a pediatric transplant of a portion of an adult liver). The donor and the recipient can be the same (e.g., grafting the recipient's healthy skin to an area of burn or abrasion), but it is understood that the methods are highly advantageous when the donor and recipient are separate subjects.

“IRF4” refers to Interferon Regulatory Factor 4 (IRF4) polypeptide also known as IRF-4 and previously known as MUM1 and LSIRF and, in humans, is encoded by the IRF4 gene. In some embodiments, the IRF4 polypeptide or polynucleotide is that identified in one or more publicly available databases as follows: HGNC: 6119, Entrez Gene: 3662, Ensembl: ENSG00000137265, OMIM: 601900, and UniProtKB: Q15306. The IRF4 polypeptide can be from any vertebrate, particularly from any mammal, for instance livestock such as cows, pigs, and sheep, primates such as humans, gorillas and monkeys, rodents such as mice, rats and guinea pigs, and other mammals such as horse, dog, bear, deer, dolphin, felines, etc. In some embodiments, the IRF4 polypeptide is a human IRF4 polypeptide, or at least a portion of a human IRF4 polypeptide. In some embodiments, the IRF4 polypeptide may be a chimeric polypeptide comprising at least a portion of a human IRF4 polypeptide and a portion of an IRF4 polypeptide from another species or a synthetic source. Example IRF4 polypeptides can include, for example, the following sequences as identified by their accession numbers: Human [Homo sapiens] IRF4 isoform 1, NCBI Reference Sequence: NP_002451.2, GI: 167555104; Human IRF4 isoform 2, NCBI Reference Sequence: NP_001182215.1, GI: 305632828; House mouse [Mus musculus] IRF4 isoform a, NCBI Reference Sequence: NP_038702.1, GI: 7305519; House mouse IRF4 isoform b, NCBI Reference Sequence: NP_001334437.1, GI: 1109303185; The brown rat [Rattus norvegicus] IRF4, NCBI Reference Sequence: NP_001099578.1, GI: 157816963; Chinese hamster [Cricetulus griseus] IRF4, GenBank: RLQ69839.1, GI: 1494136450; Sheep [Ovis aries] IRF4, NCBI Reference Sequence: XP_027814722.1, GI: 1567534866; Cattle [Bos taurus] IRF4, NCBI Reference Sequence: NP_001193091.1, GI: 329663890.

In some embodiments, the IRF4 is a polypeptide comprising an amino acid sequence which is at least 80% identical to SEQ ID NO: 1. In some embodiments, the IRF4 is a polypeptide comprising an amino acid sequence which is at least 85%, at least 90%, at least 95%, at least 98%, or at least 99% identical to SEQ ID NO: 1. In some embodiments, the IRF4 is a polypeptide comprising comprises SEQ ID NO: 1.

In some embodiments, the IRF4 is a polynucleotide comprising a nucleic acid sequence which is at least 70% identical to SEQ ID NO: 2. In some embodiments, the IRF4 is a polynucleotide comprising a nucleic acid sequence which is at least 75%, at least 80%, at least 85%, at least 90%, at least 95%, at least 98%, or at least 99% identical to SEQ ID NO: 2. In some embodiments, the IRF4 is a polynucleotide comprising the nucleic acid sequence of SEQ ID NO: 2.

In some embodiments, the IRF4 is a polypeptide comprising an amino acid sequence which is at least 80% identical to SEQ ID NO: 26. In some embodiments, the IRF4 is a polypeptide comprising an amino acid sequence which is at least 85%, at least 90%, at least 95%, at least 98%, or at least 99% identical to SEQ ID NO: 26. In some embodiments, the IRF4 is a polypeptide comprising comprises SEQ ID NO: 26.

In some embodiments, the IRF4 is a polynucleotide comprising a nucleic acid sequence which is at least 70% identical to SEQ ID NO: 25. In some embodiments, the IRF4 is a polynucleotide comprising a nucleic acid sequence which is at least 75%, at least 80%, at least 85%, at least 90%, at least 95%, at least 98%, or at least 99% identical to SEQ ID NO: 25. In some embodiments, the IRF4 is a polynucleotide comprising the nucleic acid sequence of SEQ ID NO: 25.

In some embodiments, the IRF4 is a polynucleotide comprising a nucleic acid sequence which is at least 70% identical to SEQ ID NO: 27. In some embodiments, the IRF4 is a polynucleotide comprising a nucleic acid sequence which is at least 75%, at least 80%, at least 85%, at least 90%, at least 95%, at least 98%, or at least 99% identical to SEQ ID NO: 27. In some embodiments, the IRF4 is a polynucleotide comprising the nucleic acid sequence of SEQ ID NO: 27.

In some embodiments, the IRF4 is a polynucleotide comprising a nucleic acid sequence which is at least 70% identical to SEQ ID NO: 28. In some embodiments, the IRF4 is a polynucleotide comprising a nucleic acid sequence which is at least 75%, at least 80%, at least 85%, at least 90%, at least 95%, at least 98%, or at least 99% identical to SEQ ID NO: 28. In some embodiments, the IRF4 is a polynucleotide comprising the nucleic acid sequence of SEQ ID NO: 28.

The disclosed methods include inhibiting IRF4 such that a transplant outcome is improved. Generally, the degree of IRF4 inhibition, the amount of T-cells in which IRF4 is inhibited, and the duration of inhibition should be sufficient so as to achieve an improvement in a transplant outcome.

Inhibition of IRF4 can be determined by measurement of IRF4 expression in T-cells of the recipient. The IRF4 expression measurements are typically compared to a control. In some embodiments, the T-cells of the recipient have at least 50% decreased IRF4 expression compared to a control. In some embodiments, the T-cells of the recipient have at least 60%, at least 70%, at least 80%, at least 90%, at least 95%, at least 98%, or at least 99% decreased IRF4 expression compared to a control. Thus, some embodiments of the methods can comprise inhibiting IRF4 by reducing IRF4 expression by any of these amounts, as compared to a control.

IRF4 expression in T-cells of the recipient can be determined at the transcriptional level, the translational level, or combinations thereof, and can be measured via a wide array of methods used to measure gene or polypeptide expression levels. In some embodiments, IRF4 expression can be measured at the gene transcription level. For example and without limitation, levels of IRF4 mRNA transcripts can be determined by radiation absorbance (e.g., ultraviolet light absorption at 260, 280, or 230 nm), quantification of fluorescent dye or tag emission (e.g., ethidium bromide intercalation), quantitative polymerase chain reaction (qPCR) of cDNA produced from mRNA transcripts, southern blot analysis, gene expression microarray, or other suitable methods. Increased levels of mRNA transcripts can be used to infer or estimate increased levels of polypeptide expression. In some embodiments, IRF4 expression can be measured at the post-translational level. For example and without limitation, levels of IRF4 polypeptide can be determined by radiation absorbance (e.g., ultraviolet light), bicinchoninic acid (BCA) assay, Bradford assay, biuret test, Lowry method, Coomassie-blue staining, functional or enzymatic assay, immunodetection and/or Western blot analysis, or other suitable methods.

Inhibition of IRF4 can also be determined by measuring IRF4 functionality in T-cells of the recipient compared to a control. Thus, the methods include some or further embodiments in which expression levels of IRF4 may or may not be altered compared to a control, but the function of IRF4 is reduced compared to a control. In some embodiments, the T-cells of the recipient have at least 50% decreased IRF4 functionality compared to a control. In some embodiments, the T-cells of the recipient have at least 60%, at least 70%, at least 80%, at least 90%, at least 95%, at least 98%, or at least 99% decreased IRF4 functionality compared to a control. Thus, some embodiments of the methods can comprise inhibiting IRF4 by reducing IRF4 functionality by any of these amounts, as compared to a control.

IRF4 functionality in T-cells of the recipient can be determined via a wide array of methods used to determine polynucleotide or polypeptide function, particularly IRF4 function. Although IRF4 has many known functions, the methods include inhibition of any one or more functions of IRF4, so long as the methods result in an improvement in a transplant outcome. In some embodiments, IRF4 functionality can be measured at the gene or gene transcript level. For example and without limitation, IRF4 polynucleotide can be evaluated for mRNA secondary structure (e.g., hairpin formation), DNA modifications (e.g., methylation, histone modification), binding of factors (e.g., transcription repressors, antisense RNA), functional and/or enzymatic assay (e.g., mRNA translation assays), presence of nucleic acid sequence mutations known to reduce function, each of which may inhibit or reduce the coding/translation function of the polynucleotide. In some embodiments, IRF4 functionality can be measured at the polypeptide level. For example and without limitation, IRF4 polypeptide functionality can be determined by secondary and/or tertiary folding analysis (e.g., incomplete or incorrect protein folding determined by circular dichroism, crystallography, nuclear magnetic resonance, electron microscopy, or other methods), IRF4 sequestration experiments (e.g., coimmunoprecipitation with a repressor or inhibitor), IRF4 polypeptide isolation and complementation of a cellular or in vitro functional assay (e.g., DNA-binding assay), presence of amino acid sequence mutations known to reduce function, or other suitable methods.

IRF4 can be inhibited for a time sufficient to achieve an improvement in a transplant outcome, and can be inhibited before, after, or both before and after transplantation. This metric can be adapted to the transplant outcome sought. For example, a shorter duration of IRF4 inhibition may be used to facilitate a short-term transplant outcome (e.g., initial transplant acceptance), whereas a longer duration of IRF4 inhibition may be used to achieve a long-term transplant outcome (e.g., permanent transplant acceptance). It is understood that the degree of IRF4 inhibition can vary throughout the duration of IRF4 inhibition.

Optionally, IRF4 can be inhibited in the T-cells of the recipient prior to transplantation. In some embodiments, IRF4 is inhibited for at least six hours or at least 12 hours before transplantation. In some embodiments, IRF4 is inhibited for at least one day, at least two days, at least three days, or at least four days before transplantation. In some embodiments, IRF4 is inhibited for at least one week, at least two weeks, at least three weeks, or at least four weeks before transplantation.

Optionally, IRF4 can be inhibited in the T-cells of the recipient subsequent to transplantation. In some embodiments, IRF4 is inhibited for at least six hours or at least 12 hours after transplantation. In some embodiments, IRF4 is inhibited for at least one day, at least two days, at least three days, or at least four days after transplantation. In some embodiments, IRF4 is inhibited for at least one week, at least two weeks, at least three weeks, or at least four weeks after transplantation. In some embodiments, IRF4 is inhibited for at least one month, at least two months, at least three months, or at least four months, at least six months, or at least nine months after transplantation. In some embodiments, IRF4 is inhibited for at least one year or longer after transplantation. In some embodiments, inhibition of IRF4 may be suspended after IRF4 is inhibited for any herein disclosed period of time, and after such suspension in IRF4 inhibition, IRF4 may be inhibited a second or more times. Optionally, IRF4 can be inhibited in the T-cells of the recipient both prior to and subsequent to transplantation.

The duration of IRF4 inhibition can proceed uninterrupted for a specified period of time, or can be intermittently interrupted by temporarily halting the method to inhibit IRF4 (e.g., withholding an IRF4 inhibitory treatment) or reducing the effectiveness of the method to inhibit IRF4 (e.g., reducing the amount of administered IRF4 inhibitory treatment). Such periods in which the methods are temporarily halted or the effects thereof are reduced can be used to reduce the potential for immunocompromization, or alternatively, to test whether continued IRF4 inhibition remains beneficial to facilitate maintenance of an improvement in transplant outcome. For example, long-term acceptance of a transplant can be tested by temporarily halting the disclosed method, thereby facilitating the increase in IRF4 expression or functionality in the T-cells of the recipient. Indicators such as absence of subsequent T-cell infiltration and/or T-cell mediated anti-transplant responses may indicate long-term acceptance of the transplant.

IRF4 is inhibited in the T-cells of the recipient, of which the level of inhibition typically can be determined by comparison to a control. The control can comprise a biological sample, or alternatively, a collection of values used as a standard applied to one or more subjects (e.g., a general number or average that is known and not identified in the method using a sample). In some embodiments, the control comprises an unmodified cell of the recipient (e.g., a baseline sample). An unmodified cell of the recipient can be obtained from the recipient prior to inhibiting IRF4. By “unmodified cell,” it is meant that the cell is obtained from a recipient, or from a biological sample of a recipient, and measured for IRF4 expression or functionality without additional steps or manipulations performed on the cell beyond those required to obtain the cell and measure IRF4 expression or functionality. For example, an unmodified cell may be obtained by a standard phlebotomy technique, centrifuged to remove blood or plasma liquid components, washed and resuspended in buffered solutions, and subjected to a polynucleotide or polypeptide measurement technique (e.g., the cell may be lysed, and the contents extracted and subjected to a Western blot analysis using an anti-IRF4 monoclonal antibody). A storage step can be included between the obtaining step and the IRF4 measuring step (e.g., in cryogenic conditions) for both the control and the recipient's T-cells having inhibited IRF4.

In some embodiments, the control comprises an unmodified T-cell of the recipient. In some embodiments, the control comprises an unmodified T-cell of the recipient that is the same T-cell type as the recipient's T-cells having inhibited IRF4. For example and without limitation, the control can comprise an unmodified CD4+ T-cell of the recipient in embodiments comprising inhibiting IRF4 in CD4+ T-cells of the recipient. However, it is neither required that the control and the T-cells having inhibited IRF4 be of the same T-cell type, nor that the control and the T-cells having inhibited IRF4 be obtained from the recipient at the same or a similar time.

T-cells of the recipient can be obtained from the recipient by any means appropriate to recover T-cells for IRF4 inhibition analysis (e.g., expression and/or functionality analysis). For example, the T-cells can be obtained from a biological sample of the recipient. The biological sample can be any T-cell-containing biological sample, for example, blood, plasma, lymph, tissue, biopsy, and the like. The biological sample can be obtained by standard medical, clinical, and/or phlebotomy techniques, and the biological sample can be further processed as required (e.g., purification, culture, storage) in preparation for or in accompaniment with measuring IRF4 inhibition in the T-cells.

IRF4 can be inhibited in T-cells in a number of ways. In some embodiments, IRF4 can be inhibited primarily or specifically in T-cells of the recipient, or in a T-cell subtype of the recipient (e.g., CD4+ T-cells). For example, a T-cell-specific therapeutic can be administered or, alternatively, in vitro-manipulated T-cells can be adoptively transferred to the recipient. However, specific inhibition in only T-cells of the recipient is not expressly required, and methods to inhibit IRF4 in T-cells can include IRF4 inhibition in other cell types, provided that such inhibition is not substantively counterproductive to improving a transplant outcome facilitated by the disclosed methods or does not cause undesirable effects in the recipient which outweigh the benefits of the improved transplant outcome.

IRF4 can be directly inhibited or, alternatively, indirectly inhibited. Thus, the transplant outcomes can be improved if IRF4 is ultimately inhibited in at least one direct and/or indirect way. For example and without limitation, an agent may bind to and directly inhibit the IRF4 gene promoter or IRF4 polypeptide. Alternatively and without limitation, an agent may inhibit a positive regulator of IRF4 (e.g., a molecule which increases IRF4 expression or functionality), or may activate or increase expression of a negative regulator of IRF4 (e.g., a molecule which decreases IRF4 expression or functionality).

In some embodiments, IRF4 can be inhibited by administering to the recipient an IRF4 inhibitor. The IRF4 inhibitor can be any agent, compound, molecule, or other composition capable of IRF4 inhibition when administered to a recipient. In some embodiments, the IRF4 inhibitor comprises a pharmaceutical compound. In some embodiments, the IRF4 inhibitor comprises a MEK 1/2 inhibitor. In some embodiments, the IRF4 inhibitor comprises trametinib. In some embodiments, the IRF4 inhibitor comprises pomalidomide (Pomalyst). In some embodiments, the IRF4 inhibitor comprises an IRF4 RNA interference (RNAi) modulator. RNAi modulators can be used to silence the expression of a gene (also known as “gene knock-down”), as opposed to genetic disruption of the gene (also known as “gene knock out”). RNAi modulators include, but are not limited to, small interfering RNA (siRNA), short hairpin RNA (shRNA), micro RNA (mRNA), trans-acting small interfering RNA (tasiRNA), long non-coding RNA (lncRNA), Piwi-interacting RNA (piRNA), among others. In some embodiments, the IRF4 inhibitor comprises an anti-IRF4 siRNA. For example, see example sequences for IRF4 siRNAs [CCACAGAUCUAUCCGCCAU (SEQ ID NO:29), UGUCAGAGCUGCAAGCGUU (SEQ ID NO: 30), and GAAAAUGGUUGCCAGGUGA (SEQ ID NO:31)] from Cherian M A et al. J Biol Chem. 2018 May 4; 293(18):6844-6858, which is incorporated herein by reference.

In some embodiments, IRF4 can be inhibited in T-cells of the recipient by an in vitro method. In such embodiments, the T-cells having inhibited IRF4 expression or functionality may be adoptively transferred to the recipient. Such methods permit treatment on cells of the recipient in place of, or in addition to, treatment of the recipient (e.g., systemic administration of an IRF inhibitory agent). T-cells of the recipient for in vitro IRF4 inhibition can be obtained from the recipient by any means described herein. Further, IRF4 can be inhibited in the obtained T-cells by any of the disclosed methods, as applied to in vitro methods to inhibit IRF4. Alternatively, the IRF4 gene may be genetically interrupted, so as to knock out expression of IRF4 in the T-cells.

T-cells having in vitro inhibited IRF4 can be adoptively transferred to the recipient by an administering step, which can include any method of introducing the T-cells into the recipient appropriate for the T-cell formulation. Prior to administration, cells for adoptive transfer are typically purified or separated from other cells, for example by fluorescence activated cell sorting (FACS) or microfluidics methods. Cells can be further increased in number (e.g., via culturing) to obtain a sufficient amount of cells for adoptive transfer. The T-cells can be administered in a number of ways, for instance, as circulating T-cells (e.g., by intravenous injection) or implanted into a tissue (e.g., near a transplant). In some embodiments, the administering step comprises systemic administration (e.g., by intravenous injection). In some embodiments, the administering step comprises local administration, for example locally administered near a transplant or transplant site. In some embodiments, at least about 1,000 T-cells are administered. In some embodiments, at least about 10,000, at least about 100,000, at least about 500,000, at least about 1,000,000, at least about 5,000,000, at least about 10,000,000, at least about 50,000,000, or at least about 100,000,000 or more T-cells are administered.

The amount of IRF4 inhibitor or adoptively transferred T-cells administered to the recipient can vary widely, but should be sufficient to result in improvement of a transplant outcome. The amount of IRF4 inhibitor or adoptively transferred T-cells administered to the recipient can be expressed in terms of a dosage amount per body weight. The amount of the disclosed compositions administered to a recipient will vary from recipient to recipient, depending on the nature of the disclosed compositions and/or formulations, the species, gender, age, weight and general condition of the recipient, the mode of administration, and the like. Effective dosages and schedules for administering the compositions may be determined empirically, and making such determinations is within the skill in the art. The dosage ranges for the administration of the disclosed compositions are those large enough to produce the desired effect (e.g., to reduce T-cell infiltration in a transplant). The dosage should not be so large as to outweigh benefits by causing adverse side effects, such as unwanted cross-reactions, anaphylactic reactions, and the like. The dosage can be adjusted by the individual clinician in the event of any counterindications. Generally, the disclosed compositions and/or formulations are administered to the recipient at a dosage of active component(s) ranging from 0.1 μg/kg body weight to 100 g/kg body weight. In some embodiments, the disclosed compositions and/or formulations are administered to the recipient at a dosage of active component(s) ranging from 1 μg/kg to 10 g/kg, from 10 μg/kg to 1 g/kg, from 10 μg/kg to 500 mg/kg, from 10 μg/kg to 100 mg/kg, from 10 μg/kg to 10 mg/kg, from 10 μg/kg to 1 mg/kg, from 10 μg/kg to 500 μg/kg, or from 10 μg/kg to 100 μg/kg body weight. Dosages above or below the range cited above may be administered to the individual recipient if desired.

IRF4 inhibition typically comprises administering an IRF4 inhibitor or adoptively transferred T-cells having IRF4 inhibition. The methods can comprise at least one, at least two, at least three, at least four, at least five, at least six, at least seven, at least eight, at least nine, or at least ten dosages or administrations. The dosages or administrations can be performed before the recipient exhibits symptoms of a transplantation complication (e.g., prophylactically), or during or after symptoms of a transplant complication occur.

In some embodiments, a subsequent administration is provided at least one day after a prior administration, or at least two days, at least three days, at least four days, at least five days, or at least six days after a prior administration. In some embodiments, a subsequent administration is provided at least one week after a prior administration, or at least two weeks, at least three weeks, or at least four weeks after a prior administration. In some embodiments, a subsequent administration is provided at least one month, at least two months, at least three months, at least six months, or at least twelve months after a prior administration.

The methods can be performed with or without administration of additional agents (e.g., therapeutic agents, diagnostic agents). In some embodiments, the methods can include administering one or more additional therapeutics in addition to inhibiting IRF4 in the T-cells of the recipient. It is understood that methods can encompass any known additional therapeutic used for improving a transplant outcome, for example immunosuppressive and/or anti-inflammatory agents. Non-limiting examples of suitable therapeutics which can be used in the methods include corticosteroids such as prednisone, prednisolone, budesonide and hydrocortisone, calcineurin inhibitors such as ciclosporin and tacrolimus, anti-proliferatives such as azoathioprine, leflunomide, myophenylate mofetil and mycophenolic acid, mTOR inhibitors such as sirolimus and everolimus, anti-IL2 and anti-IL2Rα therapeutics such as basiliximab and daclizumab, anti-T-cell therapeutics such as anti-thymocyte globulin (ATG) and anti-lymphocyte globulin (ALG), anti-CD20 therapeutics such as rituximab, ocrelizumab, ofatumumab, and obinutuzumab, TNFα inhibitors such as etanercept, infliximab, golimumab, adalimumab, and certolizumabor other immunosuppressive and/or anti-inflammatory therapeutics such as muromonab-CD3 (Orthoclone OKT3), rapamycin, tocilizumab, methotrexate, lenalidomide, among others, and combinations thereof.

The T-cell types in which IRF4 can be inhibited include, for example, effector T-cells, helper T-cells, cytotoxic T-cells, memory T-cells, regulatory T-cells, gamma-delta T-cells, engineered T cells, chimeric antigen receptor (CAR) T cells, etc. In some or further embodiments, the T-cells are activated T-cells. In some embodiments, the T-cells comprise CD4+ T-cells, CD8+ T-cells, or combinations thereof. In some embodiments, the T-cells comprise CD4+ T-cells. CD4+ T-cells are also referred to as helper T-cells and can function to regulate immune responses. Thus, inhibiting IRF4 in CD4+ T-cells can, in some embodiments, result in favorable immune responses to the transplant, thereby facilitating overall outcomes of the transplantation (e.g., acceptance of the transplant).

The recipient can be any mammalian subject, for example a human, dog, cow, horse, mouse, rabbit, etc. which has an IRF4 gene. In some embodiments, the recipient is a primate, particularly a human. The recipient can be a male or female of any age, race, creed, ethnicity, socio-economic status, or other general classifiers.

Typically, the recipient receives a transplant from a donor of the same species, although the methods can also be performed in xenotransplantation. The recipient and donor can have identical MHC profiles (e.g., genetically identical twins) or similar MHC profiles (e.g., MHC profiles generally considered sufficiently similar under traditional donor-recipient matching criteria). In some embodiments, the recipient and donor can have mismatched MHC profiles. A mismatched MHC profile is defined as being generally considered sufficiently different under traditional donor-recipient matching criteria such that the recipient would not be recommended to receive a transplant from the donor, typically due to risk of triggering an alloimmune response.

MHC profiles can contain an array of genes and gene variants, but an important gene used for determining MHC profiles for the purpose of transplantation include the Human Leukocyte Antigens (HLA)-A, —B, and -DR. For example, a recipient MHC profile of HLA-A1/2, HLA-B7/8, and HLA-DR10/11 may be considered sufficiently similar to a donor MHC profile of HLA-A1/2, HLA-B7/8, and HLA-DR10/12, such that the recipient would be recommended to receive a transplant from the donor under traditional donor-recipient matching techniques, particularly if the recipient tests negative for crossmatch antibody against the donor's HLA subtypes. However, a recipient MHC profile of HLA-A1/2, HLA-B7/8, and HLA-DR10/11 may be considered sufficiently different to a donor MHC profile of HLA-A15/24, HLA-B13/B14, and HLA-DR5/17, such that the recipient would be considered to have a mismatched MHC profile compared to the donor and would not be recommended to receive a transplant from the donor under traditional donor-recipient matching techniques. In some embodiments, the recipient comprises a MHC profile which is fully mismatched compared to a donor of the transplant. The term “fully mismatched” as it relates to a MHC profile refers to a donor MHC profile and a recipient MHC profile which have no HLA-A, HLA-B, and HLA-DR subtypes in common.

The disclosed methods can be used to improve a transplant outcome in a transplant recipient. In some embodiments, the transplant outcome comprises reduced inflammation in the transplant. In some embodiments, the transplant outcome comprises reduced T-cell infiltration in the transplant. In some embodiments, the transplant outcome comprises reduced expansion of alloreactive T cells. Any metric of an improved transplant outcome can be compared to any herein disclosed control.

In some or further embodiments, the transplant outcome comprises acceptance of the transplant. Acceptance of a transplant can be determined by absence of a significant alloimmune response against the transplant. In some embodiments, successful weaning of the recipient from immunosuppression can indicate transplant acceptance. Of note, episodes of acute alloimmune response against the transplant do not necessarily indicate a lack of transplant acceptance in cases where the acute alloimmune response can be controlled by short-term, temporary use of immunosuppressive therapeutics, wherein administration of the immunosuppressive therapeutics can be tapered down or withdrawn shortly after the acute alloimmune response subsides to a controlled level. Transplant acceptance can be determined at specific points in time after transplantation, for example one week, one month, or one year after transplantation. In some embodiments, the transplant outcome comprises acceptance of the transplant by the recipient for at least 10 days, at least 30 days, at least 50 days, or at least 100 days. In some embodiments, the transplant outcome comprises acceptance of the transplant by the recipient for at least 6 months, at least 9 months, at least 1 year, at least 3 years, at least 5 years, or at least 10 years.

In some embodiments, the improved transplant outcome can be indicated by cellular and/or molecular factors which indicate immunosuppression and/or anti-inflammation. For example, in some embodiments, the improved transplant outcome can be indicated by reduced Th1 and Th17 cell differentiation. In some embodiments, the improved transplant outcome can be indicated by reduced expression of effector T cell markers or signature cytokines for effector T cells. For example, the improved transplant outcome can be indicated by reduced expression by the T cells of CD44, and/or reduced frequencies of IFN-γ or IL-17 producing cells within the T cell population. In some embodiments, the improved transplant outcome can be indicated by increased expression of Ikzf2 (encoding Helios), Pdcd1 (encoding PD-1) or Cd160. In some embodiments, the improved transplant outcome can be indicated by an active chromatin state at the cis-elements of Pdcd1.

An IRF4 inhibitor, or adoptively transferred T-cells having IRF4 inhibition, administered to the recipient can be formulated with a pharmaceutically acceptable carrier and/or as a medicament. Suitable carriers include, but are not limited to, salts, diluents, binders, fillers, solubilizers, disintegrants, preservatives, sorbents, and other components.

Also disclosed herein are methods for establishing and/or promoting transplant tolerance in a recipient of a transplant comprising inhibiting IRF4 in T-cells of the recipient, thereby establishing and/or promoting transplant tolerance.

### Methods of Treating Myelination Disorders

Also disclosed herein are methods of treating a subject with a myelination disorder comprising inhibiting IRF4. Myelination disorders are autoimmune disorders which typically involve demyelination of nerves. Such disorders result in damage to or loss of the myelin protective coating or sheath which encase nerve. In some embodiments, the methods result in reduced T cell infiltration in the brain. In some embodiments, the methods result in improved motor function or reduced paralysis.

Any herein disclosed method to inhibit IRF4 (e.g., administration of an IRF4 inhibitor; adoptive transfer of T-cells having inhibited IRF4) can be used. Similarly, any herein disclosed control, T-cell type, additional therapeutics, methods to measure IRF4 inhibition, and treatment regimens can be included in the methods, as adapted to methods of treating a subject with a myelination disorder.

The methods are useful for treating myelination disorders which result from autoimmunity (e.g., demyelinating myelinoclastic disorders) such as optic neuritis, neuromyelitis optica, and transverse myelitis. In some embodiments, the myelination disorder comprises multiple sclerosis. In some embodiments, the myelination disorder comprises encephalomyelitis.

In some embodiments, IRF can be inhibited by administering an IRF4 inhibitor to the subject. In some embodiments, the IRF4 inhibitor comprises a MEK 1/2 inhibitor. In some embodiments, the IRF4 inhibitor comprises trametinib. In some embodiments, the IRF4 inhibitor comprises an anti-IRF4 siRNA. In some embodiments, the IRF4 inhibitor comprises an anti-IRF4 antibody.

The subject can be any mammalian subject, for example a human, dog, cow, horse, mouse, rabbit, etc. which has an IRF4 gene. In some embodiments, the subject is a primate, particularly a human. The subject can be a male or female of any age, race, creed, ethnicity, socio-economic status, or other general classifiers.

### Methods of Using or Measuring IRF4 Inhibition

Also disclosed herein are methods of increasing T-cell dysfunction in a subject comprising inhibiting IRF4, wherein the subject has an autoimmune disease or is a recipient of a transplant.

Autoimmune diseases include, but are not limited to, type I diabetes, celiac disease, Addison's disease, rheumatoid arthritis, Graves' disease, Lupus erythematosus, vasculitis, thyroid disease, systemic lupus erythematosus, psoriasis, scleroderma, myasthenia gravis, rheumatism, Hashimoto's thyroiditis, Sjogren syndrome, thyroiditis, connective tissue disease, autoimmune hepatitis, antiphospholipid syndrome, hemolytic anemia, bullous pemphigoid, myositis, primary biliary cholangitis, arteritis, pemphigus, Goodpasture syndrome, immune thrombocytopenic purpura, psoriatic arthritis, autoimmune polyendocrine syndrome, urticaria, polymyositis, vitiligo, autoimmune hemolytic anemia, alopecia areata, uveitis, myopathy, primary sclerosing cholangitis, among others.

T-cell dysfunction can be determined by in vivo and/or in vitro function assays. For example, T-cell dysfunction can be determined by absence of or reduced T-cell recruitment, infiltration, activation, proliferation, and/or secretion of pro-inflammatory cytokines.

Any herein disclosed method to inhibit IRF4 (e.g., administration of an IRF4 inhibitor; adoptive transfer of T-cells having inhibited IRF4) can be used, as adapted to methods of increasing T-cell dysfunction. Similarly, any herein disclosed control, T-cell type, additional therapeutics, methods to measure IRF4 inhibition, subjects, and treatment regimens can be included in the methods, as adapted to methods of increasing T-cell dysfunction.

Also disclosed herein are methods to identify a compound which inhibits IRF4 comprising contacting one or more T-cells with the compound; and measuring IRF4 expression in the one or more T-cells; wherein reduced IRF4 expression compared to a control indicates the compound inhibits IRF4; and wherein the control comprises one or more T-cells which are not contacted with the compound.

Also disclosed herein are methods to measure IRF4 expression in T-cells of a subject prescribed to receive a transplant comprising obtaining T-cells from the subject; and measuring IRF4 expression in the T-cells.

In some embodiments, wherein IRF4 expression in the T-cells is reduced compared to a control, the method further comprises advising the subject that the subject has an increased likelihood of accepting the graft. In some embodiments, wherein IRF4 expression in the T-cells is not reduced compared to a control, the method further comprises advising the subject that the subject does not have an increased likelihood of accepting the graft.

In some embodiments, wherein IRF4 expression in the T-cells is reduced compared to a control, the method further comprises administering to the subject an IRF4 inhibitor or adoptively transferring T-cells of the recipient having inhibited IRF4. In some such embodiments, the method can further comprise administering any one or more herein disclosed additional therapeutics.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended only as examples of the invention and do not limit the scope of what the inventors regard as their disclosure. These examples do not exclude equivalents and variations of the present invention which are apparent to one skilled in the art. Unless indicated otherwise, temperature is ° C. or is at ambient temperature, and pressure is at or near atmospheric. There are numerous variations and combinations of process conditions that can be used to optimize product quality and performance. Only reasonable and routine experimentation are required to optimize such process conditions.

### Example 1

### Materials and Methods

Mice. Cd4-Cre, Irf4^{flox/flox}, Rag1^{-/-}, B6.SJL CD45.1 congenic, TEa TCR transgenic, Foxp3gfp reporter, Balb/c, and C57BL/6 (B6) mice were purchased from Jackson Laboratory (Bar Harbor, Mass.). Irf4^{-/-} mice were previously described (Mittrucker et al., 1997; Ochiai et al., 2013). Irf4^{flox/flox} mice were crossed to Cd4-Cre mice to create Irf4^{fl/fl}Cd4-Cre mice. TEa mice were crossed to Irf4^{-/-} mice to create Irf4^{-/-} TEa mice. TCR transgenic (Vα2+Vβ6+) CD4+ T cells from TEa mice (B6 background; I-Ab, I-E-) are specific for Balb/c allopeptide I-Eα52-68 presented by B6 APCs and bound to I-Ab. Male mice at 8 to 10 weeks of age were used for heart transplantation, and 10-week-old B6 female mice were subjected to EAE induction. Littermates of the same sex were randomly assigned to experimental groups. Mice were housed in a specific pathogen free facility at Houston Methodist Research Institute in Houston, Tex. All animal experiments in this study were approved by the Houston Methodist Animal Care Committee in accordance with institutional animal care and use guidelines.

Flow cytometry. Splenocytes, lymph node cells, graft infiltrating cells (Chen et al., 2007), CNS-infiltrating cells, and cultured T cells were stained and analyzed on the LSR II flow cytometer (Beckton Dickinson), and the resulting data was processed by using FlowJo v10 software (Tree Star, Inc.). Fluorochrome-conjugated antibodies used for flow cytometry were as follows: specific for mouse CD3 (clone 145-201), TCRβ5 (H57-597), CD4 (GK1.5), CD8a (53-6.7), CD138 (281-2), GL7 (GL7), CD25 (PC61), CD62L (MEL-14), KLRG1 (MAFA), CD44 (IM7), CD45.1 (A20), CD45.2 (104), TCR Vα2 (B20.1), TCR Vβ56 (RR4-7), PD1 (29F.1A12), CD160 (7H1), Lag3 (C9B7W), CD73 (TY/11.8), FR4 (TH6), BTLA (6A6), PD-L1 (MIH5), Tim3 (B8.2C12), CD69 (H1.2F3), GITR (DTA), 4-1BB (17B5), 2B4 (m2B4 (B6)458.1), CD226 (480.1), CTLA-4 (UC10-4B9), ICOS (C398.4A), CD98 (RL388), CD71 (RI7217), TNF-α (MP6-XT22), IL-2 (JES6-5H4), IFN-γ (XMG1.2), IL-4 (11B11), IL-10 (JES5-16E3), IL-17A (TC11-18H10.1), IL-17F (9D3.1C8), Helios (22F6), CD45 (30-F11), Perforin (eBioOMAK-D), Granzyme B (GB11), BTLA (6A6), GM-CSF (MP1-22E9), IL-13 (eBio13A), CD19 (6D5), FoxP3 (FJK-16S), Ki67 (SolA15), BCL6 (K112-91), GLUT1, (SPM498), Fluorescein labeled Peanut Agglutinin (PNA), CXCR5 (2G8), APC Streptavidin, and BV421 Streptavidin. Dead cells were excluded from some analysis by using ZOMBIE AQUA™ Fixable Viability Kit (BioLegend). T cell proliferation was assessed by using the CELLTRACE™ Violet (CTV) Kit (Thermo Fisher Scientific). Intracellular expression of GLUT1, CTLA-4, and transcription factors were determined by using the Foxp3/Transcription Factor Staining Buffer Set (eBioscience) according to the manufacturers' instructions. For intracellular staining of cytokines, cultured or ex vivo isolated T cells were re-stimulated for 4 hours with 50 ng/ml phorbol 12-myristate 13-acetate (Sigma-Aldrich) and 500 ng/ml ionomycin (Sigma-Aldrich) in the presence of GolgiStop (BD Biosciences). Cells were fixed and permeabilized with CYTOFIX/CYTOPERM™ solution (BD Biosciences), followed by staining with fluorochrome-labeled antibodies against cytokines according to the manufacturers' instructions. For intracellular staining of IRF4, IRF4 antibody (M-17, goat polyclonal IgG) was purchased from Santa Cruz Biotechnology. Donkey anti-Goat IgG (H+L) Secondary Antibody conjugated with Alexa Fluor 488 or Alexa Fluor 647 was purchased from Thermo Fisher Scientific. T cells were fixed and permeabilized using the Foxp3/Transcription Factor Staining Buffer Set (eBioscience), and stained with IRF4 antibody and then with Donkey anti-Goat IgG (H+L) Secondary Antibody.

Murine heterotopic heart transplantation. Heart transplantation in mice was performed by a previously described method (Miyahara et al., 2012; Chen et al., 2007). In brief, hearts from Balb/c donors were transplanted into 8 to 10-week-old male WT B6, Irf4^{fl/fl}Cd4-Cre, CD45.1+ congenic, or Rag1−/− recipient mice. Pulmonary artery and aorta of donor heart were cut open, remaining heart vessels were tied off, and heart was removed. Anesthetized recipient mouse was opened by the midline incision and blood flow of the abdominal aorta and inferior vena cava was interrupted by ligation with 6-0 silk thread. Incisions were made in recipient abdominal aorta and inferior vena cava to perform anastomosis with donor heart aorta and donor pulmonary artery, respectively. Anastomosis was made by 11-0 sutures running continuously. 6-0 silk thread was removed, and abdomen was then closed by 5-0 sutures in two layers. Heart graft survival was monitored daily by palpation, and the day of complete cessation of heartbeat was considered as the day of rejection. Recipient mice were i.p. injected with Rat IgG, anti-PD-L1 (10F.9G2), anti-CTLA-4 (9D9), anti-CD4 (GK1.5), anti-CD8 (53-6.7), or anti-CD25 (PC-61.5.3) mAbs obtained from Bio-X-Cell (West Lebanon, N.H.), oral gavaged with trametinib (Selleck Chemicals, Houston, Tex.) or corn oil, or i.v. transferred with WT B6, Irf4^{-/-}, TEa, TEa Irf4^{-/-}, or IRF4 re-introduced Irf4^{-/-} T cells. At various days post-transplant, splenocytes and graft infiltrating cells were obtained for flow cytometry analysis, and allografts were sectioned and stained with hematoxylin and eosin for microscopic evaluation.

Reconstitution of Rag1^{-/-} mice with T cells. In the adoptive co-transfer model, Dynabeads Untouched Mouse T Cells Kit (Thermo Fisher Scientific) was used to isolate T cells from spleens of CD45.1+ congenic or Irf4^{-/-} mice. Rag1^{-/-} mice were co-injected i.v. with 2×10⁷ CD45.1+ congenic WT and 2×10⁷ CD45.2+(CD45.1−) Irf4^{-/-} T cells on day −1, and transplanted with Balb/c hearts on day 0. In the model transferred with separated T cell populations, 5×10⁶ FACS-sorted CD4+WT, CD4+ Irf4^{-/-}, CD8+WT, or CD8+ Irf4^{-/-} T cells were injected i.v. into B6.Rag1^{-/-} mice one day prior to BALB/c heart transplantation, respectively. Splenocytes and graft-infiltrating cells were obtained on day 9 for flow cytometry analysis.

Adoptive transfer of TEa transgenic T cells. TCR(Vα2+Vβ6+)CD4+ TEa cells were sorted from spleens of WT TEa or Irf4^{-/-} TEa mice by a FACSAria flow cytometer (BD Biosciences). B6.SJL CD45.1+ congenic mice were i.v. transferred with either 5×10⁶ CD45.2+WT TEa or 5×10⁶ CD45.2+ Irf4^{-/-} TEa cells on day −1, transplanted with Balb/c hearts or left un-transplanted on day 0, followed by flow cytometry analysis of TEa cells in spleens on day 6 or 7 as indicated in text. To assess TEa cell proliferation in response to heart transplantation, WT TEa or Irf4^{-/-} TEa cells were labeled CELLTRACE™ Violet prior to cell transfer. To determine effects of checkpoint blockade on M4^{-/-} TEa cells, CD45.1+ heart-transplanted recipients were transferred with CD45.2+ Irf4^{-/-} TEa cells and treated with either rat IgG or anti-PD-L1 plus anti-CTLA-4 mAbs. To determine effects of trametinib on WT TEa cells, CD45.1+ heart-transplanted recipients were transferred with CD45.2+WT TEa cells and treated with either corn oil or trametinib.

Induction and assessment of EAE. 10-week-old female B6 mice were subjected to EAE induction by using the HOOKE KIT™ MOG35-55/CFA Emulsion PTX (EK-0113; Hooke Laboratories, Lawrence, Mass.) according to the manufacturer's instructions, and were orally gavaged with corn oil or 3 mg/kg trametinib every other day from day 0 to day 12 post immunization. Mice were monitored daily for the development of clinical signs of EAE and scored according to the previously reported criteria (Lee et al., 2012). At 18-20 days post induction of EAE, mice were euthanized for flow cytometry analysis of T cells in CNS, spleen, and draining lymph nodes of the sites of immunization. Methods for isolation of CNS-infiltrating mononuclear cells have been previously reported (Lee et al., 2012; Xiao et al., 2016).

In vitro T cell stimulation. Naïve CD4+ T cells (CD62L+CD44− or CD62L+CD44−FoxP3GFP−) were sorted from WT B6, Irf4^{-/-}, or Foxp3gfp reporter mice by a FACSAria flow cytometer. B6 APCs were prepared by depletion of T cells from B6 splenocytes with phycoerythrin-anti-CD3 (clone 2C11; BioLegend) and anti-phycoerythrin microbeads (Miltenyi Biotec, San Diego, Calif.), followed by brief treatment with 50 μg/ml mitomycin C (Fisher Scientific) before each experiment. For activation of T cells, naïve CD4+ T cells were added in an amount of about 1×10⁵ cells/well in 96-well round bottom tissue-culture plates (Thermo Fisher Scientific), and stimulated with equal numbers of B6 APCs and 1 μg/ml soluble anti-CD3e mAh (clone 2C11; BioLegend). For some experiments, cell cultures were supplemented with various cytokines (PeproTech, Rocky Hill, N.J.) and small-molecule inhibitors (Selleck Chemicals). In some cases, naïve CD4+ T cells were labeled with CELLTRACE™ Violet reagent prior to stimulation. CD4+ T cells cultured for different days were collected and analyzed with flow cytometry, microarray analysis, Immunoblot, and quantitative real-time PCR, and ChIP.

Polarization of CD4+ T cells in vitro. For Th17 polarization, WT naïve CD4+ cells were activated for 3 days in the presence of 1 ng/ml human TGF-β1, 10 ng/ml mouse IL-6, 10 ng/ml IL-1β, 10 ng/ml IL-23, 5 μg/ml anti-IL-2 (JES6-1A12), 5 μg/ml anti-IL-4 (11B11), and 5 μg/ml anti-IFN-γ (XMG 1.2), followed by flow cytometry analysis of IL-17A and IL-17F expressions. For some experiments, cell cultures were supplemented with 0.2% DMSO or 100 nM trametinib. Recombinant cytokines were purchased from PeproTech and R&D systems, and cytokine-neutralizing antibodies were purchased from BioLegend. For Th1 polarization, WT naïve CD4+ cells were activated for 3 days in the presence of 10 ng/ml IL-12 (PeproTech) and 5 μg/ml anti-IL-4. Cell cultures were treated with DMSO or 100 nM trametinih. IFN-γ and IL-4 expressions were assessed by flow cytometry analysis. For iTreg polarization, FoxP3GFP− naïve CD4+ T cells were activated for 3 days in the presence of 3 ng/ml TGF-β1. Cell cultures were treated with DMSO or 100 nM trametinih. FoxP3GFP expression was assessed by flow cytometry analysis.

In vitro suppression assay. The responder CD4+ T cells were isolated from CD45.1+ congenic mice by using the DYNABEADS™ UNTOUCHED™ Mouse CD4 Cells Kit (Thermo Fisher Scientific), and then labeled with CTV. DYNABEADS™ FLOWCOMP™ Mouse CD4+CD25+ Treg Cells Kit (Thermo Fisher Scientific) was used to isolate Treg cells from the spleens of naïve B6 or Irf4^{fl/fl} Cd4-Cre mice or from the spleens of Irf4^{fl/fl}Cd4-Cre recipients at day 7 after heart transplantation (treated with either Rat IgG or anti-CTLA-4 plus anti-PD-L1 mAbs). CTV-labeled CD45.1+CD4+ T cells were added at about 1×10⁵ cells/well in 96-well round bottom tissue-culture plates together with or without equal numbers of Treg cells from different groups, and stimulated with T-cell-depleted mitomycin C-treated B6 splenocytes and 1 μg/ml soluble anti-CD3e mAh. Three days later, CD45.1+CD4+ T cells were analyzed for proliferation by CTV dilution using a LSR II flow cytometer.

Quantitative RT-PCR. Total RNA was extracted from activated WT and Irf4^{-/-} CD4+ T cells using a RNeasy mini kit (Qiagen), and cDNA was synthesized using the iSCRIPT™ Reverse Transcription Supermix (Bio-rad). All target primers were predesigned KiCqStart Primers (Sigma-aldrich). Transcription of target genes was calculated according to the 2-ΔCT method, as described by the manufacturer (CFX96 Touch Real-Time PCR Detection System; Bio-rad). Gene expression results were expressed as arbitrary units relative to the expression of Gapdh.

Immunoblot analysis. Protein extracts were resolved by SDS-PAGE, transferred onto an Immunobilon membrane, and analyzed by immunoblot with anti-IRF4 (sc-6059; Santa Cruz), anti-BATF (8638; Cell Signaling Technology), and anti-β-actin (12262; Cell Signaling Technology). Horseradish peroxidase-linked antibody to rabbit immunoglobulin G (7074; Cell Signaling Technology), horseradish peroxidase-linked antibody to goat immunoglobulin G (sc-2768; Santa Cruz), and horseradish peroxidase-linked antibody to mouse immunoglobulin G (7076; Cell Signaling Technology) were used as secondary antibodies. Protein expression was detected by chemiluminescence.

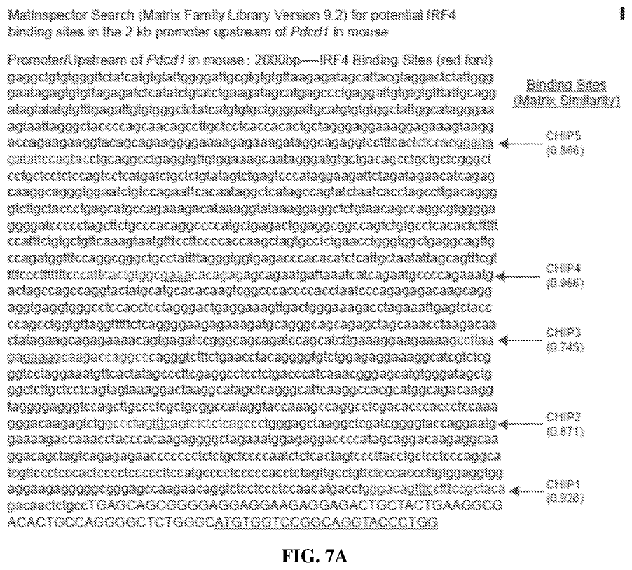

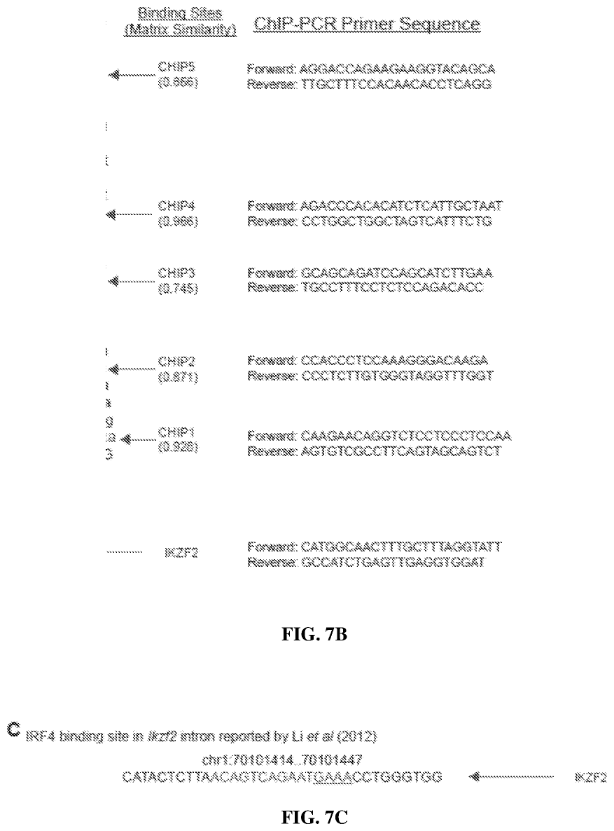

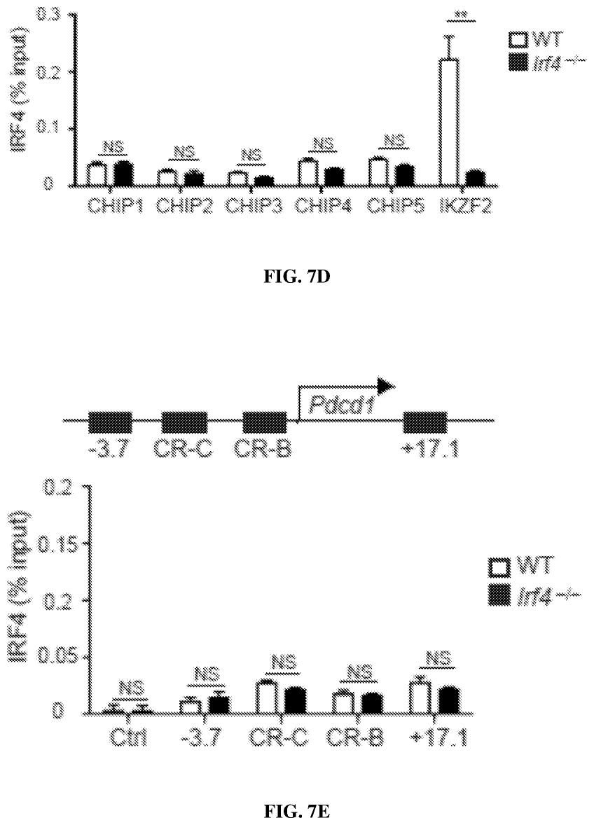

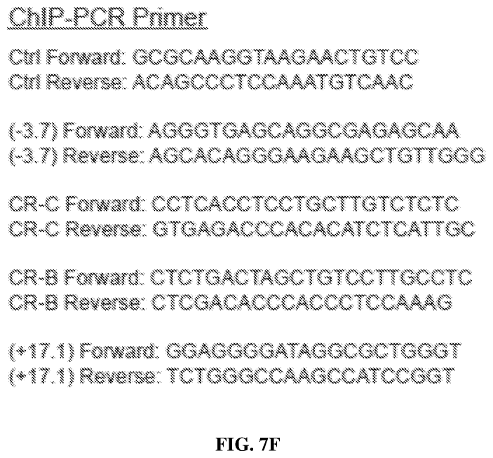

Chromatin immunoprecipitation. Chromatin immunoprecipitation (ChIP) assays were performed as previously described (Xiao et al., 2016). All primers used in ChIP-PCR and the reported IRF4 binding site in Ikzf2 intron (Li et al., 2012) are displayed in FIGS. 7B and 7C. In brief, naïve WT or Irf4^{-/-} CD4+ T cells were activated in vitro for 48 hours and then fixed with formaldehyde. Chromatin was extracted from 1×10⁶ cells for each immunoprecipitation. Anti-Histone H3K9me3 (61013; Active Motif), anti-H3K4me3 (17-678; Millipore), anti-H3Ac (39139; Active Motif), anti-H4Ac (39925; Active Motif), anti-Helios (sc-9864; Santa Cruz), anti-IRF4 (sc-6059, Santa Cruz), and isotype-matched control antibody (sc-2027; Santa Cruz) were used for immunoprecipitation of chromatin with an EZ ChIP kit according to the manufacturer's instructions (Millipore). The precipitated DNA was then analyzed by real-time PCR. Data are presented as relative binding based on normalization to input DNA.

Retrovirus-mediated gene expression. cDNA fragments encoding mouse Irf4 and Ikzf2 were amplified by PCR and then cloned into a pMYs-IRES-EGFP retroviral vector (Cell Biolabs). Retroviral particles were produced by transfecting plat-E cells with those retroviral vectors according to the manufacturer's recommendations (Cell Biolabs). For T cell transduction, naïve WT or Irf4^{-/-} CD4+ T cells were activated for 24 hours with B6 APCs and 1 μg/ml soluble anti-CD3 mAb, and then incubated with freshly prepared retroviral particles by centrifugation for 2 hours at 780 g and 32° C. in the presence of 8 μg/ml polybrene (Sigma-Aldrich). After centrifugation, cells were first cultured for 6 hours at 32° C., and subsequently cultured for additional 3 days in complete RPMI 1640 medium at 37° C. prior to flow cytometry analysis. For preparing IRF4 re-introduced alloreactive Irf4^{-/-} CD4+ T cells, Balb/c splenic DCs were isolated by using the Pan Dendritic Cell Isolation Kit (Miltenyi Biotec). Irf4^{-/-} CD4+ T cells were activated for 3 days with Balb/c splenic DCs and 100 IU IL-2, and incubated with freshly prepared retroviral particles as mentioned above. Cells were cultured for one day after transduction, and then adoptively transferred into Irf4^{fl/fl}Cd4-Cre mice on day 1 post-heart transplantation.

Retrovirus-mediated Ikzf2 knockdown. The shRNA sequences for Ikzf2 were designed by using a publicly available online tool (https://rnaidesigner.thermofisher.com). Five top shRNA sequences listed were selected and synthesized at IDT. Two complementary oligos were annealed at annealing buffer and ligated into shRNA vector (pSIREN-RetroQ-ZsGreen) from Clontech. After Bam HI and EcoRI digestion, positive colonies were further verified by direct sequencing. The knockdown efficiency for each shRNA was measured in activated Irf4^{-/-} CD4+ T cells by FACS, and the most potent shRNAs (Target sequence: GCAGGTCATGAGTCACCATGT) were selected for performing the following experiment: Irf4^{-/-} CD4+ T cells were activated for 24 hours with B6 APCs and 1 μg/ml soluble anti-CD3 mAb, and then incubated with freshly prepared retroviral particles by centrifugation for 2 hours at 780 g and 32° C. in the presence of 8 μg/ml polybrene. After centrifugation, cells were first cultured for 6 hours at 32° C., and subsequently cultured for additional 3 days in complete RPMI 1640 medium at 37° C. prior to assessing Helios and PD-1 expressions.

Microarray and GO Enrichment Analysis. Microarray was performed by the Genomic and RNA Profiling Core at Baylor College of Medicine and data generated has been deposited in NCBI's Gene Expression Omnibus with accession number GSE83283. Naïve CD4+ T cells (CD62L+CD44−) were sorted from WT B6 or Irf4^{-/-} mice by a FACS Aria flow cytometer, and activated in vitro with soluble anti-CD3 mAh (2C11) and mitomycin C-treated APCs in 96-well round bottom tissue-culture plates. Forty-eight hours later, living CD4+ T cells were sorted from cultures by flow cytometry. Total RNA was extracted with RNeasy mini kit (Qiagen). RNA was quantified using a NanoDrop-1000 spectrophotometer and quality was monitored with the Agilent 2100 Bioanalyzer (Agilent Technologies). Cyanine-3 labeled cRNA was prepared from 0.5 μg RNA using the One-Color Low RNA Input Linear Amplification PLUS kit (Agilent Technologies) and hybridized to Agilent SurePrint G3 Mouse GE v2 8x60K Microarray (G4852B; 074809). Slides were scanned immediately after washing on the Agilent Technologies Scanner (G2505C). Data were normalized and analyzed using the Subio Platform (Subio). Gene Ontology (GO) enrichment was performed using PANTHER (Annotation Version: GO Ontology database Released 2015 Aug. 6).

Quantification and statistical analysis. Data were represented as mean±SD and analyzed with Prism version 7.0a (GraphPad Software). The P values of EAE clinical scores and graft survival were determined by the Mann-Whitney test. Other measurements were performed using unpaired Student's t-test. The sample size chosen for the animal experiments was estimated based on the literature and our previous experience of performing similar sets of experiments. Differences were considered significant when P<0.05. P values are denoted in figures as follows: n.s.: P>0.05; *: P<0.05; and **: P<0.01.

Data and software availability. Microarray data for this project have been deposited at NCBI's Gene Expression Omnibus (GEO) with the accession number GSE83283.

Results

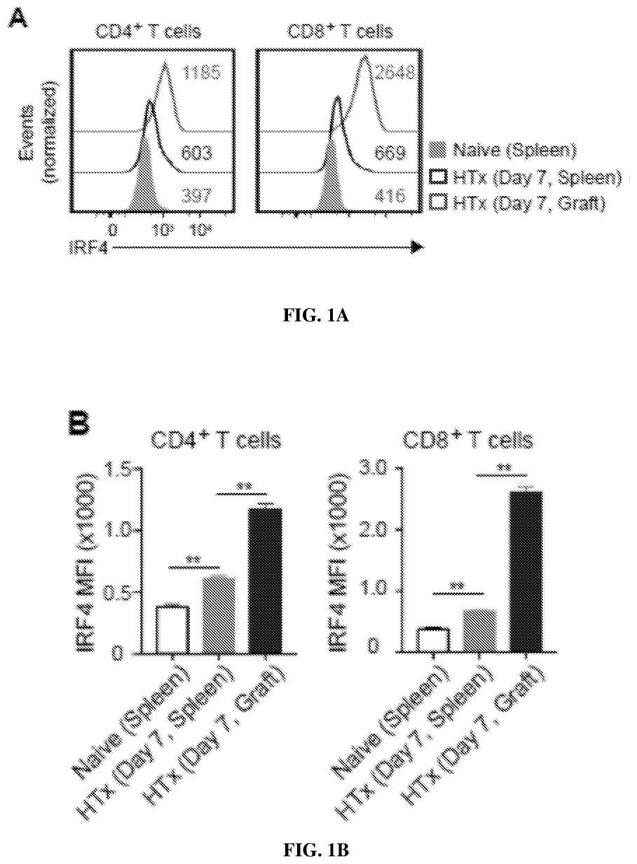

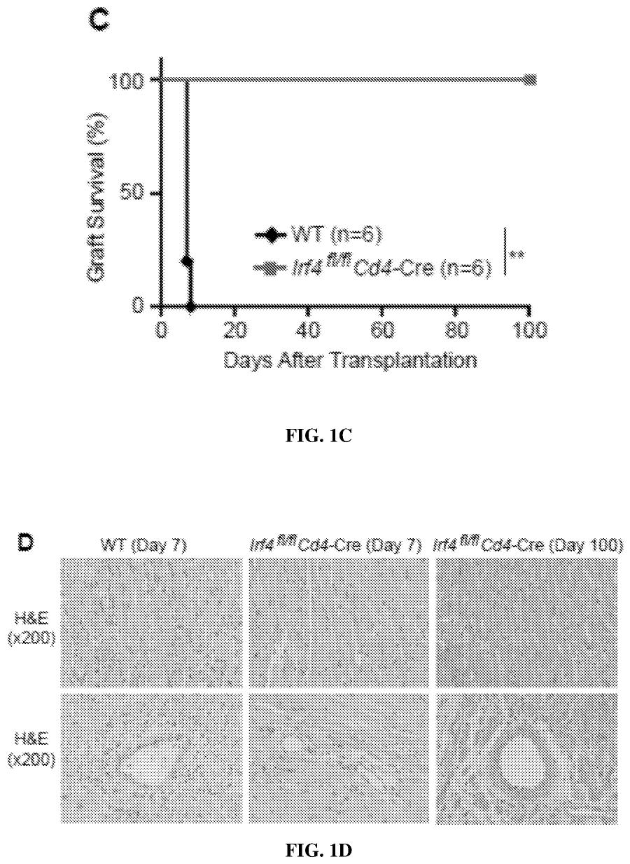

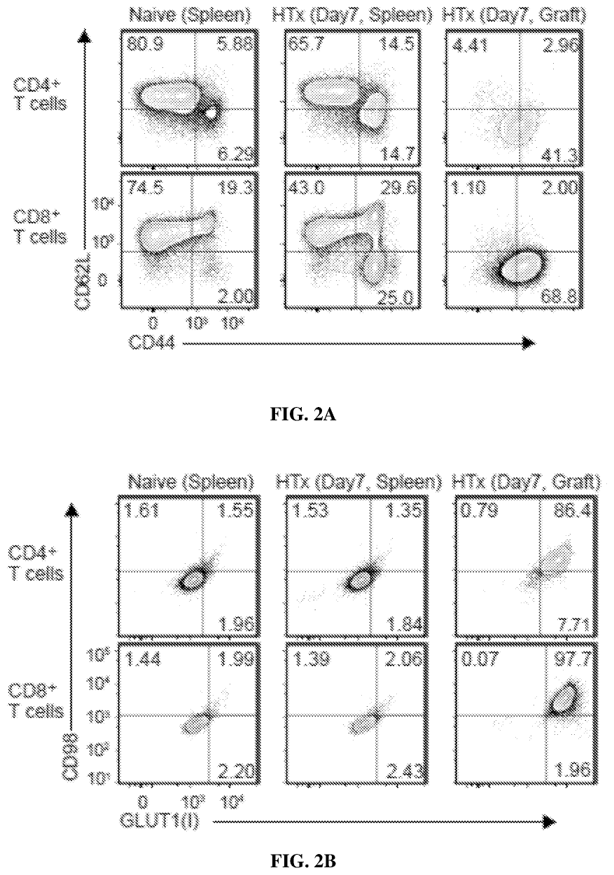

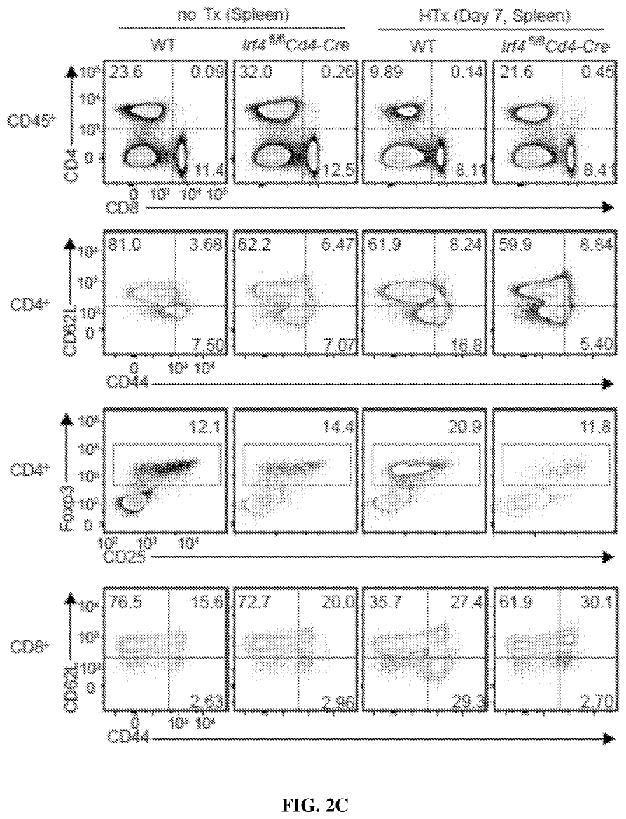

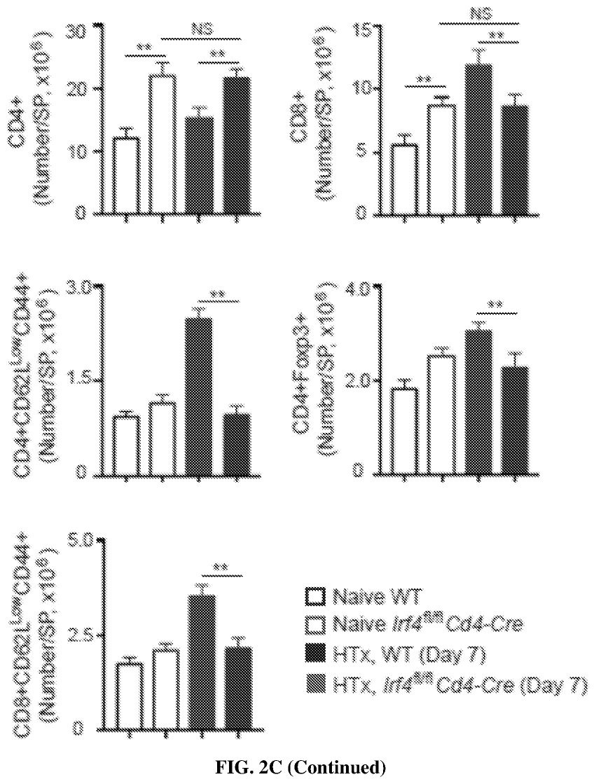

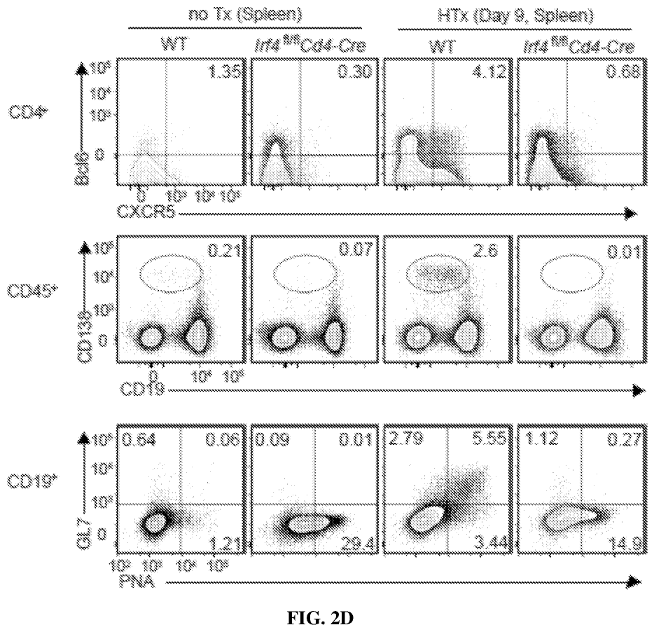

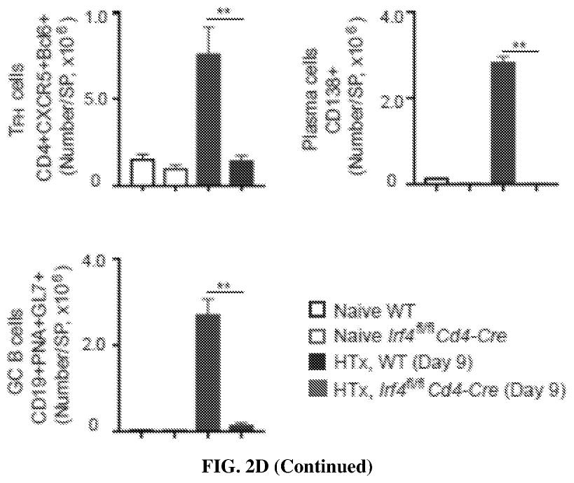

IRF4 is induced in graft-infiltrating T cells and is required for heart transplant rejection. IRF4 is a key transcription factor for translating TCR signaling into proper T cell responses (Huber and Lohoff, 2014), but its role in T cell-mediated transplant rejection remains unclear. Here, IRF4 expression in T cells in response to heart transplantation was first assessed. Balb/c hearts were transplanted (abbreviated as “HTx”) into fully MHC-mismatched C57BL/6 (B6) recipients. Splenocytes and graft-infiltrating cells were harvested and analyzed at 7 days post-transplant when heart allografts were rejected. Graft-infiltrating T cells expressed a significantly high mean fluorescence intensity (MFI) of IRF4; whilst splenic T cells from transplanted mice had a moderate increase in IRF4 expression compared to that of naïve B6 mice (FIGS. 1A and 1B). The majority of graft-infiltrating T cells had lost CD62L expression, but expressed T cell activation markers CD44, glucose transporter 1 (GLUT1), and CD98 (FIGS. 2A and 2B). Thus, IRF4 was expressed in graft-infiltrating T cells and was correlated to the activation status of these cells.

To investigate whether IRF4 expression in T cells plays a role in transplant rejection, Balb/c hearts were transplanted into T cell-specific IRF4 knockout (Irf4^{fl/fl}Cd4-Cre; B6 background) or wild-type (WT) B6 mice. None of the Irf4^{fl/fl}Cd4-Cre mice rejected their Balb/c heart allografts (median survival time (MST) of >100 days; n=6), whereas WT B6 mice rejected Balb/c hearts acutely (MST=7.17±0.41 days; n=6) (FIG. 1C). Histology of heart allografts harvested from Irf4^{fl/fl}Cd4-Cre recipient mice at days 7 and 100 post-transplant showed intact myocytes with minimal cellular infiltration and vasculopathy (FIG. 1D). Hence, selective ablation of IRF4 in T cells reduced their ability to reject heart allografts, which provides a means for achieving graft acceptance.

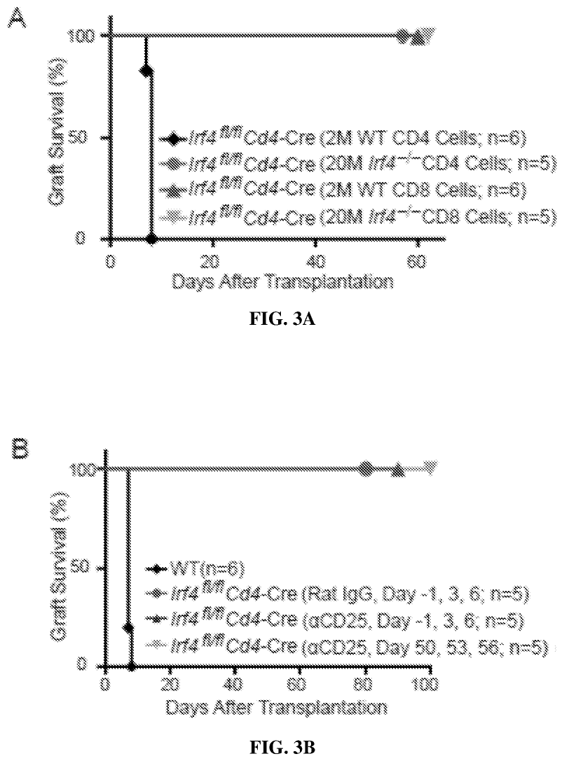

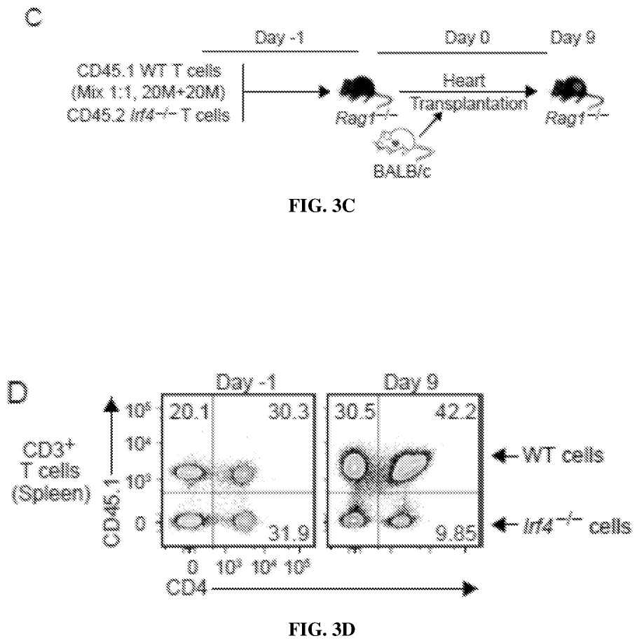

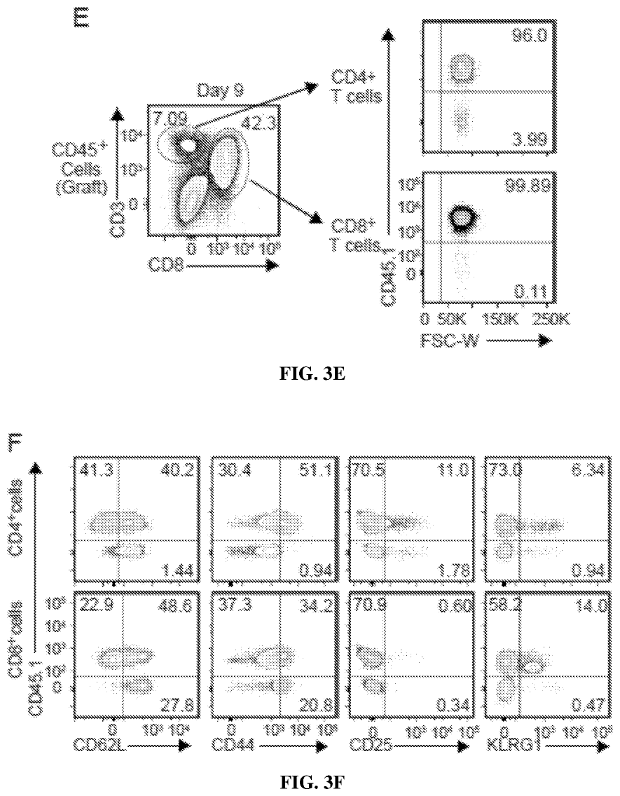

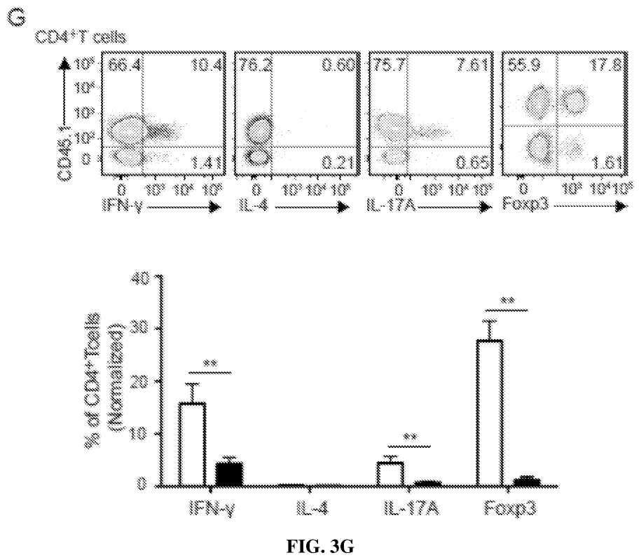

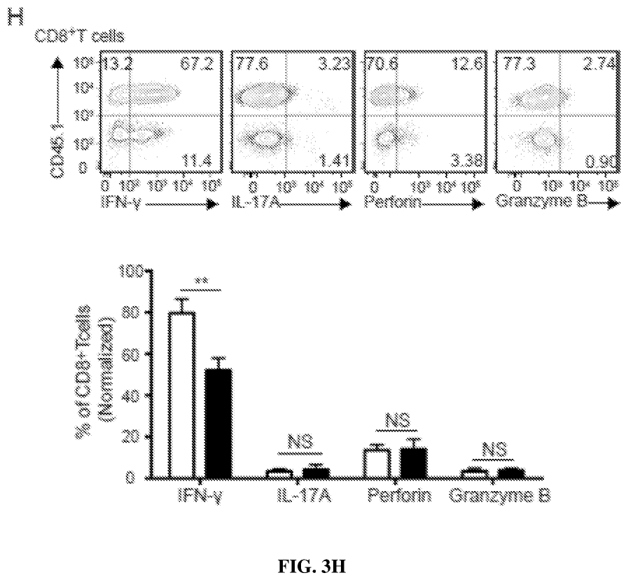

IRF4 is important in T cell differentiation and accumulation of T cells in heart allografts. To determine whether a lack of functional T cells in Irf4^{fl/fl}Cd4-Cre mice accounts for graft acceptance, Irf4^{fl/fl}Cd4-Cre mice were adoptively transferred with 2 million WT B6 CD4+ or CD8+ T cells, or 20 million Irf4^{-/-} CD4+ or CD8+ T cells one day prior to Balb/c heart transplantations. Irf4^{fl/fl}Cd4-Cre recipients transferred with 2 million WT B6 CD4+ T cells acutely rejected their Balb/c heart allografts (MST=7.83±0.41 days), whereas none of the Irf4^{fl/fl}Cd4-Cre recipients in other groups rejected the heart allografts (FIG. 3A). These results showed that the lack of functional CD4+(but not CD8+) T cells resulted in heart allograft acceptance, and that increasing the number of dysfunctional Irf4-deficient CD4+ T cells failed to restore transplant rejection.

CD4+CD25+Foxp3+ Treg cells are essential to maintain long-term allograft survival in many experimental models (Miyahara et al., 2012). To determine whether Treg cells contribute to the allograft acceptance in Irf4^{fl/fl}Cd4-Cre mice, Balb/c hearts were transplanted into Irf4^{fl/fl}Cd4-Cre mice and treated with PC61 anti-CD25 mAh either on days −1,3, and 6 (induction phase of graft acceptance) or on days 50, 53, and 56 (maintenance phase), or treated with a control IgG on days −1, 3, and 6 post-transplantation. Injection of PC61 mAh reduced CD4+FoxP3+ cells by approximately 70% in peripheral blood of recipient mice one day after treatment completed. Nevertheless, this partial Treg-cell depletion during the induction or maintenance phase did not abrogate permanent allograft survival in Irf4^{fl/fl}Cd4-Cre mice, which was essentially the same as that in control IgG group (MST of >100 days; n=5 each group) (FIG. 3B).

Intrinsic changes of Irf4-deficient T cells responsible for long-term allograft acceptance were then identified and analyzed. Balb/c hearts were transplanted into Irf4^{fl/fl}Cd4-Cre or WT B6 mice. Before transplantation, Irf4^{fl/fl} Cd4-Cre mice had significantly more T cells in spleens than did WT B6 mice. At day 7 post-transplant, T cell numbers in the spleens of Irf4^{fl/fl}Cd4-Cre mice remained largely unchanged (similar to that in un-transplanted Irf4^{fl/fl}Cd4-Cre mice), while the number of splenic T cells (particularly CD8+ T cells) in WT recipients increased (FIG. 2C). These results showed that the expansion of alloreactive T cells in Irf4^{fl/fl}Cd4-Cre recipients was impaired. Moreover, frequencies of CD4+CD62L^{low}CD44+, CD8+CD62L^{low}CD44+, and CD4+Foxp3+ splenocytes from Irf4^{fl/fl}Cd4-Cre recipients were significantly lower than those of WT recipients (FIG. 2C). CD4+ BCL6+CXCR5+ Tfh cells, CD19^{low}CD138+ plasma cells, and CD19+GL7+PNA+ germinal center B cells were absent in the spleens of Irf4^{fl/fl}Cd4-Cre recipients, but were clearly detected in WT recipients at day 9 post-transplant (FIG. 2D). Hence, IRF4 facilitates induction of Tfh cell response to heart transplant.

An adoptive co-transfer model was used to further assess the intrinsic changes of Irf4-deficient T cells. CD45.1+WT T cells and CD45.2+ Irf4^{-/-} T cells were co-injected at a 1:1 ratio into B6.Rag1^{-/-} mice one day before receiving Balb/c heart allografts (FIG. 3C). The heart grafts were rejected between 9 to 15 days post-transplant. At day 9 post-transplant, about 30% of transferred cells in spleens were Irf4^{-/-} T cells (FIG. 3D). A large majority of graft-infiltrating T cells were CD45.1+WT T cells (FIG. 3E), demonstrating that Irf4^{-/-} T cells lost the ability to infiltrate allografts. The infiltrating Irf4^{-/-} T cells were too few in number to be compared with WT T cells, and thus the co-injected T cells in the spleens were compared. Both CD4+ and CD8+ Irf4^{-/-} T cells did not down-regulate CD62L and barely expressed an effector marker KLRG1. CD4+ Irf4^{-/-} T cells also failed to upregulate CD44 expression (FIG. 3F). In addition, the frequencies of IFN-γ- and IL-17-producing cells within CD4+ Irf4^{-/-} T cells were significantly lower than those of CD4+WT T cells (FIG. 3G), and the frequency of IFN-γ-producing cells within CD8+ Irf4^{-/-} T cells was also lower than that of CD8+WT T cells (FIG. 3H). Thus, IRF4 deficiency inhibited the expression of effector T cell markers and the production of signature cytokines for effector T cells in response to heart transplantation. The frequency of Foxp3+ cells within CD4+ Irf4^{-/-} splenic T cells was lower than that of CD4+WT splenocytes (FIG. 3G).

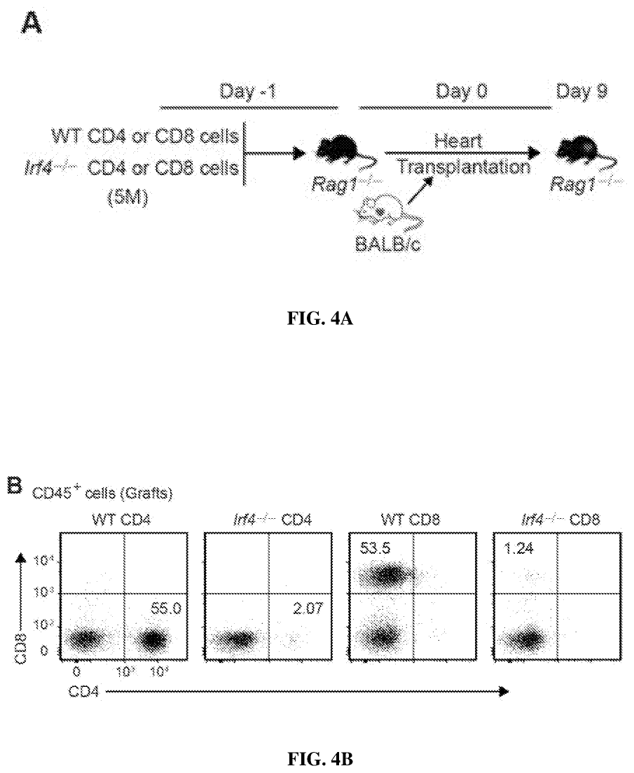

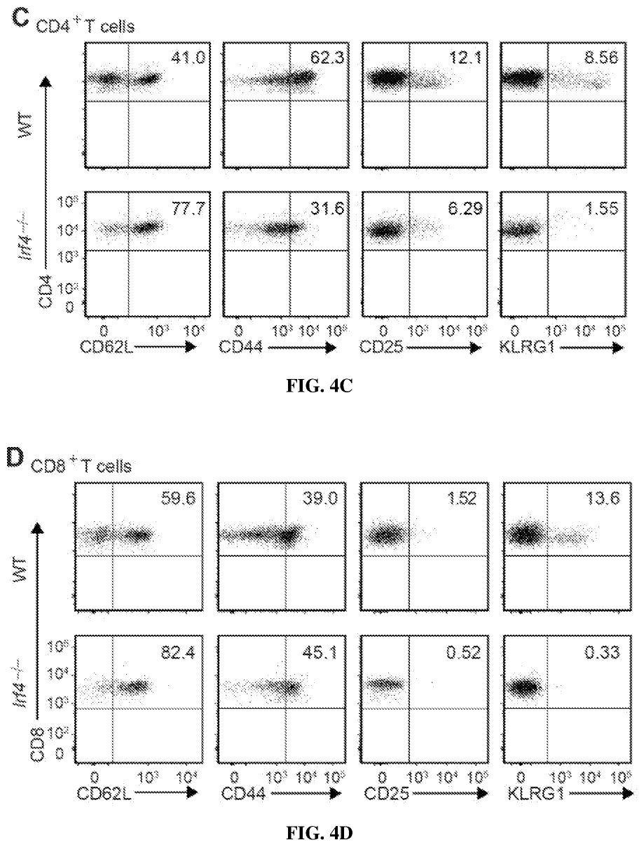

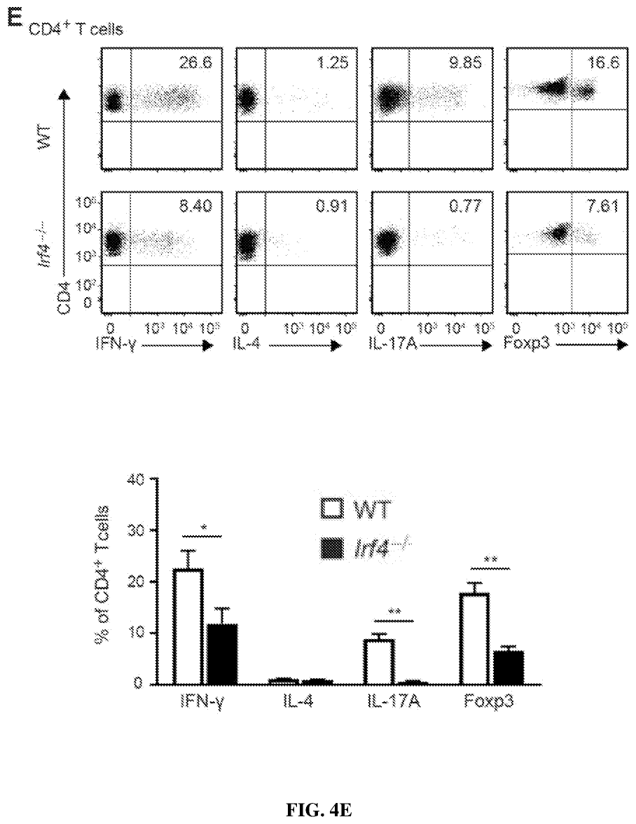

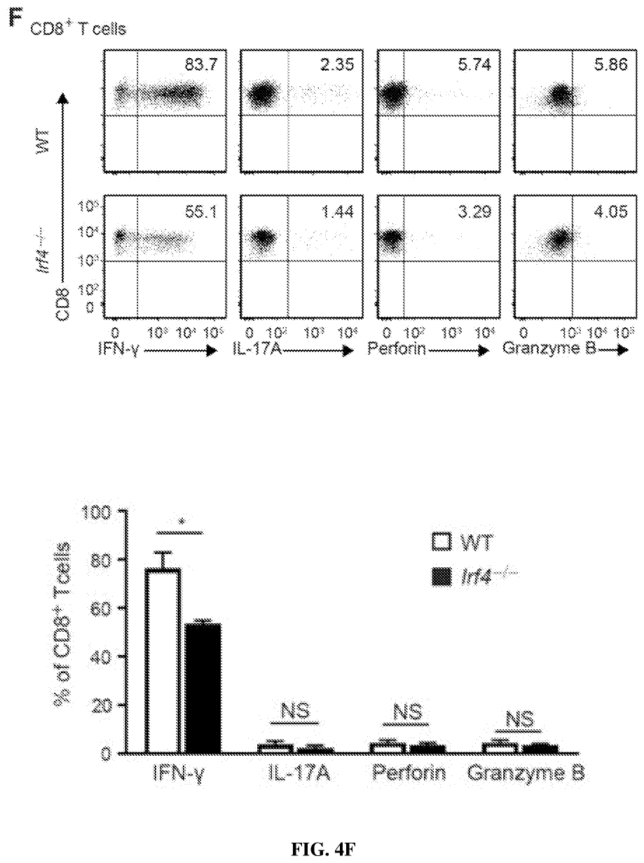

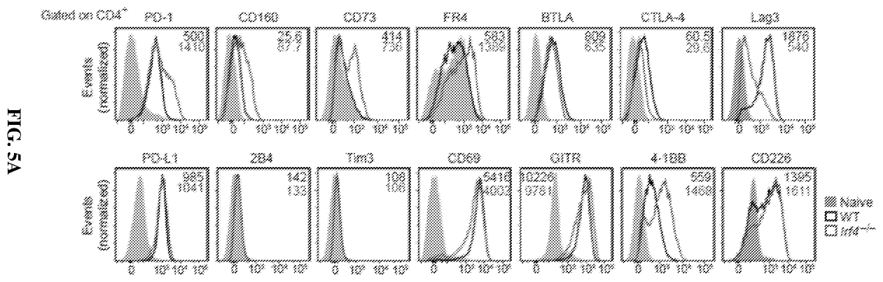

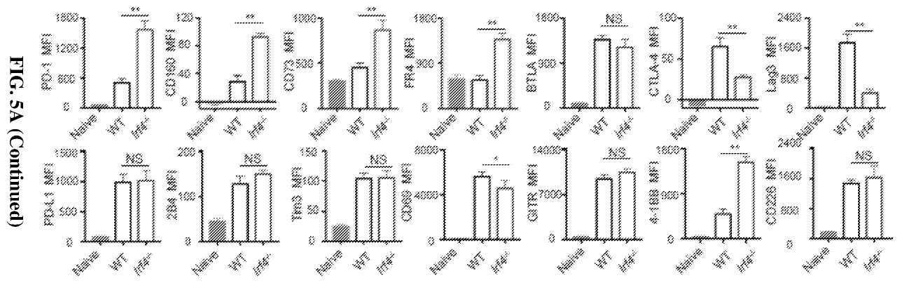

Next, these experiments were repeated by separately transferring sorted CD4+WT, CD4+ Irf4^{-/-}, CD8+WT, or CD8+ Irf4^{-/-} T cells into B6.Rag1^{-/-} mice one day prior to BALB/c heart transplantation (FIG. 4A). Ex vivo analysis of transferred cells was performed on day 9 post-transplant. Consistent with results from the co-transfer model, separately injected CD4+ and CD8+ Irf4^{-/-} T cells also lost their ability to infiltrate allografts (FIG. 4B). Compared to the separately transferred WT T cells in spleens, CD4+ and CD8+ Irf4^{-/-} T cells largely maintained CD62L expression, barely expressed KLRG1, and produced significantly less IFN-γ (FIGS. 4C-4F). CD4+ Irf4^{-/-} T cells also produced significantly less IL-17 and expressed less Foxp3 (FIG. 4E). Taken together, IRF4 promoted effector T cell differentiation and infiltration into heart allografts.

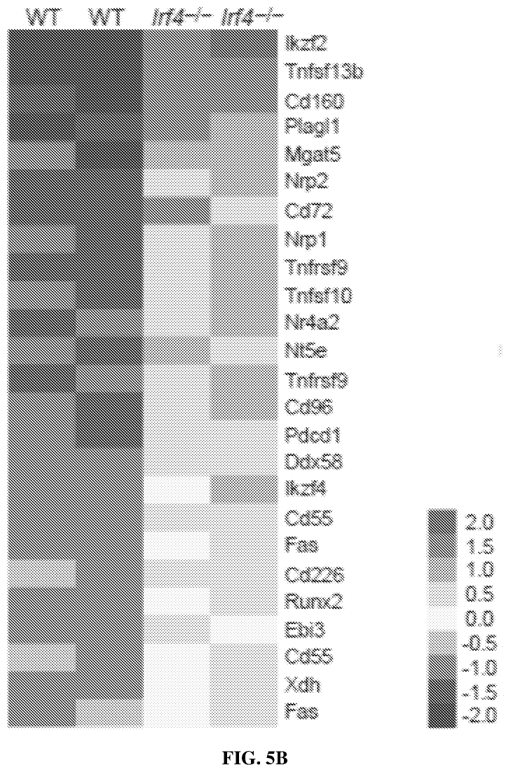

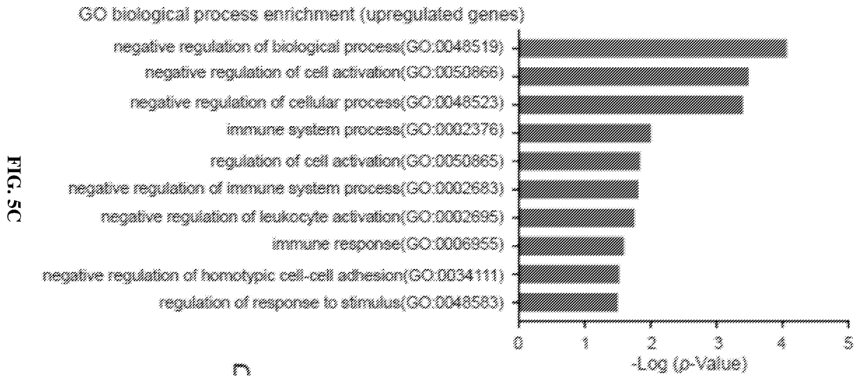

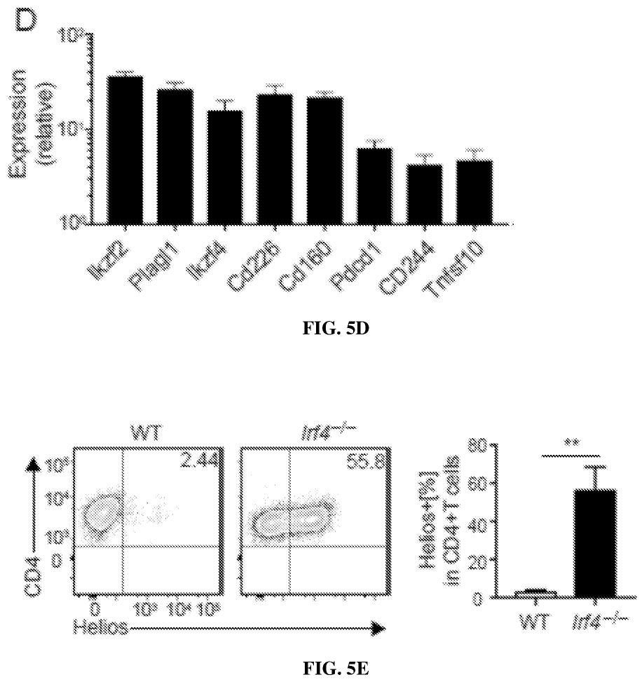

IRF4 represses a group of molecules associated with CD4+ T cell dysfunction. Lack of functional CD4+ T cells facilitated graft acceptance in Irf4^{fl/fl}Cd4-Cre mice. Next, the intrinsic mechanism underlying the dysfunction of Irf4-deficient CD4+ T cells was evaluated. Expression of inhibitory and costimulatory receptors on Irf4^{-/-} or WT CD4+ T cells one day after in vitro activation was measured. Compared to WT CD4+ T cells, Irf4^{-/-} CD4+ T cells expressed higher MFIs of exhaustion and anergy signatures including PD-1, CD160, CD73, and folate receptor 4 (FR4) (Martinez et al., 2012; Wherry and Kurachi, 2015), and also expressed a similar or slightly lower MFIs of BTLA and CTLA-4. Profoundly, another essential exhaustion marker, LAG-3, was significantly decreased on Irf4^{-/-} CD4+ T cells (FIG. 5A).

Microarray analysis was used to compare gene expression profiles between Irf4^{-/-} and WT CD4+ T cells following two-day in vitro activation. Among 672 differentially expressed genes, 438 were increased in activated Irf4^{-/-} T cells, and were significantly enriched in the Gene Ontology (GO) categories of “negative regulation of biological process” and “negative regulation of cell activation” (FIG. 5C). Ikzf2 (encoding Helios), Pdcd1 (encoding PD-1) and Cd160 were among the highest upregulated genes in activated Irf4^{-/-} CD4+ T cells when compared to activated WT CD4+ T cells (FIG. 5B), which were confirmed by quantitative real-time PCR (FIG. 5D). Helios is a signature protein for T cell dysfunction (Crawford et al., 2014; Singer et al., 2016). Helios protein was absent in activated WT CD4+ T cells but was expressed in more than 50% of activated Irf4^{-/-} CD4+ T cells (FIG. 5E). Collectively, IRF4 repressed a group of previously defined molecules associated with CD4+ T cell dysfunction.

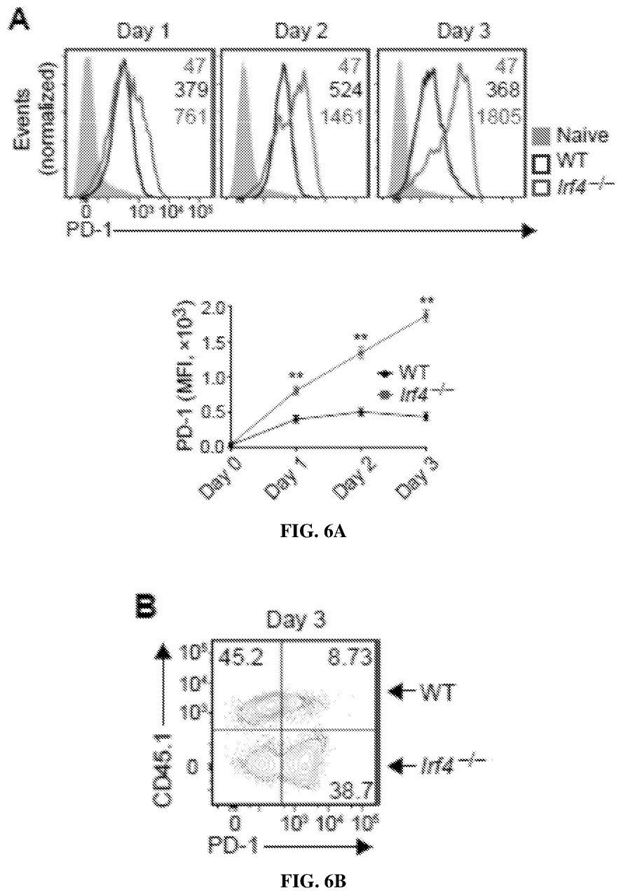

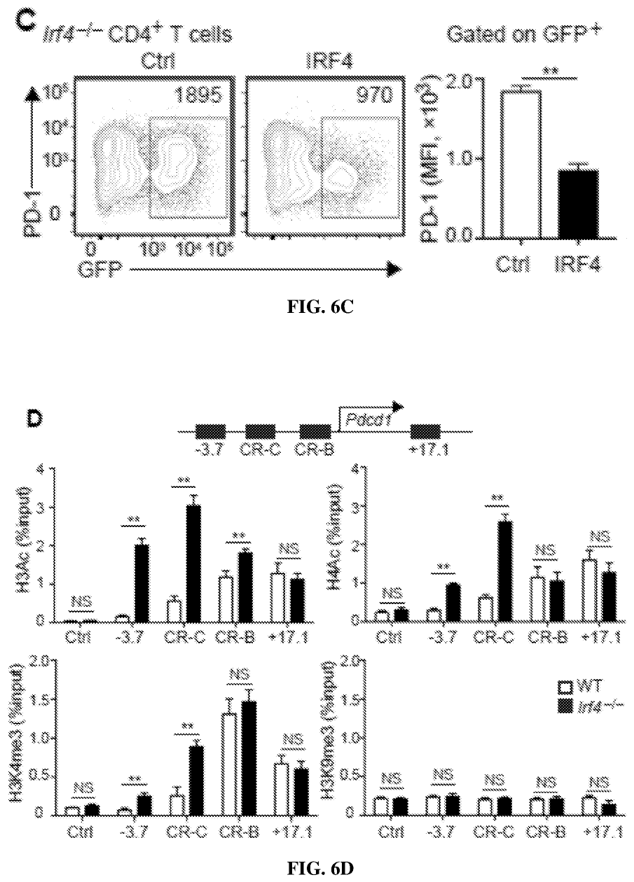

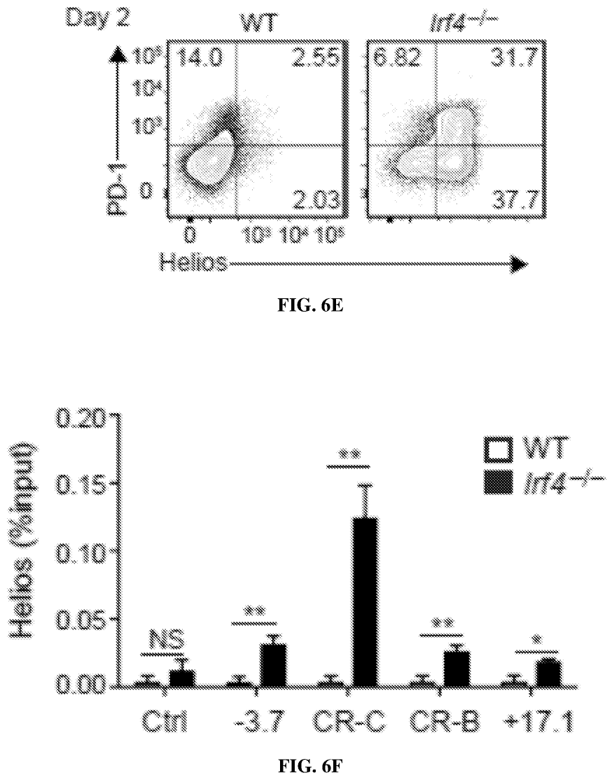

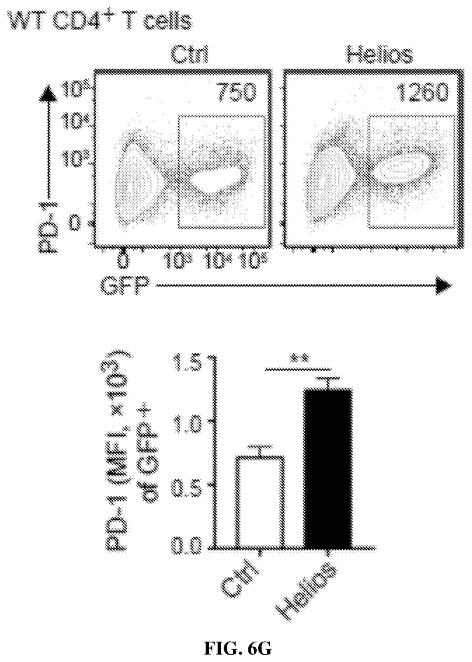

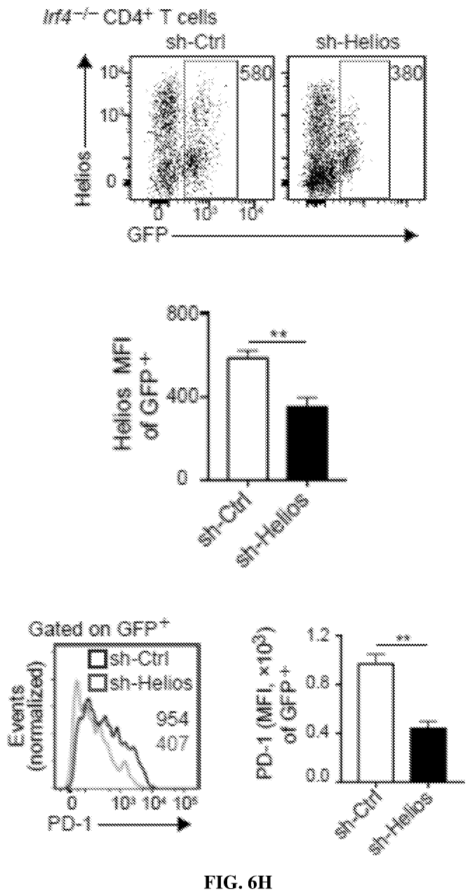

IRF4 represses CD4+ T cell expression of PD-1. Given that Pdcd1 was among the most upregulated genes Irf4^{-/-} in versus WT CD4+ T cells after activation, regulation of PD-1 expression by IRF4 was evaluated. PD-1 expression on Irf4^{-/-} CD4+ T cells was progressively increased from day 0 to day 3 upon in vitro activation (FIG. 6A), and was higher than that of co-cultured CD45.1+WT CD4+ T cells (FIG. 6B). To further examine the role of IRF4 in PD-1 expression, activated Irf4^{-/-} CD4+ T cells were transduced with a retroviral vector expressing IRF4-green fluorescent protein (GFP) or a control vector expressing GFP alone. As shown in FIG. 6C, transduction of IRF4 into activated Irf4^{-/-} CD4+ T cells (detected by GFP expression) led to a marked inhibition of PD-1 expression when compared with GFP control transduction. Thus, IRF4 expression in activated T cells repressed PD-1.

To determine how IRF4 represses PD-1 expression, Chromatin Immunoprecipitation (ChIP) analysis was performed. In activated WT CD4+ T cells (expressing IRF4), there was no detection of specific enrichment of IRF4 at the putative binding sites upstream of Pdcd1 or at a set of known cis-elements of the Pdcd1 gene, including two upstream conserved regions (CR-B and CR-C) as well as two regions located −3.7 and +17.1 kb from the Pdcd1 transcription start site (Bally et al., 2016) (FIGS. 7D and 7E). These data showed that the repression effect of IRF4 on PD-1 expression was unlikely to be related to its transcriptional activity. Thus, it was evaluated whether histone modifications were involved in the regulation of PD-1 expression by IRF4. As shown in FIG. 6D, H3 acetylation (H3Ac) was significantly increased at the −3.7 site and the CR-B and CR-C regions, whereas H4 acetylation (H4Ac) and H3 lysine 4 trimethylation (H3K4me3) were markedly increased at the −3.7 site and the CR-C region in activated Irf4^{-/-} CD4+ T cells compared with those in activated WT CD4+ T cells. H3Ac, H4Ac, and H3K4me3 are all active histone marks. The expression of a repressive histone mark, H3 lysine 9 trimethylation (H3K9me3), displayed no changes at the cis-elements of Pdcd1 in Irf4^{-/-} relative to WT CD4+ T cells. Therefore, IRF4 deficiency in activated CD4+ T cells induced an active chromatin state at the cis-elements of Pdcd1, correlating to elevated PD-1 expression.

In activated Irf4^{-/-} CD4+ T cells, most PD-1^{hi} cells were Helios positive (FIG. 6E). Thus, whether Helios regulates PD-1 expression was investigated. This possibility was supported by a ChIP assay, which detected the binding of Helios to the cis-elements of Pdcd1 in activated Irf4^{-/-} CD4+ T cells (FIG. 6F). Furthermore, activated WT CD4+ T cells (with minimal Helios expression) were transduced with a retroviral vector expressing Helios-GFP or just GFP. Transduction of Helios into activated WT CD4+ T cells resulted in increased PD-1 expression when compared with GFP control transduction (FIG. 6G). Activated Irf4^{-/-} CD4+ T cells (with Helios expression) were also transduced with a retroviral vector co-expressing GFP and short hairpin RNA (shRNA) sequences for Helios (sh-Helios) or expressing GFP alone (sh-Ctrl). As indicated by GFP+ cells, transduction with sh-Helios decreased PD-1 expression in Irf4^{-/-} CD4+ T cells (FIG. 6H). Thus, Helios promoted PD-1 expression. Taken together, IRF4 deletion progressively increased PD-1 expression on activated T cells, which was associated with the increased chromatin accessibility and Helios binding to PD-1 cis-regulatory elements.

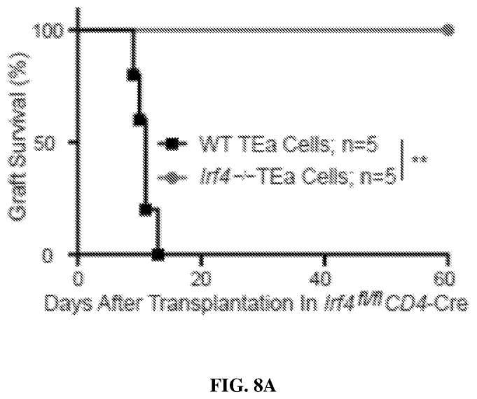

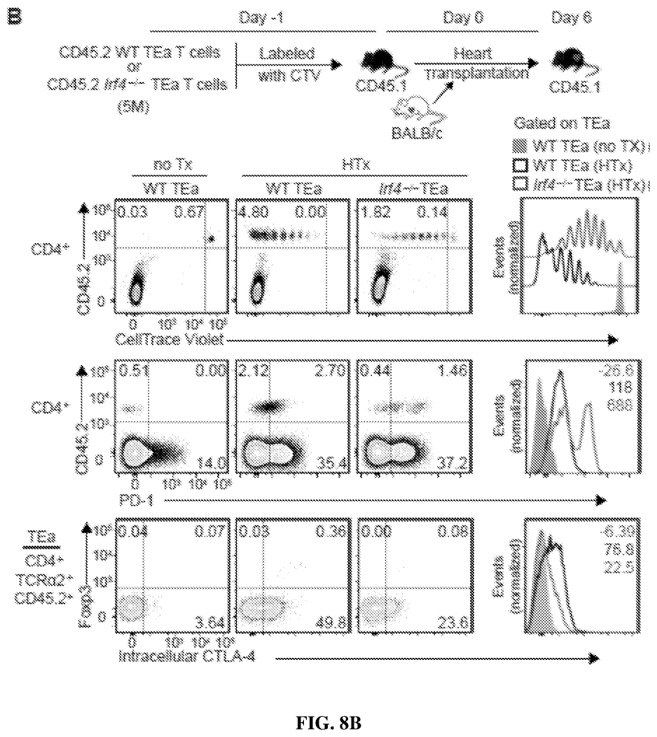

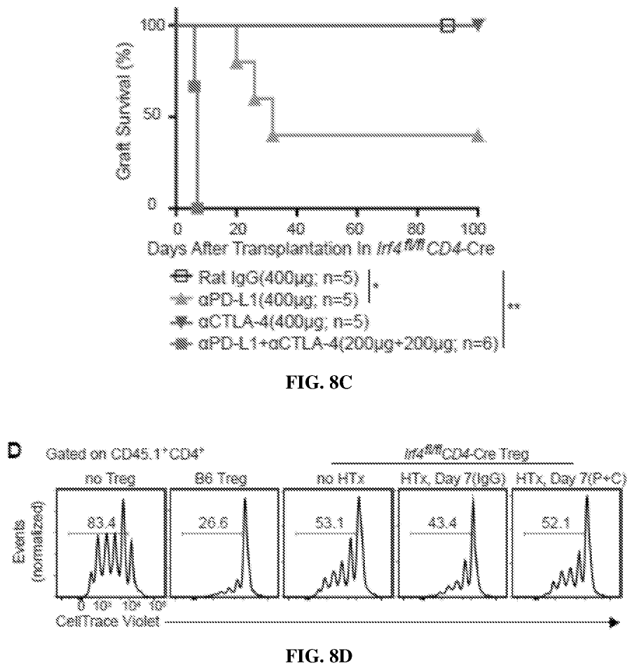

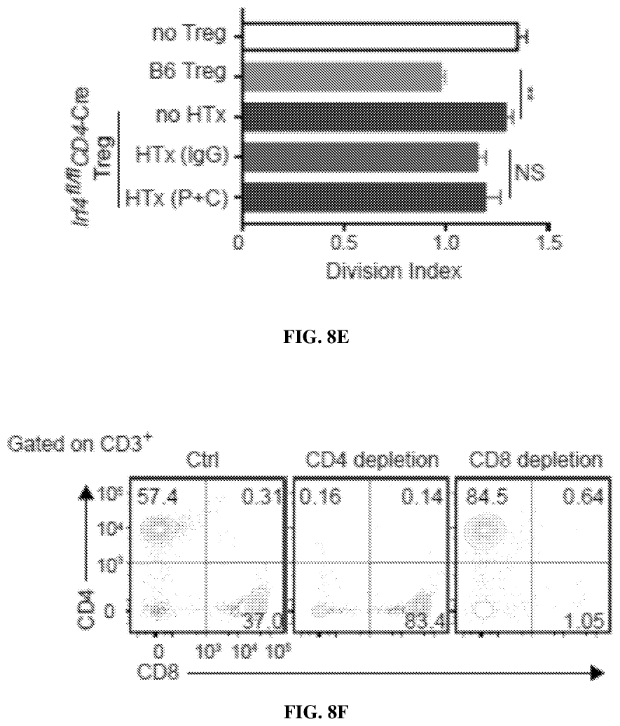

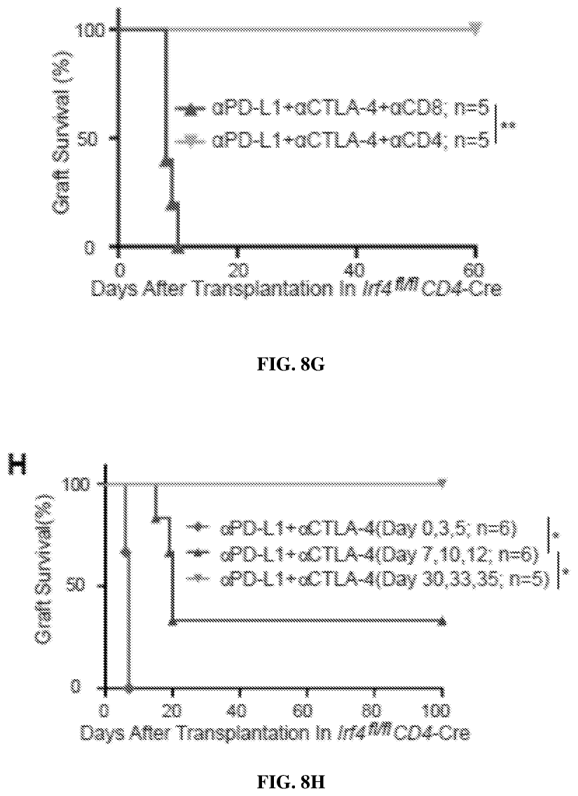

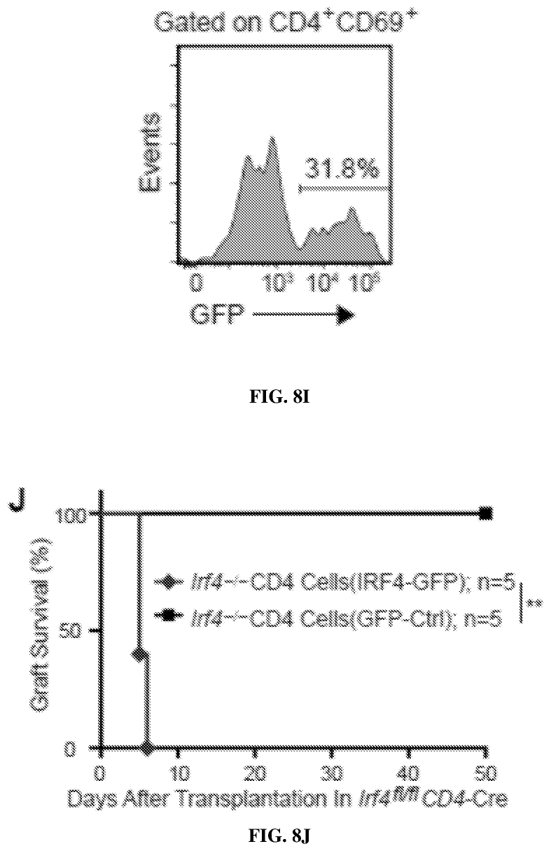

Dysfunctional state of Irf4-deficient T cells is initially reversible by PD-L1 blockade but it progressively develops into an irreversible state. TCR-transgenic TEa CD4+ T cells (B6 background) recognize a Balb/c I-Eα allopeptide presented by B6 APCs; mice containing only TEa T cells were able to reject Balb/c skin allografts (Gupta et al., 2011). Adoptive transfer of WT TEa but not Irf4^{-/-} TEa cells induced rejection of Balb/c hearts in Irf4^{fl/fl}Cd4-Cre recipients (FIG. 8A). To determine whether immune checkpoint PD-1 contributes to the dysfunction of alloantigen-specific Irf4-deficient CD4+ T cells in transplantation, PD-1 expression on Irf4^{-/-} TEa versus WT TEa cells was assessed by transferring and tracking these cells in CD45.1+B6 congenic mice. As shown in FIG. 8B, CD45.1+B6 mice were transferred with either CD45.2+WT TEa or CD45.2+ Irf4^{-/-} TEa cells on day −1, and were transplanted with Balb/c hearts or left un-transplanted on day 0. Splenocytes were analyzed on day 6 (FIG. 8B). In CD45.1+B6 mice without Balb/c heart grafting, TEa CD4+ T cells neither proliferated nor expressed PD-1. In heart transplanted mice, Irf4^{-/-} TEa cells exhibited higher PD-1 expression and a lower proliferation rate than did WT TEa cells (FIG. 8B). Thus, IRF4 deficiency promoted PD-1 expression on alloantigen-specific T cells, which was associated with decreased cell proliferation. Intracellular CTLA-4 expression was also detectable in WT and Irf4^{-/-} TEa cells in transplanted groups, and WT TEa cells expressed more intracellular CTLA-4 than that of Irf4^{-/-} TEa cells (FIG. 8B).

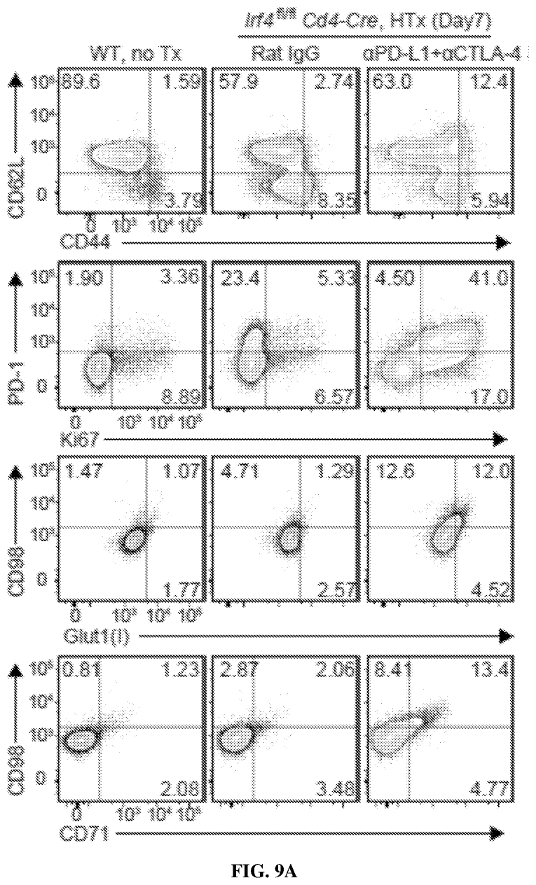

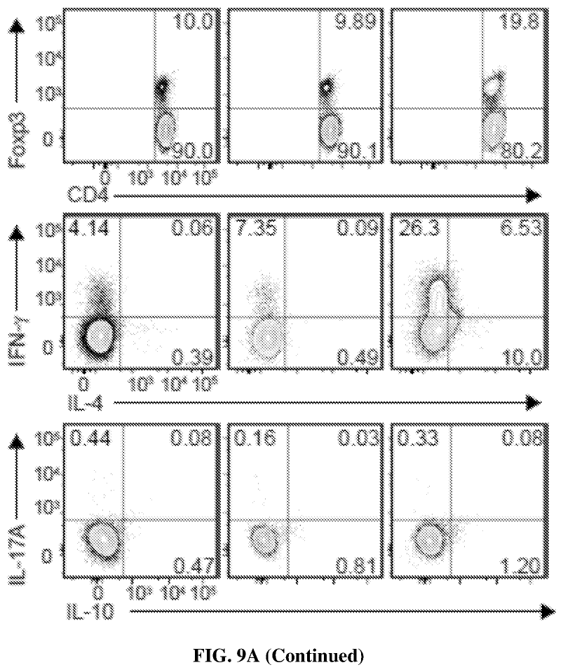

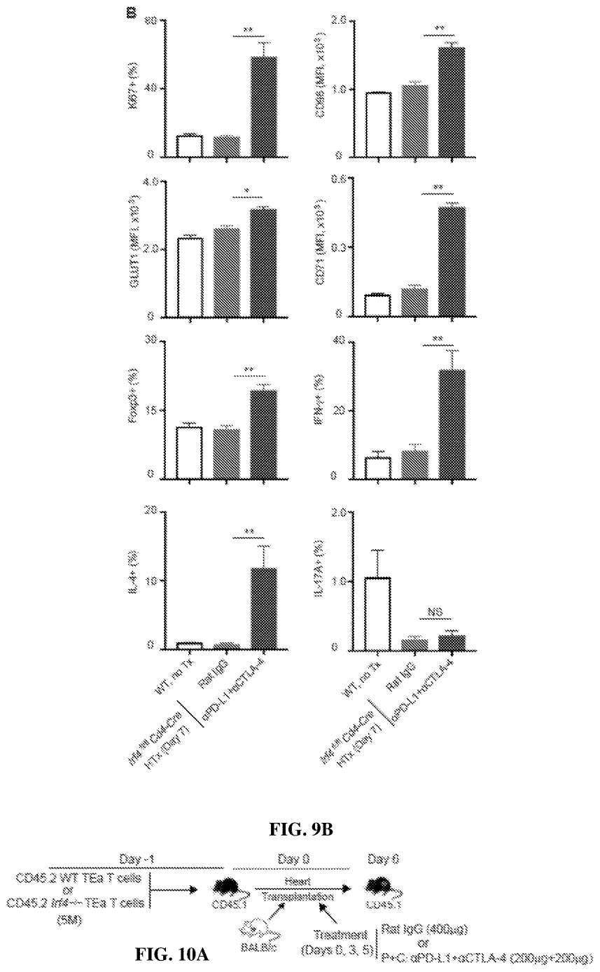

To investigate the influence of PD-1 on permanent cardiac allograft survival in Irf4^{fl/fl}Cd4-Cre recipient mice, Balb/c hearts were transplanted into Irf4^{fl/fl}Cd4-Cre mice and treated with anti-PD-L1 mAb alone, anti-CTLA-4 mAh alone, or both mAbs on days 0, 3, and 5 post-transplant. Control Irf4^{fl/fl}Cd4-Cre recipient mice were treated with rat IgG. Three of five heart allografts in anti-PD-L1 mAh-treated recipients were rejected within 35 days, whereas all allografts survived more than 100 days in recipient mice treated with anti-CTLA-4 mAh alone or rat IgG. By contrast, all six heart allografts in recipient mice treated with both anti-PD-L1 and anti-CTLA-4 mAbs were rejected on days 7-8 post-transplant (FIG. 8C). Analysis of splenocytes at day 7 post-transplant revealed that anti-PD-L1 plus anti-CTLA-4 treatment increased expression of proliferation marker Ki67, metabolic activation makers CD98 and CD71, and effector cytokine IFN-γ of Irf4-deficient CD4+ T cells (FIGS. 9A and 9B). Anti-PD-L1 plus anti-CTLA-4 treatment increased Treg cell frequency (FIGS. 9A and 9B), but those Irf4-deficient Treg cells displayed impaired suppressive function in vitro. Indeed, the suppressive function of Treg cells from all Irf4^{fl/fl}Cd4-Cre mice (including non-transplanted, or transplanted groups treated with control IgG or immune checkpoint blockade) was impaired compared to those from naïve WT B6 mice (FIGS. 8D and 8E). Depletion of CD4+ but not CD8+ T cells abrogated the capability of anti-PD-L1 plus anti-CTLA-4 treatment in restoring heart allograft rejection in Irf4^{fl/fl}Cd4-Cre recipients (FIGS. 8F and 8G). Therefore, in this checkpoint-blockade model, the reversal of CD4+(but not CD8+) T cell dysfunction was essential for restoring transplant rejection.

Balb/c hearts were also transplanted into Irf4^{fl/fl}Cd4-Cre mice and treated with both 200 μg anti-PD-L1 and 200 μg anti-CTLA-4 mAbs starting from day 0 (on days 0, 3, and 5), day 7 (on days 7, 10, and 12), or day 30 (on days 30, 33, and 35) post-transplantation. Immune checkpoint blockade starting from day 7 was not as potent as the treatment group starting from day 0, but still restored rejection of four of six heart allografts. Immune checkpoint blockade starting from day 30 post-transplant did not restore allograft rejection in Irf4^{fl/fl}Cd4-Cre mice, and all five allografts survived more than 100 days (FIG. 8H). Taken together, PD-1 was highly expressed by alloantigen-specific Irf4-deficient T cells; blockade of its ligand, PD-L1, was capable of reversing the initial dysfunctional state of Irf4-deficient T cells. Moreover, within 30 days post-transplant, Irf4-deficient T cells progressed from the reversible dysfunctional state into a “terminal” irreversible state which could not be reversed by the methods employed here.

Another approach that has been successfully applied in restoring transplant rejection in Irf4^{fl/fl}Cd4-Cre recipients was to transfer IRF4 re-introduced in Irf4^{-/-} CD4+ T cells. Irf4^{-/-} CD4+ T cells were stimulated with allogenic Balb/c splenic dendritic cells (DCs) and IL-2 for 3 days, followed by transduction with IRF4-GFP or GFP-control retrovirus for 1 day. The histogram in FIG. 8I shows the transduction efficacy of IRF4-GFP. Irf4^{fl/fl} Cd4-Cre recipients injected with one million IRF4 re-introduced (but not GFP-control transduced) Irf4^{-/-} CD4+ T cells acutely rejected heart allografts within 6 days (FIG. 8J). Therefore, IRF4 re-introduction in Irf4^{-/-} CD4+ T cells after the 3-day activation period reversed their dysfunction. This finding separates the role of IRF4 in early CD4+T cell activation from its role in subsequent dysfunctional development.

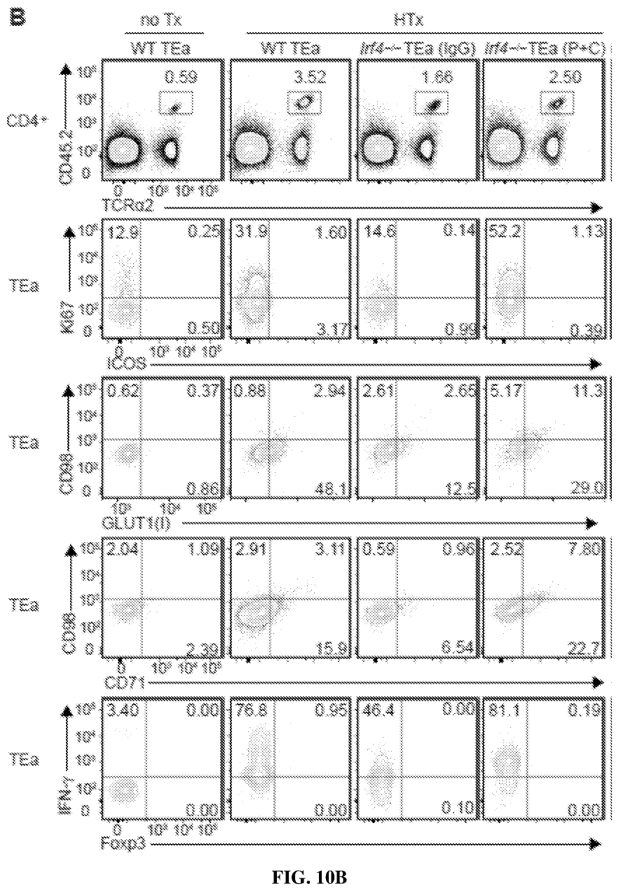

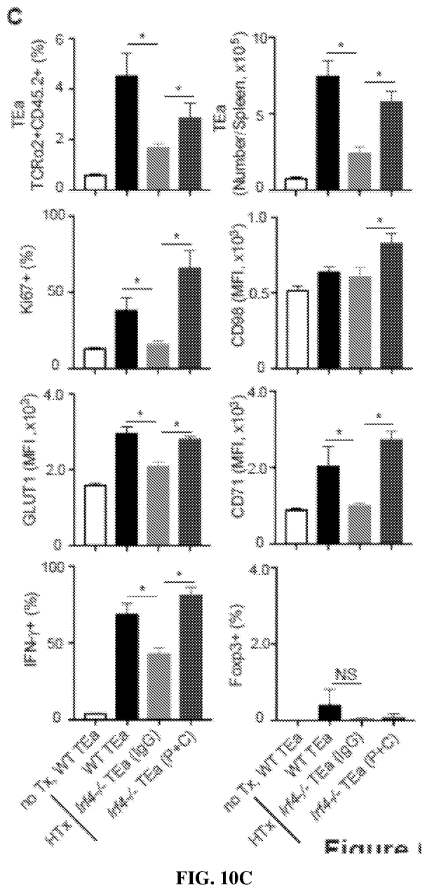

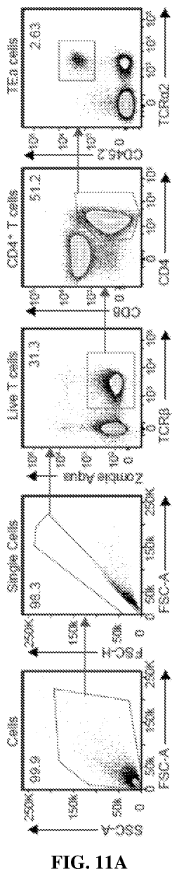

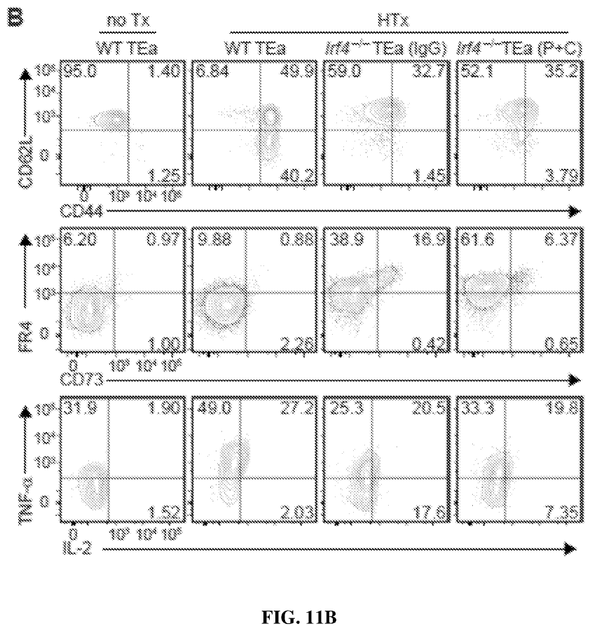

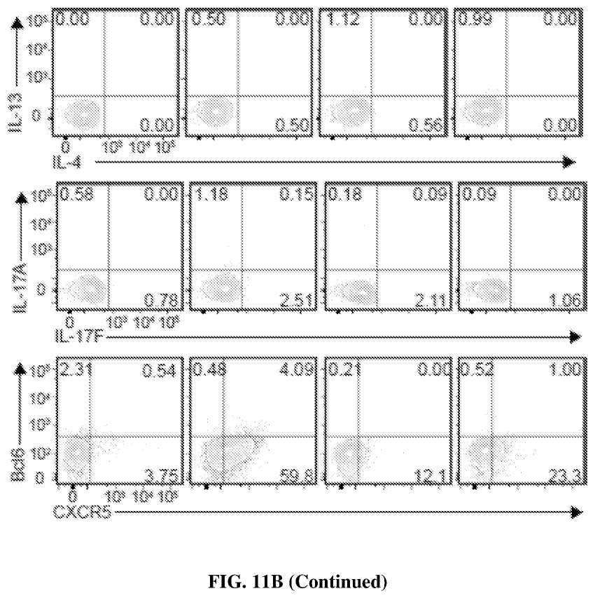

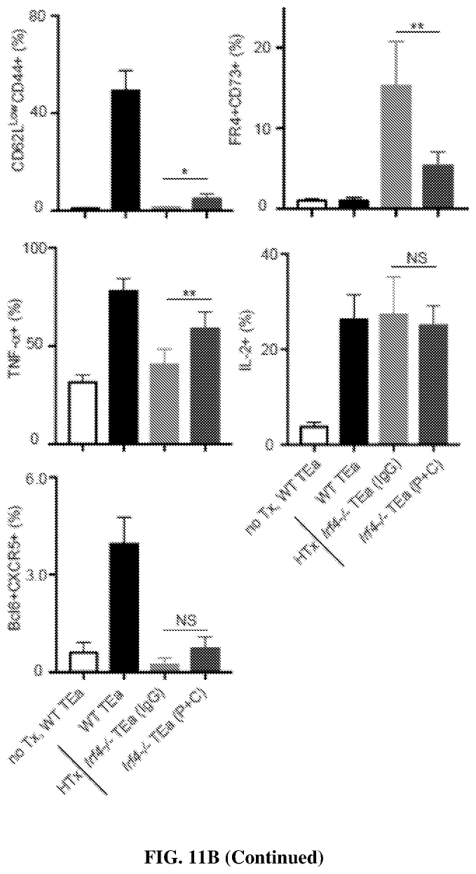

Checkpoint blockade reverses the initial dysfunction of Irf4-deficient CD4+ T cells by restoring their ability to undergo proliferation and secrete IFN-γ. The TEa cell transfer model was used to further characterize how checkpoint blockade reverses the initial dysfunction of alloantigen-specific Irf4^{-/-} CD4+ T cells. CD45.1+B6 mice were transferred with CD45.2+ Irf4^{-/-} TEa cells on day −1, transplanted with Balb/c hearts on day 0, and treated with either rat IgG (IgG group) or anti-PD-L1 plus anti-CTLA-4 mAbs (P+C group) on days 0, 3, and 5. In addition, CD45.1+B6 mice transferred with CD45.2+WT TEa cells were transplanted with Balb/c hearts or left un-transplanted. Splenocytes were analyzed on day 6 (FIGS. 10A and 11A). Checkpoint blockade affected the Irf4^{-/-} TEa cells in transplanted mice by restoring cell number, proliferation (indicated by Ki67), metabolic activation (indicated by CD98 and CD71), and IFN-γ production (FIGS. 10B and 10C). CD62L and CD44 expression, BCL6 and CXCR5 expression, and IL-17 production of Irf4^{-/-} TEa cells were less affected by checkpoint blockade (FIG. 11B), suggesting that Irf4^{-/-} TEa cells exposed to checkpoint blockade remained different from WT TEa cells. Taken together, the combined blockade of PD-L1 and CTLA-4 was effective to reverse the initial dysfunction of Irf4-deficient CD4+ T cells by restoring their ability to proliferate and secrete IFN-γ.

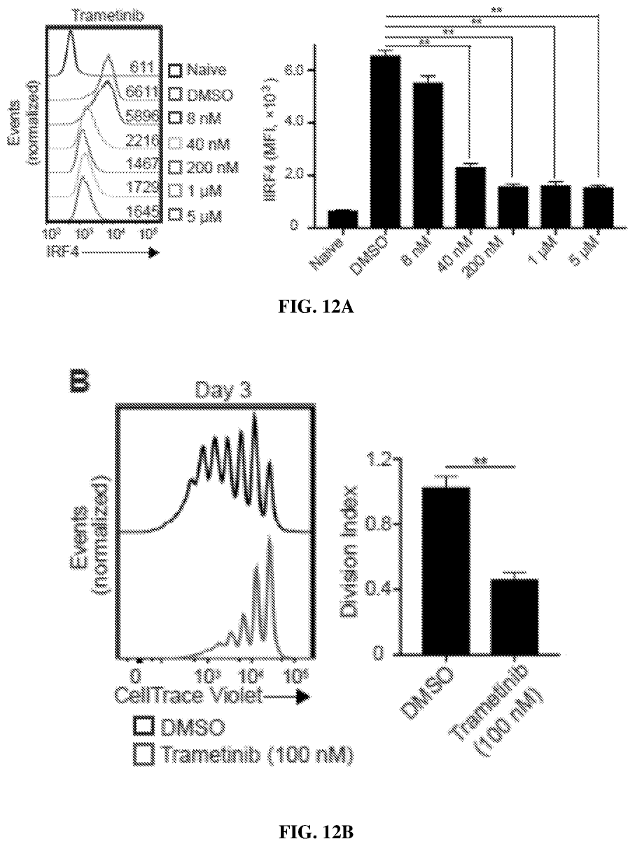

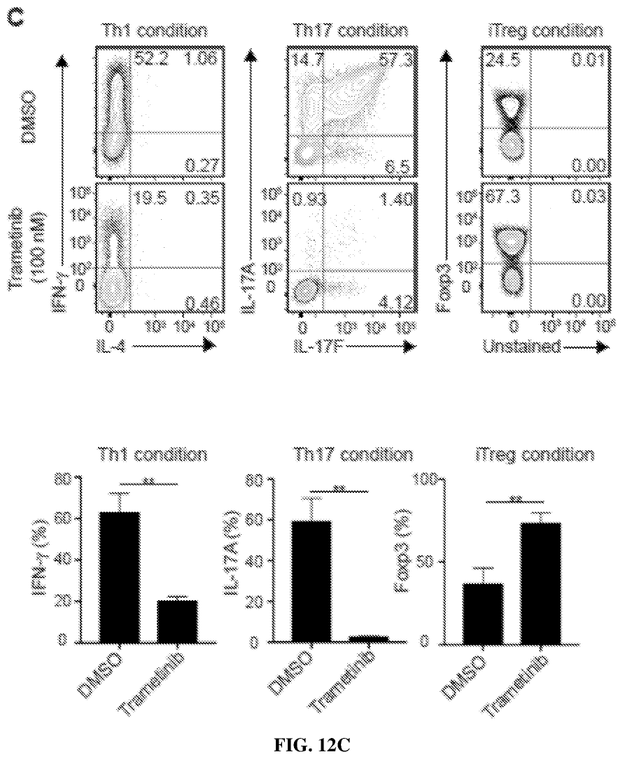

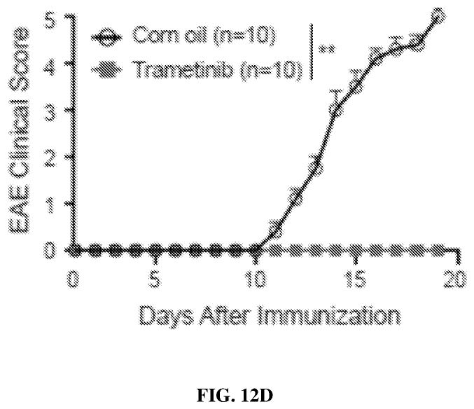

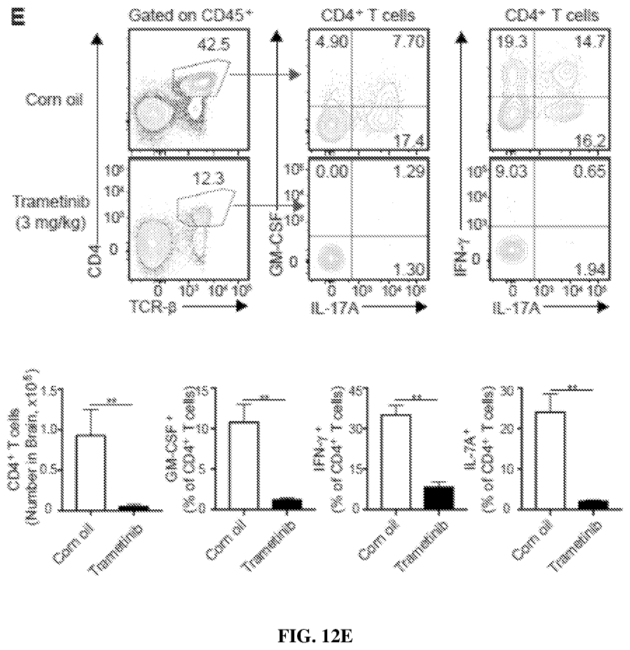

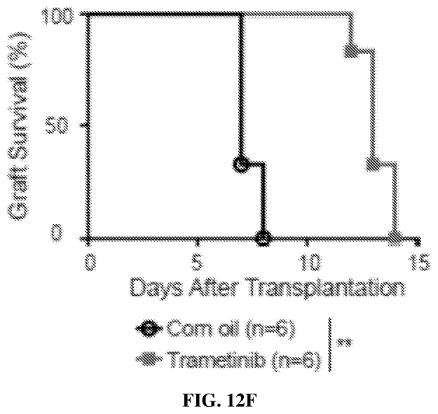

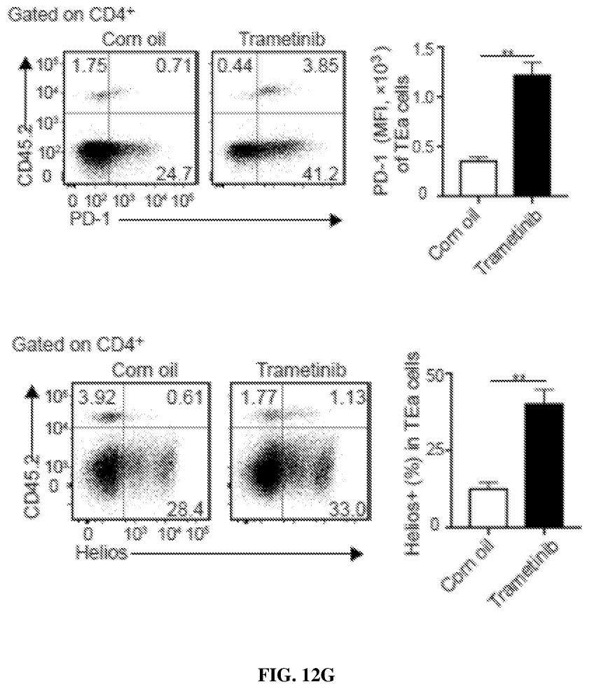

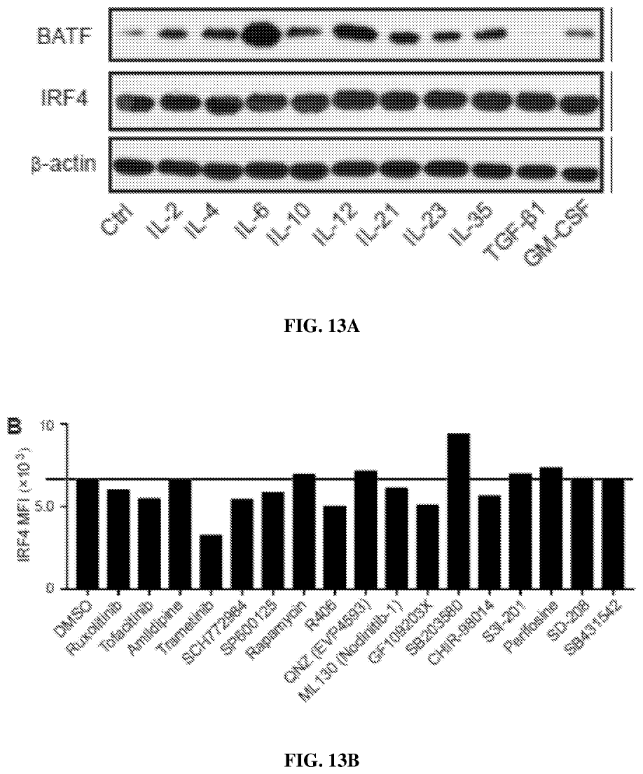

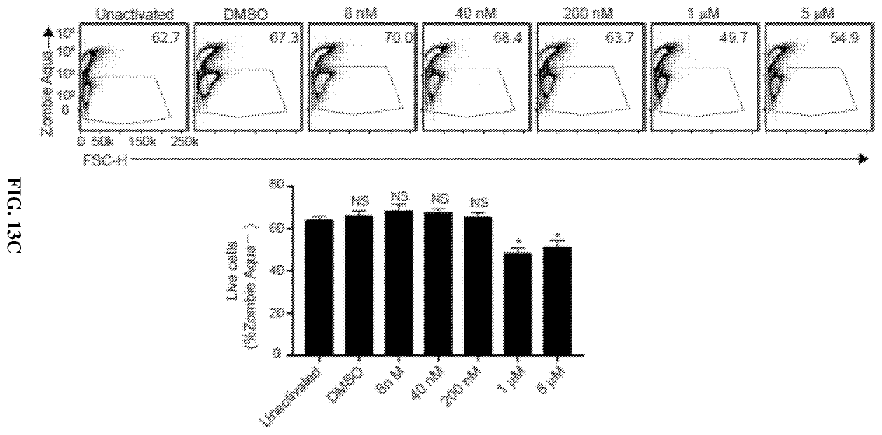

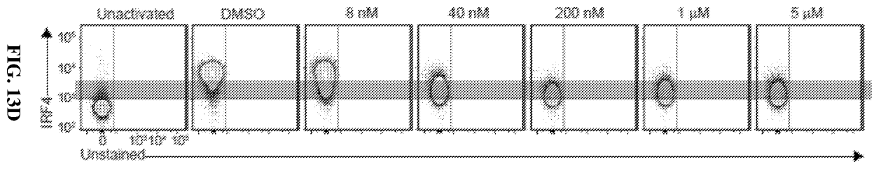

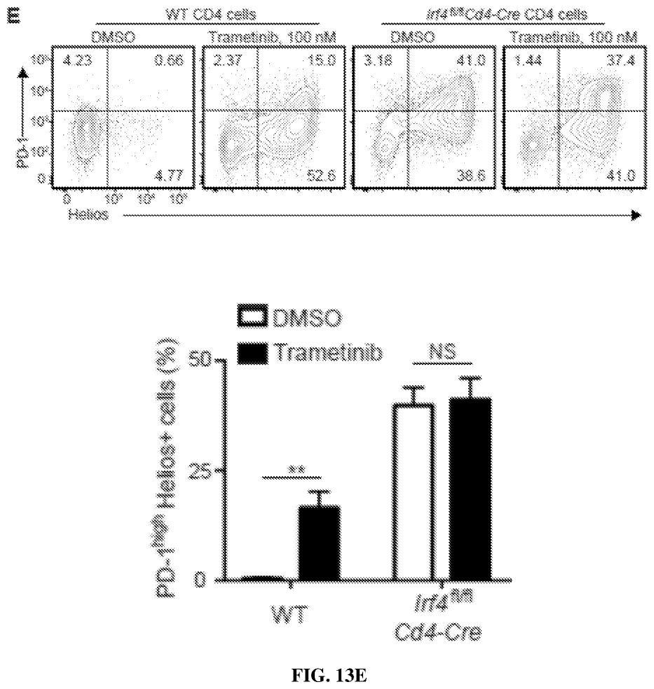

Pharmacological inhibition of IRF4 reduces Th1 and Th17 cell differentiation, abrogates EXE development and prolongs heart allograft survival. IRF4 deletion led to CD4+ T cell dysfunction in transplantation, indicating the therapeutic use of IRF4 inhibition in preventing allograft rejection. To identify approaches for IRF4 inhibition, the effects of cytokines and small-molecule inhibitors on IRF4 expression upon in vitro CD4+ T cell activation were examined. None of the tested cytokines modulated the IRF4 expression in activated CD4+ T cells, though several cytokines altered the expression of a binding partner of IRF4, basic leucine zipper transcription factor ATF-like (BATF) (FIG. 13A). Many tested inhibitors induced T cell death upon in vitro activation and reduced IRF4 expression (data not shown). Nevertheless, by gating on the living CD4+ T cells, a MEK1/2 inhibitor trametinib most potently reduced IRF4 expression in a dose-dependent manner (FIGS. 12A, 13B, 13C, and 13D). Similar to Irf4-deficient CD4+ T cells, the majority of the trametinib-treated WT CD4+ T cells expressed Helios (which was not expressed by most DMSO-treated WT CD4+ T cells). Trametinib also increased PD-1 expression on WT CD4+ T cells, and most PD-1^{hi} cells were Helios positive. For activated Irf4-deficient CD4+ T cells, PD-1 and Helios were already highly expressed, and trametinib barely affected their expression (FIG. 13E). Moreover, 100 nM trametinib was sufficient to inhibit CD4+ T cell proliferation as well as Th1 and Th17 cell differentiation, but promoted the differentiation of inducible Treg cells (FIGS. 12B and 12C).

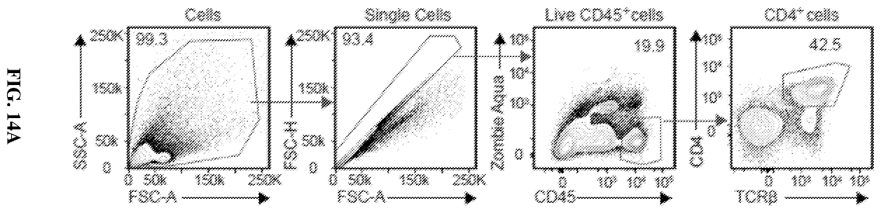

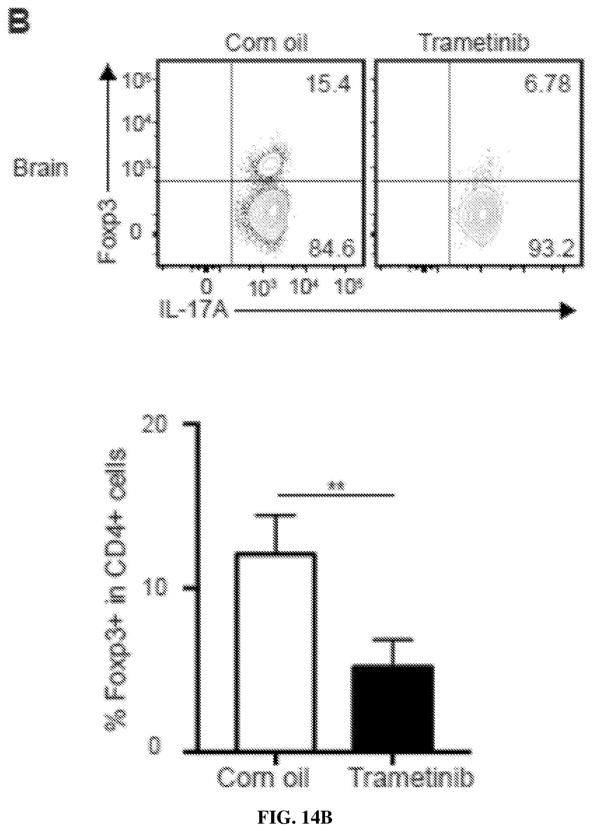

Th1 and Th17 cells contribute to the pathogenesis of experimental autoimmune encephalomyelitis (EAE) (Rangachari and Kuchroo, 2013). Thus, it was determined whether transient trametinib treatment affected EAE development. Female WT mice were immunized with the MOG35-55 peptide and treated with corn oil or 3 mg/kg trametinib every other day from day 0 to day 12. Control mice treated with corn oil developed EAE with a mean disease onset of 12.0±0.47 days post-immunization. By contrast, mice treated with trametinib were resistant to EAE and exhibited no incidence of disease, indicated by absence in the loss of motor function or paralysis throughout the entire period of observation (FIG. 12D). CD4+ T cell infiltration in the brain was dramatically decreased in mice treated with trametinib compared with corn oil controls, and the expression of cytokines GM-CSF, IFN-γ, IL-17, and Foxp3 by brain infiltrating CD4+ T cells was significantly decreased (FIGS. 12E, 14A, and 14B). The frequency of IL-17+CD4+ T cells in the periphery was also lower in trametinib-treated mice than that of control mice (FIG. 14C).

It was then determined whether trametinib was effective to prolong allograft survival. WT B6 mice were transplanted Balb/c hearts and treated with corn oil or 3 mg/kg trametinib every other day from day 0 to day 12. Trametinib-treated recipient mice displayed prolonged heart allograft survival (MST=13.17±0.75 days) compared with corn oil controls (MST=7.33±0.52 days) (FIG. 12F). A TEa cell transfer model was applied to determine whether or not trametinib affects the PD-1 and Helios expressions of alloantigen-specific CD4+ T cells. CD45.1+B6 mice were transferred with CD45.2+ TEa cells on day −1, transplanted with Balb/c hearts on day 0, and treated with corn oil or 3 mg/kg trametinib on day 0, 2, 4, and 6. On day 7 post-transplant, splenocytes were analyzed. Trametinib promoted PD-1 and Helios expression on TEa cells in transplanted mice (FIG. 12G). Collectively, these data show that trametinib effectively reduced IRF4 expression in activated T cells, inhibited Th1 and Th17 cell differentiation, abrogated EAE development, and prolonged heart allograft survival.

### Discussion

The transcriptional programs regulating allogeneic T cell responses and subsequent transplant outcomes (rejection versus acceptance) have not been adequately explored. In the present work, all heart allografts survived throughout the observation period in mice when IRF4 was deleted in T cells. Further mechanistic investigation revealed that IRF4 repressed the expression of a group of molecules associated with T cell dysfunction, including PD-1 and Helios. Thus, Irf4-deficient CD4+ T cells displayed increased expression of these molecules and underwent differentiation to a dysfunctional fate. The initial dysfunctional state of Irf4-deficient T cells was reversible by immune checkpoint blockade, but its late dysfunctional state was irreversible under the methods tested. Therefore, IRF4 is a key transcriptional determinant that regulates effector T cell fate decisions; IRF4 deletion leads to progressive establishment of CD4+ T cell dysfunction and long-term allograft survival.

All Balb/c heart allografts survived throughout the observation period in Irf4^{fl/fl}Cd4-Cre mice that were not treated with any immunosuppressive therapies. Of note, deficiency of several other molecules also allows T cell development but impairs T cell function, such as BATF, signal transducer and activator of transcription 3 (STAT3), nuclear factor κB subunit c-Rel, and IL2-inducible T cell kinase (Itk) (Murphy et al., 2013; Notarangelo, 2013). BATF and STAT3 are also required for Th17 and Tfh cell differentiation. Balb/c hearts were transplanted into Batf^{-/-}, Irf4^{-/-}, and Stat3^{fl/fl}Cd4-Cre mice (data not shown). Only Irf4^{-/-} and Irf4^{fl/fl}Cd4-Cre recipient mice permanently accepted Balb/c hearts throughout the observation period. Thus, lack of Th17 and Tfh cell differentiation is not sufficient to completely explain the severe dysfunction of Irf4-deficient T cells. c-Rel and Itk have been shown to control IRF4 expression in lymphocytes (Grumont and Gerondakis, 2000; Nayar et al., 2012). The role of these two molecules in transplantation was either unknown or less significant than that of IRF4 observed in this study (Yang et al., 2002).

Previous reports showed that Irf4-deficient T cells do not expand properly upon antigen stimulation (Mahnke et al., 2016; Man et al., 2013). T cell frequencies in the spleens of Irf4^{fl/fl}Cd4-Cre mice remained largely unchanged at day 7 post-transplantation. Thus, the expansion of alloreactive T cells in those recipients was impaired. Alloantigen-specific Irf4^{-/-} CD4+ T cells responded to heart allografts and expanded as indicated by CTV labeling, though they were generally found to be 2 to 4-fold less than the frequency of expanded WT alloantigen-specific CD4+ T cells. It took as few as 2 million transferred WT CD4+ T cells to induce acute rejection of heart allografts in Irf4^{fl/fl}Cd4-Cre recipients. By contrast, whole endogenous population of Irf4^{fl/fl}Cd4-Cre T cells plus 20 million transferred Irf4-deficient CD4+ T cells failed to reject heart allografts in Irf4^{fl/fl}Cd4-Cre recipients. These results showed that reduced expansion of Irf4-deficient CD4+ T cells was not the only reason why Irf4^{fl/fl}Cd4-Cre recipients permanently accepted their heart allografts.

A surprising, major mechanistic finding in this study was that IRF4 repressed the expression of essential molecules associated with T cell dysfunction. Hence, IRF4 deletion in activated CD4+ T cells led to increased expression of PD-1, Helios, PLAGL1, CD160, CD73, and FR4, which are characteristic markers of dysfunctional T cells (Crawford et al., 2014; Fathman and Lineberry, 2007; Martinez et al., 2012; Wherry and Kurachi, 2015). Given that PD-1 is a key immune checkpoint molecule implicated in T cell dysfunction, the regulation of PD-1 expression by IRF4 was investigated. In the absence of IRF4, activated CD4+ T cells induced an active chromatin state at the cis-elements of Pdcd1, as well as increased Helios expression. Helios was found to bind to cis-elements of Pdcd1 and promote PD-1 expression. Helios has been shown to be highly expressed by natural Treg cells, exhausted T cells, and tolerated T cells (Crawford et al., 2014; Kim et al., 2015; Ross et al., 2014; Singer et al., 2016). It will be interesting to further define whether Helios sustains PD-1 expression in those cells. Transcription factors NFAT1, T-bet, FoxO1, and Blimp1 have also been shown to regulate PD-1 expression (Wherry and Kurachi, 2015), but the connection between IRF4 and these molecules on Pdcd1 transcription is unknown.

Initial dysfunction of Irf4-deficient T cells in transplantation can be attributed to the presence of inhibitory checkpoints. PD-1 was highly expressed on alloantigen-specific Irf4-deficient T cells; blockade of its ligand, PD-L1, during the initial days post-transplant was capable of restoring the anti-allograft function of Irf4-deficient T cells. Activated Irf4-deficient T cells also expressed intracellular CTLA-4, though at a relatively low expression compared to that of activated WT T cells. Transient blockade of PD-L1 plus CTLA-4 during the initial days post-transplant synergistically and completely rescued the anti-allograft function of Irf4-deficient T cells by restoring their ability to undergo proliferation and IFN-γ production. Thus, these two major inhibitory checkpoints operated as non-redundant pathways to mediate the dysfunction of Irf4-deficient T cells, likely due to their distinct mechanisms of action (Baumeister et al., 2016; Larkin et al., 2015; Postow et al., 2015). However, blockade of PD-L1 plus CTLA-4 starting from day 30 post-transplant did not induce heart allograft rejection in Irf4^{fl/fl}Cd4-Cre recipients. Therefore, a progressive process likely exists to establish the irreversible dysfunctional state of Irf4-deficient T cells.

A very high proportion of polyclonal WT T cells (˜0.1-10%) in transplant recipients are able to recognize and respond to intact allogeneic MHC on donor APCs (termed direct allorecognition) or donor MHC peptides presented by recipient APCs (termed indirect allorecognition) (Lechler et al., 2005; Rogers and Lechler, 2001). These unique characteristics of allogeneic T cell response contribute significantly to allograft rejection. TEa CD4+ T cells only recognize a Balb/c donor I-Ea allopeptide through the indirect allorecognition pathway, and may be less effective to mediate transplant rejection compared to that of polyclonal WT CD4+ T cells. Thus, 20 million WT TEa and Irf4^{-/-} TEa cells were adoptively transferred into Irf4^{fl/fl}Cd4-Cre recipients to investigate their ability to reject heart allografts. WT TEa but not Irf4^{-/-} TEa cells mediated heart allograft rejection. Therefore, the TEa cell transfer model can track the in vivo phenotypic changes of alloreactive CD4+ T cells associated with IRF4 deficiency and immune checkpoint blockade.

Recent reports indicated that in patients with stable function of transplanted kidneys but also developed metastatic cancer, PD-1 blockade was used to treat cancer. Unfortunately, PD-1 blockade triggered acute rejection of the transplanted kidneys (Alhamad et al., 2016; Lipson et al., 2016). Trametinib was an effective inhibitor that diminished IRF4 expression in activated CD4+ T cells. Transient trametinib treatment reduced EAE development and prolonged allograft survival. These results demonstrated the feasibility of targeting IRF4 expression for eliminating undesired T cell responses.

In summary, IRF4 restrained the dysfunctional differentiation of CD4+ T cells, representing a novel role for IRF4 in linking TCR signaling to CD4+ T cell fates. Moreover, the concept of T cell dysfunction, which is mainly documented in chronic viral infection and tumor models, is introduced herein into research aiming to eliminate undesired T cells responses, such as transplant rejection and autoimmunity. IRF4 deficiency resulted in progressive establishment of effector CD4+ T cell dysfunction, and even mice tested herein having fully MHC-mismatched heart allografts had enhanced survival. Induction of T cell dysfunction by targeting IRF4 is a therapeutic strategy for treatment of transplant rejection and autoimmune disorders.

### REFERENCES

Alhamad, T., Venkatachalam, K., Linette, G. P., and Brennan, D. C. (2016). Checkpoint Inhibitors in Kidney Transplant Recipients and the Potential Risk of Rejection. Am. J. Transplant. 16, 1332-1333.
Bally, A. P., Austin, J. W., and Boss, J. M. (2016). Genetic and Epigenetic Regulation of PD-1 Expression. J. Immunol. 196, 2431-2437.
Baumeister, S. H., Freeman, G. J., Dranoff, G., and Sharpe, A. H. (2016). Coinhibitory Pathways in Immunotherapy for Cancer. Annu. Rev. Immunol. 34, 539-573.
Bollig, N., Brustle, A., Kellner, K., Ackermann, W., Abass, E., Raifer, H., Camara, B., Brendel, C., Giel, G., Bothur, E., et al. (2012). Transcription factor IRF4 determines germinal center formation through follicular T-helper cell differentiation. Proc. Natl. Acad. Sci. USA 109, 8664-8669.
Bolton, E. M., Gracie, J. A., Briggs, J. D., Kampinga, J., and Bradley, J. A. (1989). Cellular requirements for renal allograft rejection in the athymic nude rat. J. Exp. Med. 169, 1931-1946.
Brustle, A., Heink, S., Huber, M., Rosenplanter, C., Stadelmann, C., Yu, P., Arpaia, E., Mak, T. W., Kamradt, T., and Lohoff, M. (2007). The development of inflammatory T(H)-17 cells requires interferon-regulatory factor 4. Nat. Immunol. 8, 958-966.
Chen, W., Diao, J., Stepkowski, S. M., and Zhang, L. (2007). Both infiltrating regulatory T cells and insufficient antigen presentation are involved in long-term cardiac xenograft survival. J. Immunol. 179, 1542-1548.
Conlon, T. M., Saeb-Parsy, K., Cole, J. L., Motallebzadeh, R., Qureshi, M. S., Rehakova, S., Negus, M. C., Callaghan, C. J., Bolton, E. M., and Bradley, J. A., et al. (2012). Germinal center alloantibody responses are mediated exclusively by indirect-pathway CD4 T follicular helper cells. J. Immunol. 188, 2643-2652.
Crawford, A., Angelosanto, J. M., Kao, C., Doering, T. A., Odorizzi, P. M., Barnett, B. E., and Wherry, E. J. (2014). Molecular and transcriptional basis of CD4(+) T cell dysfunction during chronic infection. Immunity 40, 289-302.
Cretney, E., Xin, A., Shi, W., Minnich, M., Masson, F., Miasari, M., Belz, G. T., Smyth, G. K., Busslinger, M., Nutt, S. L., et al. (2011). The transcription factors Blimp-1 and IRF4 jointly control the differentiation and function of effector regulatory T cells. Nat. Immunol. 12, 304-311.
Fathman, C. G., and Lineberry, N. B. (2007). Molecular mechanisms of CD4+ T-cell anergy. Nat. Rev. Immunol. 7, 599-609.
Grumont, R. J., and Gerondakis, S. (2000). Rel induces interferon regulatory factor 4 (IRF-4) expression in lymphocytes: modulation of interferon-regulated gene expression by rel/nuclear factor kappaB. J. Exp. Med. 191, 1281-1292.
Grusdat, M., McIlwain, D. R., Xu, H. C., Pozdeev, V. I., Knievel, J., Crome, S. Q., Robert-Tissot, C., Dress, R. J., Pandyra, A. A., Speiser, D. E., et al. (2014). IRF4 and BATF are critical for CD8(+) T-cell function following infection with LCMV. Cell Death Differ. 21, 1050-1060.
Gupta, S., Balasubramanian, S., Thornley, T. B., Strom, T. B., and Kenny, J. J. (2011). Direct pathway T-cell alloactivation is more rapid than indirect pathway alloactivation. Transplantation 91, e65-67.
Huber, M., Brustle, A., Reinhard, K., Guralnik, A., Walter, G., Mahiny, A., von Löw, E., and Lohoff, M. (2008). IRF4 is essential for IL-21-mediated induction, amplification, and stabilization of the Th17 phenotype. Proc. Natl. Acad. Sci. USA 105, 20846-20851.
Huber, M., and Lohoff, M. (2014). IRF4 at the crossroads of effector T-cell fate decision. Eur. J. Immunol. 44, 1886-1895.
Kim, H. J., Barnitz, R. A., Kreslavsky, T., Brown, F. D., Moffett, H., Lemieux, M. E., Kaygusuz, Y., Meissner, T., Holderried, T. A., Chan, S., et al. (2015). Stable inhibitory activity of regulatory T cells requires the transcription factor Helios. Science 350, 334-339.
Krieger, N. R., Yin, D. P., and Fathman, C. G. (1996). CD4+ but not CD8+ cells are essential for allorejection. J. Exp. Med. 184, 2013-2018.
Larkin, J., Chiarion-Sileni, V., Gonzalez, R., Grob, J. J., Cowey, C. L., Lao, C. D., Schadendorf, D., Dummer, R., Smylie, M., Rutkowski, P., et al. (2015). Combined Nivolumab and Ipilimumab or Monotherapy in Untreated Melanoma. N. Engl. J. Med. 373, 23-34.
Lechler, R. I., Sykes, M., Thomson, A. W., and Turka, L. A. (2005). Organ transplantation-how much of the promise has been realized? Nat. Med. 11, 605-613.
Lee, Y., Awasthi, A., Yosef, N., Quintana, F. J., Xiao, S., Peters, A., Wu, C., Kleinewietfeld, M., Kunder, S., Hafler, D. A., et al. (2012). Induction and molecular signature of pathogenic TH17 cells. Nat. Immunol. 13, 991-999.
Li, P., Spolski, R., Liao, W., Wang, L., Murphy, T. L., Murphy, K. M., and Leonard, W. J. (2012). BATF-JUN is critical for IRF4-mediated transcription in T cells. Nature 490, 543-546.
Lipson, E. J., Bagnasco, S. M., Moore, J., Jr., Jang, S., Patel, M. J., Zachary, A. A., Pardoll, D. M., Taube, J. M., Drake, C. G. (2016). Tumor Regression and Allograft Rejection after Administration of Anti-PD-1. N. Engl. J. Med. 374, 896-898.
Liu, Z., Fan, H., and Jiang, S. (2013). CD4(+) T-cell subsets in transplantation. Immunol Rev, 252, 183-191.
Mahnke, J., Schumacher, V., Ahrens, S., Kading, N., Feldhoff, L. M., Huber, M., Rupp, J., Raczkowski, F., and Mittrucker, H. W. (2016). Interferon Regulatory Factor 4 controls TH1 cell effector function and metabolism. Sci. Rep. 6, 35521.
Man, K., Miasari, M., Shi, W., Xin, A., Henstridge, D. C., Preston, S., Pellegrini, M., Belz, G. T., Smyth, G. K., Febbraio, M. A., et al. (2013). The transcription factor IRF4 is essential for TCR affinity-mediated metabolic programming and clonal expansion of T cells. Nat. Immunol. 14, 1155-1165.
Martinez, R. J., Zhang, N., Thomas, S. R., Nandiwada, S. L., Jenkins, M. K., Binstadt, B. A., and Mueller, D. L. (2012). Arthritogenic self-reactive CD4+ T cells acquire an FR4hiCD73hi anergic state in the presence of Foxp3+ regulatory T cells. J. Immunol. 188, 170-181.
Mittrucker, H. W., Matsuyama, T., Grossman, A., Kundig, T. M., Potter, J., Shahinian, A., Wakeham, A., Patterson, B., Ohashi, P. S., and Mak, T. W. (1997). Requirement for the transcription factor LSIRF/IRF4 for mature B and T lymphocyte function. Science 275, 540-543.
Miyahara, Y., Khattar, M., Schroder, P. M., Mierzejewska, B., Deng, R., Han, R., Hancock, W. W., Chen, W., and Stepkowski, S. M. (2012). Anti-TCRbeta mAb induces long-term allograft survival by reducing antigen-reactive T cells and sparing regulatory T cells. Am. J. Transplant. 12, 1409-1418.
Murphy, T. L., Tussiwand, R., and Murphy, K. M. (2013). Specificity through cooperation: BATF-IRF interactions control immune-regulatory networks. Nat. Rev. Immunol. 13, 499-509.
Nayar, R., Enos, M., Prince, A., Shin, H., Hemmers, S., Jiang, J. K., Klein, U., Thomas, C. J., and Berg, L. J. (2012). TCR signaling via Tec kinase ITK and interferon regulatory factor 4 (IRF4) regulates CD8+ T-cell differentiation. Proc. Natl. Acad. Sci. USA 109, E2794-2802.
Notarangelo, L. D. (2013). Functional T cell immunodeficiencies (with T cells present). Annu. Rev. Immunol 31, 195-225.
Ochiai, K., Maienschein-Cline, M., Simonetti, G., Chen, J., Rosenthal, R., Brink, R., Chong, A. S., Klein, U., Dinner, A. R., Singh, H., et al. (2013). Transcriptional regulation of germinal center B and plasma cell fates by dynamical control of IRF4. Immunity 38, 918-929.
Postow, M. A., Chesney, J., Pavlick, A. C., Robert, C., Grossmann, K., McDermott, D., Linette, G. P., Meyer, N., Giguere, J. K., Agarwala, S. S., et al. (2015). Nivolumab and ipilimumab versus ipilimumab in untreated melanoma. N. Engl. J. Med. 372, 2006-2017.
Rangachari, M., and Kuchroo, V. K. (2013). Using EAE to better understand principles of immune function and autoimmune pathology. J. Autoimmun. 45, 31-39.
Rogers, N. J., and Lechler, R. I. (2001). Allorecognition. Am. J. Transplant. 1, 97-102.
Ross, E. M., Bourges, D., Hogan, T. V., Gleeson, P. A., and van Driel, I. R. (2014). Helios defines T cells being driven to tolerance in the periphery and thymus. Eur. J. Immunol. 44, 2048-2058.
Safinia, N., Scotta, C., Vaikunthanathan, T., Lechler, R. I., and Lombardi, G. (2015). Regulatory T Cells: Serious Contenders in the Promise for Immunological Tolerance in Transplantation. Front. Immunol. 6, 438.
Schietinger, A., and Greenberg, P. D. (2014). Tolerance and exhaustion: defining mechanisms of T cell dysfunction. Trends Immunol. 35, 51-60.
Schietinger, A., Philip, M., Krisnawan, V. E., Chiu, E. Y., Delrow, J. J., Basom, R. S., Lauer, P., Brockstedt, D. G., Knoblaugh, S. E., Hammerling, G. J., et al. (2016). Tumor-Specific T Cell Dysfunction Is a Dynamic Antigen-Driven Differentiation Program Initiated Early during Tumorigenesis. Immunity 45, 389-401.
Singer, M., Wang, C., Cong, L., Marjanovic, N. D., Kowalczyk, M. S., Zhang, H., Nyman, J., Sakuishi, K., Kurtulus, S., Gennert, D., et al. (2016). A Distinct Gene Module for Dysfunction Uncoupled from Activation in Tumor-Infiltrating T Cells. Cell 166, 1500-1511.
Staudt, V., Bothur, E., Klein, M., Lingnau, K., Reuter, S., Grebe, N., Gerlitzki, B., Hoffmann, M., Ulges, A., Taube, C., et al. (2010). Interferon-regulatory factor 4 is essential for the developmental program of T helper 9 cells. Immunity 33, 192-202.
Vander Lugt, B., Khan, A. A., Hackney, J. A., Agrawal, S., Lesch, J., Zhou, M., Lee, W. P., Park, S., Xu, M., DeVoss, J., et al. (2014). Transcriptional programming of dendritic cells for enhanced MHC class II antigen presentation. Nat. Immunol. 15, 161-167.
Wherry, E. J., and Kurachi, M. (2015). Molecular and cellular insights into T cell exhaustion. Nat. Rev. Immunol. 15, 486-499.
Xiao, X., Shi, X., Fan, Y., Wu, C., Zhang, X., Minze, L., Liu, W., Ghobrial, R. M., Lan, P., and Li, X. C. (2016). The Costimulatory Receptor OX40 Inhibits Interleukin-17 Expression through Activation of Repressive Chromatin Remodeling Pathways. Immunity 44, 1271-1283.
Yang, H., Thomas, D., Boffa, D. J., Ding, R., Li, B., Muthukumar, T., Sharma, V. K., Lagman, M., Luo, G. X., Kapur, S., et al. (2002). Enforced c-REL deficiency prolongs survival of islet allografts1. Transplantation 74, 291-298.
Yao, S., Buzo, B. F., Pham, D., Jiang, L., Taparowsky, E. J., Kaplan, M. H., and Sun, J. (2013). Interferon regulatory factor 4 sustains CD8(+) T cell expansion and effector differentiation. Immunity 39, 833-845.
Yuan, X., Paez-Cortez, J., Schmitt-Knosalla, I., D'Addio, F., Mfarrej, B., Donnarumma, M., Habicht, A., Clarkson, M. R., Iacomini, J., Glimcher, L. H., et al. (2008). A novel role of CD4 Th17 cells in mediating cardiac allograft rejection and vasculopathy. J. Exp. Med. 205, 3133-3144.
Zarour, H. M. (2016). Reversing T-cell Dysfunction and Exhaustion in Cancer. Clin. Cancer Res. 22, 1856-1864.
Zheng, Y., Chaudhry, A., Kas, A., deRoos, P., Kim, J. M., Chu, T. T., Corcoran, L., Treuting, P., Klein, U., and Rudensky, A. Y. (2009). Regulatory T-cell suppressor program co-opts transcription factor IRF4 to control T(H)2 responses. Nature 458, 351-356.

### Example 2

Achieving transplant tolerance remains the ultimate goal in the field of organ transplantation. Described is reduction of transcription factor interferon regulatory factor 4 (IRF4) in T Cells induced heart transplant acceptance by driving allogeneic CD4⁺ T cell dysfunction. In this example, it is shown that heart-transplanted mice with T cell-specific IRF4 deletion were tolerant to donor-specific antigens and accepted subsequently transplanted donor-type but not third-party skin allografts. Immune checkpoint blockade during initial days post-heart grafting transiently reinvigorated Irf4-deficient T cells and triggered heart allograft rejection in recipient mice with T cell-specific IRF4 deletion, but reinvigoration was progressively lost and transplant tolerance was re-established within 30 days. By tracking alloantigen-specific CD4⁺ T cells in vivo, it was revealed that checkpoint blockade restored expression levels of the majority of wild-type T cell-expressed genes in Irf4-deficient T cells, indicating the reinvigoration of Irf4-deficient T cells. Nevertheless, the remaining un-restored genes following checkpoint blockade may be responsible for the reinvigorated Irf4-deficient T cells to become re-dysfunctional. Hence, targeting IRF4 represents a therapeutic strategy for driving intrinsic T cell dysfunction and achieving alloantigen-specific transplant tolerance.

### Materials and Methods

Mice. Cd4-Cre, Irf4^{flox/flox}, B6.SJL CD45.1 congenic, TEa TCR transgenic, BALB/c, and C57BL/6 (B6) mice were purchased from Jackson Laboratory (Bar Harbor, Mass.). Irf4^{flox/flox} mice were crossed to Cd4-Cre mice to create Irf4^{fl/fl} Cd4-Cre mice. TEa mice were crossed to Irf4^{-/-} mice to create Irf4^{+/+} TEa mice. TCR transgenic (Vα2⁺ Vβ6⁺) CD4⁺ T cells from TEa mice (B6 background; I-A^{b}, I-E⁻) are specific for Balb/c allopeptide I-Eα₅₂₋₆₈ presented by B6 antigen-presenting cells and bound to I-A^{b}. TEa mice were crossed to CD45.1 congenic mice to create (TEa×CD45.1)F1 mice, in which leucocytes are CD45.1⁺ CD45.2⁺. Mice were housed in a specific pathogen free facility at Houston Methodist Research Institute in Houston, Tex. All animal experiments in this study were approved by the Houston Methodist Animal Care Committee in accordance with institutional animal care and use guidelines.

Murine heterotopic heart transplantation. Hearts from BALB/c donors were transplanted into 8 to 10-week-old male Irf4^{fl/fl}Cd4-Cre or CD45.1⁺ congenic mice by a previously described method (8). In brief, blood flow of the abdominal aorta and inferior vena cava in anesthetized recipient was interrupted by ligation with 6-0 silk thread. Incisions were made in recipient abdominal aorta and inferior vena cava to perform anastomosis with donor heart aorta and donor pulmonary artery, respectively. Anastomosis was made by 11-0 sutures running continuously. 6-0 silk thread was removed and abdomen was then closed. Some recipient mice were i.p. injected with 400 μg Rat IgG, or 200 μg anti-PD-L1 (clone 10F.9G2) plus 200 μg anti-CTLA-4 (clone 9D9) mAbs (Bio-X-Cell; West Lebanon, N.H.) on days 0, 3, 5 post heart grafting. Heart graft survival was monitored daily by palpation, and the day of complete cessation of heartbeat was considered as the day of rejection.

Murine skin transplantation. BALB/c and C3H skin allografts were transplanted onto BALB/c heart transplanted Irf4^{fl/fl}Cd4-Cre mice by a previously described method (12). In brief, dorsal side of donor ear skins were transplanted on the back of recipient mice. Vaseline gauze was placed directly on the skin grafts. A sterile bandage was then wrapped around the recipient mouse, and was removed at 6-7 days after skin transplantation. Transplanted mice were monitored daily, and >80% necrosis of the donor skin tissue was considered as rejection.

Irf4 transduction and adoptive transfer of Irf4^{-/-} CD4⁺ T cells. cDNA fragments encoding mouse Irf4 were amplified by PCR and then cloned into a pMYs-IRES-EGFP retroviral vector (Cell Biolabs). Retroviral particles were produced by transfecting plat-E cells with those retroviral vectors according to the manufacturer's recommendations (Cell Biolabs). For preparing IRF4-GFP re-introduced and GFP-control (Ctrl) transduced alloreactive Irf4^{-/-} CD4⁺ T cells, Balb/c splenic dendritic cells (DCs) were isolated by using the Pan Dendritic Cell Isolation Kit (Miltenyi Biotec). Irf4^{-/-} CD4⁺ T cells were activated for 3 days with Balb/c splenic DCs and 100 IU IL-2 (PeproTech), and incubated with freshly prepared retroviral particles as mentioned above. Cells were cultured for one day after transduction, and then adoptively transferred into Irf4^{fl/fl}Cd4-Cre mice on day 1 post-heart transplantation.

Adoptive transfer of TEa cells and microarray analysis. Microarray was performed by the Genomic and RNA Profiling Core at Baylor College of Medicine and data generated has been deposited in NCBI's Gene Expression Omnibus with accession number GSE111757. TCR(Vα2⁺Vβ6⁺)CD45.2⁺ CD4⁺ TEa cells were isolated from splenocytes of WT TEa or Irf4^{-/-} TEa mice by a FACSAria flow cytometer (BD Biosciences). B6.SJL CD45.1⁺ congenic mice were adoptively transferred with either 5×10⁶ CD45.2⁺ WT TEa or 5×10⁶ CD45.2⁺ Irf4^{-/-} TEa cells on day −1, and transplanted with Balb/c hearts on day 0. CD45.1⁺ mice transferred with CD45.2⁺ Irf4^{-/-} TEa cells were i.p. injected with either 400 μg Rat IgG or 200 μg anti-PD-L1 plus 200 μg anti-CTLA-4 mAbs on days 0, 3, 5. On day 6, adoptively transferred CD45.2⁺ TEa cells were sorted from splenocytes of transplant recipients by the FACSAria flow cytometer. Total RNA was extracted from sorted cells with RNeasy mini kit (Qiagen). RNA was quantified using a NanoDrop-1000 spectrophotometer and quality was monitored with the Agilent 2100 Bioanalyzer (Agilent Technologies). Cyanine-3 labeled cRNA was prepared from 0.5 μg RNA using the One-Color Low RNA Input Linear Amplification PLUS kit (Agilent Technologies) and hybridized to Agilent SurePrint G3 Mouse GE v2 8x60K Microarray (G4852B; 074809). Slides were scanned immediately after washing on the Agilent Technologies Scanner (G2505C). Data were normalized and analyzed using the Subio Platform (Subio).

Tracking of adoptively transferred TEa cells. CD45.1⁺ congenic mice were adoptively transferred with mixed splenocytes containing 7.5×10⁶ CD45.1⁺ CD45.2⁺ WT TEa cells [from (TEa×CD45.1)F1 mice] and 7.5×10⁶ CD45.2⁺ Irf4^{+/+} TEa cells (from Irf4^{-/-} TEa mice) on day −1, and transplanted with BALB/c hearts on day 0. Some CD45.1⁺ recipient mice were also i.p. injected with 200 μg anti-PD-L1 plus 200 μg anti-CTLA-4 mAbs on days 0, 3, 5. TEa cells in peripheral blood and spleens of transplant recipients were analyzed on the LSRFortessa flow cytometer (Beckton Dickinson). Fluorochrome-conjugated antibodies were purchased from BioLegend or eBioscience. Zombie Aqua™ Fixable Viability Kit was purchased from BioLegend. Intracellular staining method was previously described (8).

Statistical analysis. Data were represented as mean±SD and analyzed with Prism version 7.0a (GraphPad Software). Other measurements were performed using unpaired Student's t-test. The P values of skin graft survival were determined by the Mann-Whitney test. Differences were considered significant when P<0.05.

### Results

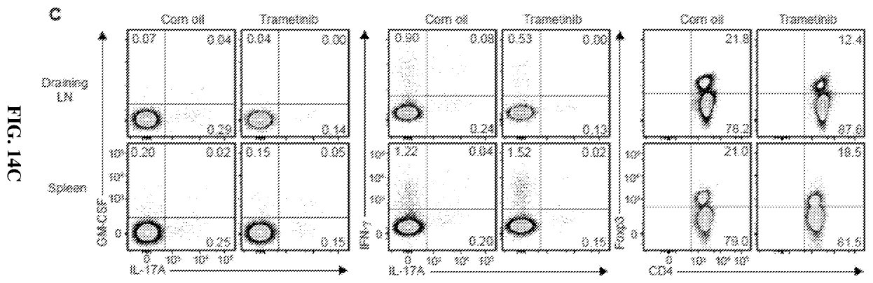

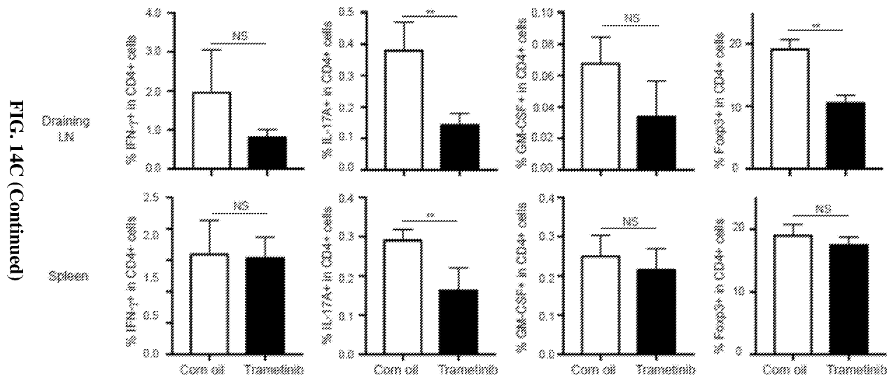

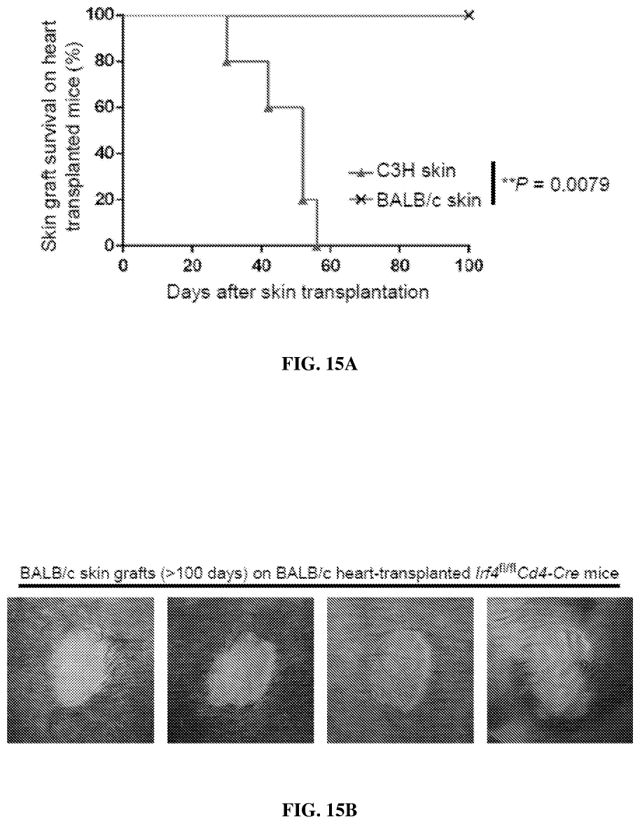

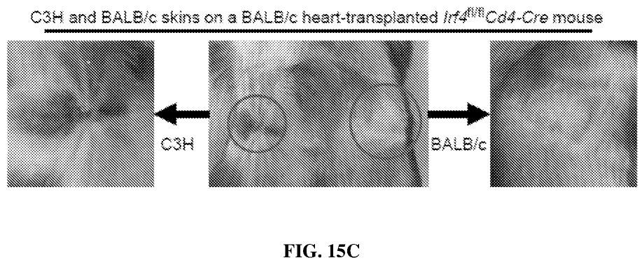

Reduction of IRF4 in T Cells abrogates their ability to reject donor-type skin grafts in heart transplanted recipients. Example 1 describes that alloreactive T cell dysfunction can be achieved in Irf4^{fl/fl}Cd4-Cre mice after heart transplantation (8). To determine whether induced T cell dysfunction affects the survival of subsequently transplanted skin allografts, Irf4^{fl/fl}Cd4-Cre recipients were first transplanted with BALB/c hearts and then transplanted with BALB/c and C3H skin allografts 30 days later. All heart allografts were permanently accepted as previously described (8). Importantly, none of the heart-transplanted Irf4^{fl/fl}Cd4-Cre mice rejected the subsequently transplanted BALB/c skins (mean survival time (MST) of >100 days; n=5) (FIGS. 15A-15C). By contrast, all C3H skin allografts were rejected within 60 days (MST=46.4±10.53 days; n=5) (FIGS. 15A and 15C). Irf4^{fl/fl}Cd4-Cre mice without BALB/c heart transplantation were also capable of rejecting BALB/c skin grafts (MST=31.0±8.16 days; n=4) (data not shown). Hence, selective ablation of IRF4 in T cells abrogated their ability to reject heart allografts and subsequently transplanted donor-type skins.

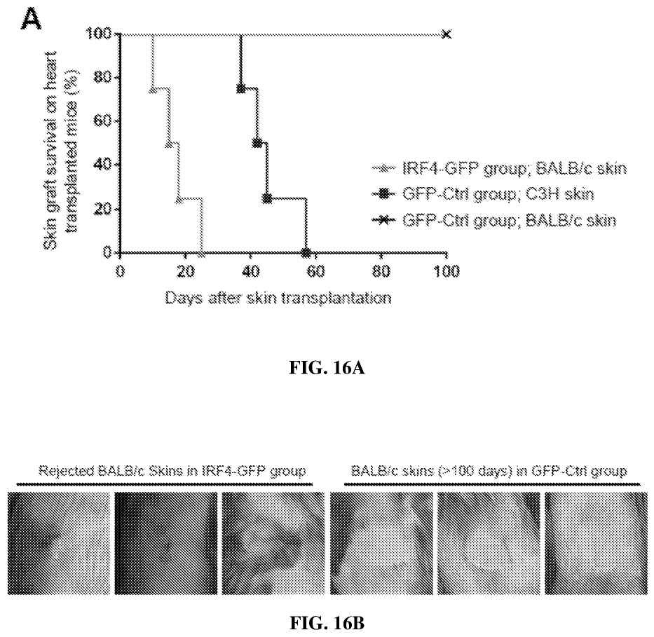

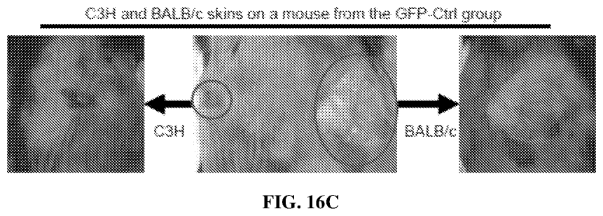

Adoptive transfer of IRF4 re-introduced Irf4^{-/-} CD4⁺ T cells inhibits the induction of transplant tolerance in mice with T cell-specific IRF4 deletion. One approach that has been applied in restoring heart transplant rejection in Irf4^{fl/fl}Cd4-Cre mice was to transfer IRF4 re-introduced Irf4^{-/-} CD4⁺ T cells. Irf4^{-/-} CD4⁺ T cells were stimulated in vitro with allogenic BALB/c splenic DCs and IL-2 for 3 days, followed by transduction with IRF4-GFP or GFP-Ctrl retrovirus for 1 day. Irf4^{fl/fl}Cd4-Cre recipients injected with one million IRF4 re-introduced, but not GFP-Ctrl transduced, Irf4^{-/-} CD4⁺ T cells acutely rejected heart allografts within 6 days, as previously described (8). Recipient mice were transplanted again with skin allografts 30 days after heart grafting. As shown in FIGS. 16A-16C), BALB/c skins were acutely rejected on heart-transplanted recipients that were transferred with IRF4 re-introduced Irf4^{-/-} CD4⁺ T cells (IRF4-GFP cell transfer group; BALB/c skin; MST=17.0±6.27 days; n=4), but were accepted on heart-transplanted recipients that were transferred with GFP-Ctrl transduced Irf4^{-/-} CD4⁺ T cells (GFP-Ctrl cell transfer group; BALB/c skin; MST of >100 days; n=4). C3H skins were rejected within 60 days on heart-transplanted recipients that were transferred with GFP-Ctrl transduced Irf4^{-/-} CD4⁺ T cells (GFP-Ctrl cell transfer group; C3H skin; MST=45.3±8.50 days; n=4). Therefore, adoptive transfer of IRF4 re-introduced, but not GFP-Ctrl transduced, Irf4^{-/-} CD4⁺ T cells inhibits the induction of transplant tolerance in heart-transplanted Irf4^{fl/fl}Cd4-Cre recipients.

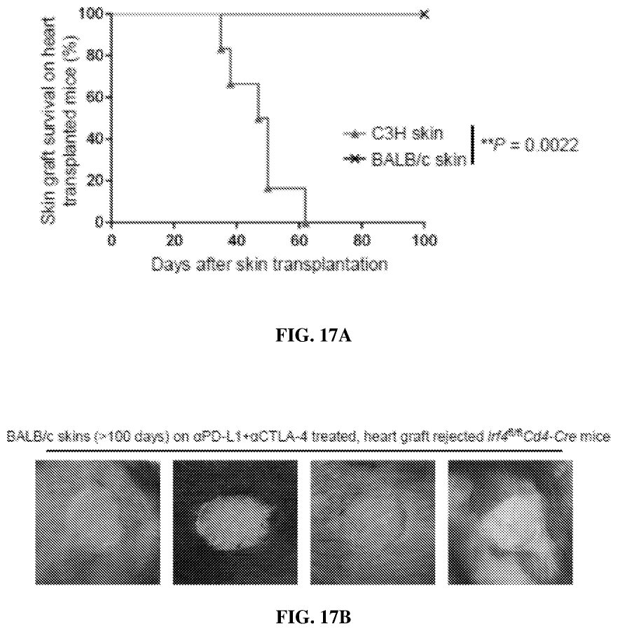

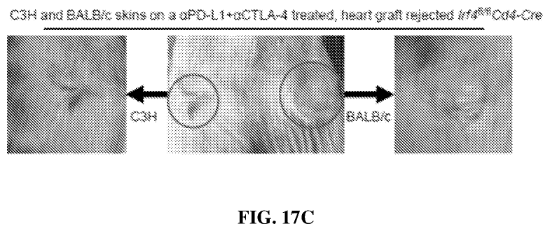

Immune checkpoint blockade induces heart transplant rejection but does not prevent the later establishment of transplant tolerance in mice with T cell-specific IRF4 deletion. Immune checkpoint blockade restored acute heart transplant rejection in Irf4^{fl/fl}Cd4-Cre mice (8). The influence of initial checkpoint blockade-mediated heart allograft rejection on the survival of subsequently transplanted skin allografts was investigated. BALB/c hearts were transplanted into Irf4^{fl/fl}Cd4-Cre mice and treated with anti-PD-L1 and anti-CTLA-4 mAbs on days 0, 3, and 5 post-heart grafting. All heart allografts were acutely rejected within 8 days as previously described (8). Recipients with rejected heart allografts were then transplanted again with skin allografts 30 days after heart grafting. Strikingly, as shown in FIGS. 17A-17C, all BALB/c skins were accepted on those recipients (MST of >100 days; n=6), while all C3H skins were rejected within 60 days (MST=47.0±9.67 days; n=6). Therefore, checkpoint blockade induced acute heart transplant rejection in Irf4^{fl/fl}Cd4-Cre recipients, but did not prevent the later establishment of transplant tolerance.

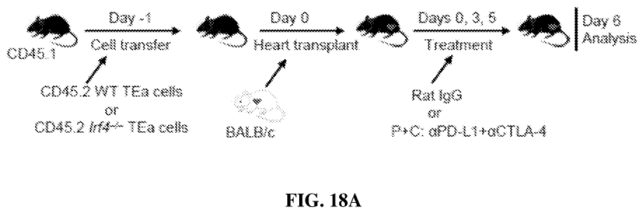

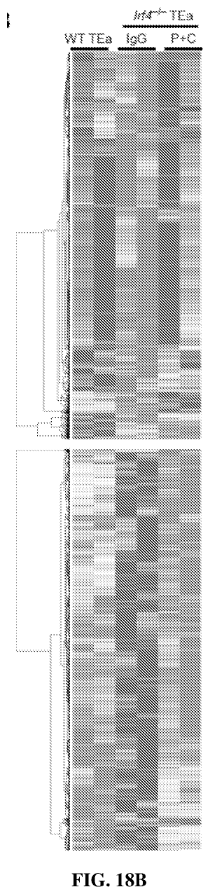

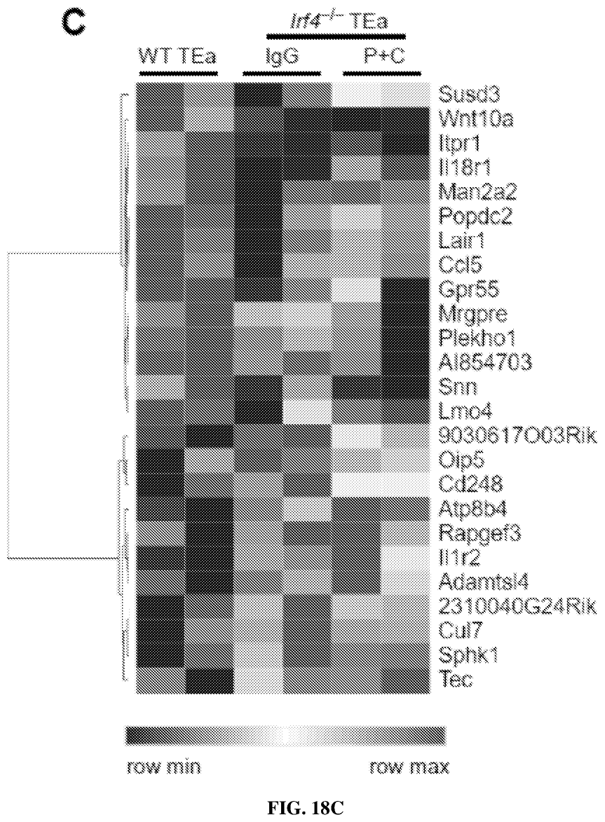

Identification of un-restored gene expression in Irf4-deficient alloreactive T cells upon immune checkpoint blockade. TCR-transgenic TEa CD4⁺ T cells (B6 background) recognize a Balb/c I-Ea allopeptide presented by B6 antigen presenting cells, and were used to assess the effects of immune checkpoint blockade on Irf4-deficient alloreactive T cells (8). Herein, the gene expression profiles between WT and Irf4^{-/-} TEa cells following heart transplantation and checkpoint blockade were compared. CD45.1⁺ B6 mice were adoptively transferred with either CD45.2⁺ WT TEa or CD45.2⁺ Irf4^{-/-} TEa cells on day −1, and transplanted with BALB/c hearts on day 0. Recipients transferred with Irf4^{-/-} TEa cells were further treated with rat IgG or anti-PD-L1 plus anti-CTLA-4 mAbs (P+C group) on days 0, 3, and 5. Adoptively transferred CD45.2⁺ TEa cells were isolated from splenocytes on day 6 by flow cytometry sorting (FIG. 18A). RNA was isolated and gene expression profiles were determined by microarray analysis. Differentially expressed genes between adoptively transferred WT TEa and Irf4^{-/-} TEa cells (IgG group) are shown in FIG. 18B. Importantly, checkpoint blockade (Irf4^{-/-} TEa; P+C group) restored the expression levels of the majority of WT TEa cell-expressed genes in Irf4^{-/-} TEa cells (FIG. 18B), which may explain why checkpoint blockade robustly reversed the initial dysfunction of Irf4^{-/-} T cells. Some of the unrestored genes in Irf4^{-/-} TEa cells following checkpoint blockade were shown in FIG. 18C.

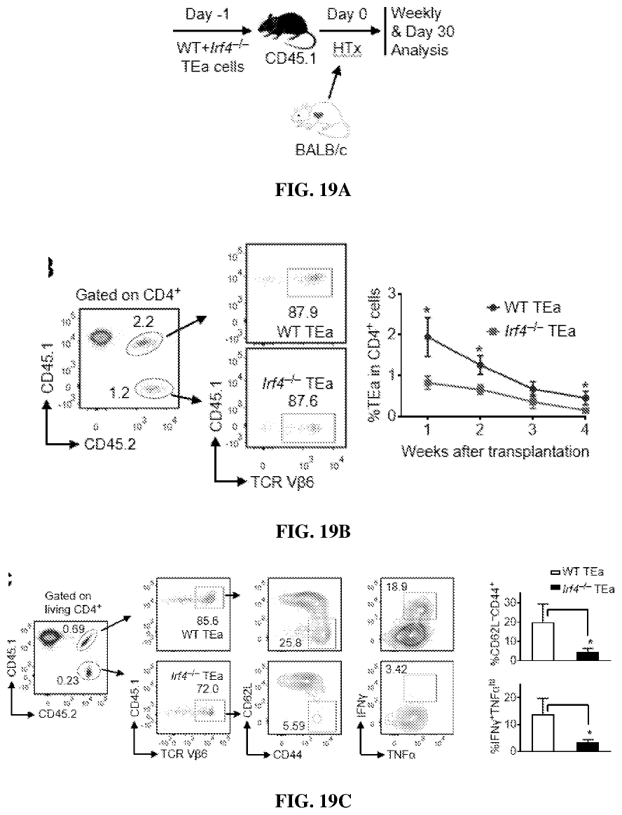

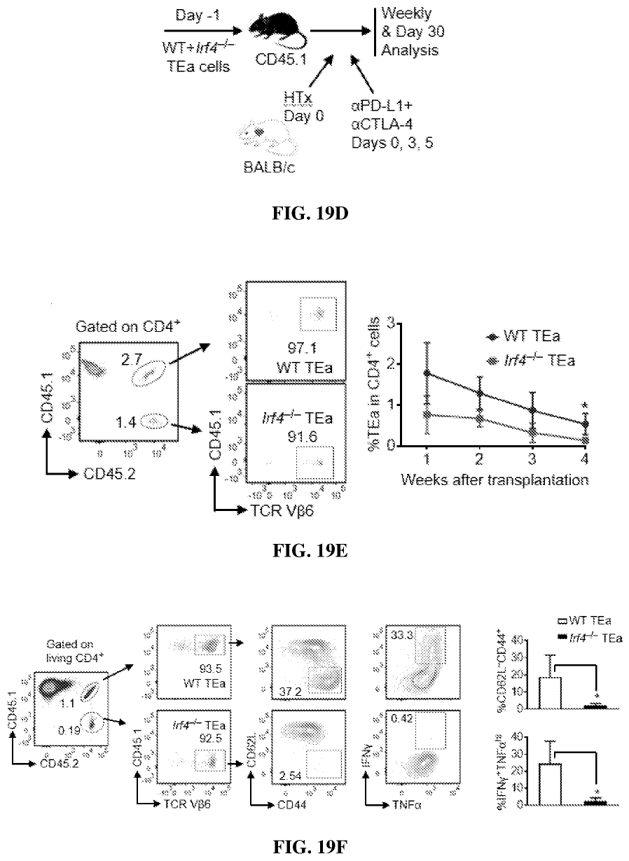

Checkpoint blockade does not restore effector memory cell generation from Irf4-deficient alloreactive T cells. To track the fate of Irf4^{-/-} alloreactive T cells, CD45.1⁺ B6 mice were adoptively transferred with mixed splenocytes containing a 1:1 ratio of CD45.1⁺ CD45.2⁺ WT TEa [from (TEa×CD45.1)F1 mice] and CD45.2⁺ Irf4^{-/-}˜ TEa cells (from Irf4^{-/-} TEa mice) one day prior to BALB/c heart transplantation (FIG. 19A). TEa cell frequencies in peripheral blood were assessed weekly post-grafting. Flow cytometry plots in FIG. 19B show the gating strategy detecting co-transferred CD45.1⁺ CD45.2⁺ TCR Vβ6⁺ WT TEa and CD45.1⁻ CD45.2⁺ TCR Vβ6⁺ Irf4^{-/-} TEa cells in peripheral blood at one week post-grafting. Both WT TEa and Irf4^{-/-} TEa cell frequencies were gradually decreased in peripheral blood (FIG. 19B, line graph). On day 30 post-grafting, splenocytes of transplant recipients were harvested and analyzed. The percentage of Irf4^{-/-} TEa cells among CD4⁺ splenocytes was significantly lower than that of WT TEa cells. Flow cytometry plots in FIG. 19C show the gating strategy detecting TEa cell populations, and the percentages of CD62L⁻CD44⁺ effector memory and IFN-γ⁺TNF-α^{hi} cells within WT TEa (top plots) and Irf4^{-/-} TEa (bottom plots) cell populations, respectively. Compared to WT TEa cells, Irf4^{-/-} TEa cells exhibited significantly lower frequencies of effector memory and IFN-γ/TNF-α producing cells (FIG. 19C, bar graphs). Percentages of IL-2⁺, PD-1⁺, and CX3CR1⁺, as well as mean fluorescence intensity (MFI) of CCR2 and CCR7 were not significantly different between WT and Irf4^{-/-} TEa cells in spleens when n=3 per group.

To track the fate of Irf4^{-/-} alloreactive T cells following checkpoint blockade, CD45.1⁺ B6 mice received cell transfer and heart transplantation as mentioned above, and treated with anti-PD-L1 plus anti-CTLA-4 mAbs on days 0, 3, and 5 post-grafting (FIG. 19D). Both WT TEa and Irf4^{-/-} TEa cell frequencies remained gradually declined in peripheral blood despite of checkpoint blockade (FIG. 19E). On day 30 post-grafting, the percentage of Irf4^{-/-} TEa cells among CD4⁺ splenocytes was significantly lower than that of WT TEa cells. Irf4^{-/-} TEa cells displayed significantly lower frequencies of effector memory and IFN-γ/TNF-α producing cells than those of WT TEa cells (FIG. 19F). Percentages of IL-2⁺, perforin/granzyme B⁺, PD-1⁺, and CX3CR1⁺, as well as MFI of CCR2, CCR7, TIM-3, and LAG-3 were not significantly different between WT and Irf4^{-/-} TEa cells in spleens when n=3 per group. Of note, CX3CR1 has been reported as a marker for anti-PD-1 therapy-responsive T cells, whereas CCR2 expressed on T cells has been shown to modulate the effector/regulatory T cell ratio. Taken together, checkpoint blockade does not restore cell frequency, effector memory cell generation, and IFN-γ/TNF-α production of Irf4^{-/-} TEa cells at day 30 post-grafting.

### Discussion

Alloreactive T cells are central to transplant outcomes (rejection versus tolerance), but how their functional fates are regulated transcriptionally remains unclear. IRF4 is a key transcriptional switch controlling the functional versus dysfunctional fate decision of alloreactive CD4⁺ T cells. Example 1 shows that reduction of IRF4 induced heart transplant acceptance by driving allogeneic CD4⁺ T cell dysfunction (8). In this example, it is further demonstrated that robust transplant tolerance was established following heart acceptance in Irf4^{fl/fl}Cd4-Cre mice, characterized by the subsequent acceptance of donor-type but not third-party skin allografts. More strikingly, immune checkpoint blockade during initial days post heart grating restored acute heart allograft rejection, but did not break the later establishment of transplant tolerance in Irf4^{fl/fl}Cd4-Cre recipients. By tracking alloreactive CD4⁺ T cells in heart-transplant recipients, it was found that checkpoint blockade restored the expression levels of the majority of WT T cell-expressed genes in Irf4^{-/-} T cells. Un-restored genes following checkpoint blockade may be responsible for the reinvigorated Irf4^{-/-} T cells to become re-dysfunction.

Experimental transplant tolerance was originally achieved 65 years ago. Medawar and colleagues demonstrated that mice injected at birth with allogeneic cells were subsequently able to accept skin allografts from the same donor strain (13). Since then, transplant tolerance was further achieved in adult animals through various approaches, including co-stimulatory blockade and induction of hematopoietic chimerism (14, 15). Deletion, anergy and Treg suppression of alloreactive T cells contribute to the induction and maintenance of transplant tolerance (16-19). Example 1 shows that ablation of a single transcription factor, IRF4, in T cells induced heart allograft acceptance. Mechanistically, IRF4 deletion did not reduce the total number of T cells in mice, but rather directed activated CD4⁺ T cells into a dysfunctional cell fate, which was associated with reduced production of effector cytokines, impaired cell expansion, and elevated expression of PD-1, Helios, and other negative regulators of T cell function (8). Herein, it is demonstrated that ablation of IRF4 in T cells induced robust transplant tolerance in heart allograft recipients, resulting in the acceptance of secondary donor-type skin allografts. Irf4^{fl/fl}Cd4-Cre mice were capable of rejecting primary skin allografts and secondary third-party skin allografts. It is possible that heart and skin allografts exhibit different capabilities in driving the dysfunctional differentiation of Irf4-deficient T cells. Induction of T cell dysfunction by targeting IRF4 represents a novel approach for achieving transplant tolerance.

Transient checkpoint blockade during the initial days post heart grafting rescued the anti-allograft function of Irf4-deficient T cells by restoring their ability to undergo proliferation and IFN-γ production. However, checkpoint blockade starting from day 30 post heart grafting did not restore allograft rejection in Irf4^{fl/fl}Cd4-Cre recipients (8). Thus, a progressive process may exist to establish the irreversible dysfunctional state of Irf4-deficient T cells in heart graft recipients. In this study, the influence of initial checkpoint blockade-mediated heart allograft rejection on the survival of subsequently transplanted skin allografts was investigated. Strikingly, checkpoint blockade induced acute heart transplant rejection in Irf4^{fl/fl}Cd4-Cre recipients, but did not prevent the later establishment of transplant tolerance. Hence, similar to the re-exhaustion of reinvigorated T cells in a chronic infection model (20), reinvigorated Irf4-deficient T cells also became re-dysfunction in this model. By comparing between WT and Irf4^{-/-} alloreactive CD4⁺ T cells in heart graft recipients, un-restored gene expression in Irf4^{-/-} T cells upon immune checkpoint blockade was identified.

It is a challenge to determine whether transplant tolerance has been achieved in the clinic, and how to proceed if it has not. Successful weaning from immunosuppression can be an indication of transplant tolerance, but it remains unclear how to stratify transplant recipients according to their likelihood of being able to discontinue immunosuppression. Even episodes of acute rejection did not preclude successful immunosuppressive drug withdrawal in some patients (21). Hence, there is a fundamental need to reveal the determinants of T cell fate in transplant tolerance. Herein, the inventors identified IRF4 as a key determinant governing transplant tolerance, which will advance the understanding and further characterization of tolerogenic T cell fate. Moreover, the MEK1/2 inhibitor Trametinib inhibited IRF4 expression in activated T cells and prolonged heart allograft survival in WT mice (8), which shows successful pharmacological inhibition of IRF4 (22).

### REFERENCES

1. Sawinski D, Trofe-Clark J, Leas B, et al. Calcineurin Inhibitor Minimization, Conversion, Withdrawal, and Avoidance Strategies in Renal Transplantation: A Systematic Review and Meta-Analysis. Am J Transplant. 2016; 16(7):2117-2138.
2. Li X C, Rothstein D M, Sayegh M H. Costimulatory pathways in transplantation: challenges and new developments. Immunol Rev. 2009; 229(1):271-293.
3. Miyahara Y, Khattar M, Schroder P M, et al. Anti-TCRbeta mAh induces long-term allograft survival by reducing antigen-reactive T cells and sparing regulatory T cells. Am J Transplant. 2012; 12(6):1409-1418.
4. Chen W, Ghobrial R M, Li X C. The Evolving Roles of Memory Immune Cells in Transplantation. Transplantation. 2015; 99(10):2029-2037.
5. Ochiai K, Maienschein-Cline M, Simonetti G, et al. Transcriptional regulation of germinal center B and plasma cell fates by dynamical control of IRF4. Immunity. 2013; 38(5):918-929.
6. Huber M, Lohoff M. IRF4 at the crossroads of effector T-cell fate decision. Eur J Immunol. 2014; 44(7): 1886-1895.
7. Vander Lugt B, Khan A A, Hackney J A, et al. Transcriptional programming of dendritic cells for enhanced MHC class II antigen presentation. Nat Immunol 2014; 15(2):161-167.
8. Wu J, Zhang H, Shi X, et al. Ablation of Transcription Factor IRF4 Promotes Transplant Acceptance by Driving Allogenic CD4(+) T Cell Dysfunction. Immunity. 2017; 47(6): 1114-1128 e6.
9. Alhamad T, Venkatachalam K, Linette G P, Brennan D C. Checkpoint Inhibitors in Kidney Transplant Recipients and the Potential Risk of Rejection. Am J Transplant. 2016; 16(4): 1332-1333.
10. Lipson E J, Bagnasco S M, Moore J, et al. Tumor Regression and Allograft Rejection after Administration of Anti-PD-1. N Engl J Med. 2016; 374(9):896-898.
11. Philip M, Fairchild L, Sun L, et al. Chromatin states define tumour-specific T cell dysfunction and reprogramming. Nature. 2017; 545(7655):452-456.
12. Garrod K R, Cahalan M D. Murine skin transplantation. J Vis Exp. 2008(11).
13. Billingham R E, Brent L, Medawar P B. Actively acquired tolerance of foreign cells. Nature. 1953; 172(4379):603-606.
14. Larsen C P, Elwood E T, Alexander D Z, et al. Long-term acceptance of skin and cardiac allografts after blocking CD40 and CD28 pathways. Nature. 1996; 381(6581):434-438.
15. Wekerle T, Kurtz J, Ito H, et al. Allogeneic bone marrow transplantation with co-stimulatory blockade induces macrochimerism and tolerance without cytoreductive host treatment. Nat Med 2000; 6(4):464-469.
16. Monk N J, Hargreaves R E, Marsh J E, et al. Fc-dependent depletion of activated T cells occurs through CD40L-specific antibody rather than costimulation blockade. Nat Med 2003; 9(10): 1275-1280.
17. Daley S R, Cobbold S P, Waldmann H. Fc-disabled anti-mouse CD40L antibodies retain efficacy in promoting transplantation tolerance. Am J Transplant. 2008; 8(11):2265-2271.
18. Taylor P A, Noelle R J, Blazar B R. CD4(+)CD25(+) immune regulatory cells are required for induction of tolerance to alloantigen via costimulatory blockade. J Exp Med 2001; 193(11): 1311-1318.
19. Zuber J, Sykes M. Mechanisms of Mixed Chimerism-Based Transplant Tolerance. Trends Immunol. 2017; 38(11):829-843.
20. Pauken K E, Sammons M A, Odorizzi P M, et al. Epigenetic stability of exhausted T cells limits durability of reinvigoration by PD-1 blockade. Science. 2016; 354(6316): 1160-1165.
21. Miller M L, Alegre M L, Chong A S. Transplantation tolerance after allograft rejection. Curr Opin Organ Transplant. 2017; 22(1):64-70.
22. Chennupati V, Held W. Feeling Exhausted? Tuning Irf4 Energizes Dysfunctional T Cells. Immunity. 2017; 47(6):1009-1011.

SEQUENCES

SEQ ID NO: 1. Human IRF4 amino acid sequence
(isoform 1).
MNLEGGGRGGEFGMSAVSCGNGKLRQWLIDQIDSGKYPGLVWENEEKSI
FRIPWKHAGKQDYNREEDAALFKAWALFKGKFREGIDKPDPPTWKTRLR
CALNKSNDFEELVERSQLDISDPYKVYRIVPEGAKKGAKQLTLEDPQMS
MSHPYTMTTPYPSLPAQQVHNYMMPPLDRSWRDYVPDQPHPEIPYQCPM
TFGPRGHHWQGPACENGCQVTGTFYACAPPESQAPGVPTEPSIRSAEAL
AFSDCRLHICLYYREILVKELTTSSPEGCRISHGHTYDASNLDQVLFPY
PEDNGQRKNIEKLLSHLERGVVLWMAPDGLYAKRLCQSRIYWDGPLALC
NDRPNKLERDQTCKLFDTQQFLSELQAFAHHGRSLPRFQVTLCFGEEFP
DPQRQRKLITAHVEPLLARQLYYFAQQNSGHFLRGYDLPEHISNPEDYH
RSIRHSSIQE
SEQ ID NO: 2. Human IRF4 nucleic acid sequence
(isoform 1).
ATGAACCTGGAGGGCGGCGGCCGAGGCGGAGAGTTCGGCATGAGCGCGG
TGAGCTGCGGCAACGGGAAGCTCCGCCAGTGGCTGATCGACCAGATCGA
CAGCGGCAAGTACCCCGGGCTGGTGTGGGAGAACGAGGAGAAGAGCATC
TTCCGCATCCCCTGGAAGCACGCGGGCAAGCAGGACTACAACCGCGAGG
AGGACGCCGCGCTCTTCAAGGCTTGGGCACTGTTTAAAGGAAAGTTCCG
AGAAGGCATCGACAAGCCGGACCCTCCCACCTGGAAGACGCGCCTGCGG
TGCGCTTTGAACAAGAGCAATGACTTTGAGGAACTGGTTGAGCGGAGCC
AGCTGGACATCTCAGACCCGTACAAAGTGTACAGGATTGTTCCTGAGGG
AGCCAAAAAAGGAGCCAAGCAGCTCACCCTGGAGGACCCGCAGATGTCC
ATGAGCCACCCCTACACCATGACAACGCCTTACCCTTCGCTCCCAGCCC
AGCAGGTTCACAACTACATGATGCCACCCCTCGACCGAAGCTGGAGGGA
CTACGTCCCGGATCAGCCACACCCGGAAATCCCGTACCAATGTCCCATG
ACGTTTGGACCCCGCGGCCACCACTGGCAAGGCCCAGCTTGTGAAAATG
GTTGCCAGGTGACAGGAACCTTTTATGCTTGTGCCCCACCTGAGTCCCA
GGCTCCCGGAGTCCCCACAGAGCCAAGCATAAGGTCTGCCGAAGCCTTG
GCGTTCTCAGACTGCCGGCTGCACATCTGCCTGTACTACCGGGAAATCC
TCGTGAAGGAGCTGACCACGTCCAGCCCCGAGGGCTGCCGGATCTCCCA
TGGACATACGTATGACGCCAGCAACCTGGACCAGGTCCTGTTCCCCTAC
CCAGAGGACAATGGCCAGAGGAAAAACATTGAGAAGCTGCTGAGCCACC
TGGAGAGGGGCGTGGTCCTCTGGATGGCCCCCGACGGGCTCTATGCGAA
AAGACTGTGCCAGAGCAGGATCTACTGGGACGGGCCCCTGGCGCTGTGC
AACGACCGGCCCAACAAACTGGAGAGAGACCAGACCTGCAAGCTCTTTG
ACACACAGCAGTTCTTGTCAGAGCTGCAAGCGTTTGCTCACCACGGCCG
CTCCCTGCCAAGATTCCAGGTGACTCTATGCTTTGGAGAGGAGTTTCCA
GACCCTCAGAGGCAAAGAAAGCTCATCACAGCTCACGTAGAACCTCTGC
TAGCCAGACAACTATATTATTTTGCTCAACAAAACAGTGGACATTTCCT
GAGGGGCTACGATTTACCAGAACACATCAGCAATCCAGAAGATTACCAC
AGATCTATCCGCCATTCCTCTATTCAAGAATGA
SEQ ID NO: 3. A 2 kb promoter sequence upstream
of Pdcd1 in mouse. Putative IRF4 binding sites
are shown in bold.
GAGGCTGTGTGGGTTCTATCATGTGTATTGGGGATTGCGTGTGTGTTAA
GAGATAGCATTACGTAGGACTCTATTGGGGAATAGAGTGTGTTAGAGAT
CTCATATCTGTATCTGAAGATAGCATGAGCCCTGAGGATTGTGTGTGTT
TATTGCAGGATAGTATATGTGTTTGAGATTGTGTGGGCTCTATCATGTG
TGCTGGGGATTGCATGTGTGTGGCTATTGGCATAGGGAAAGTAATTAGG
GCTACCCCAGCAACAGCCTTGCTCCTCACCACACTGCTAGGGAGGAAAG
GAGAAAGTAAGGACCAGAAGAAGGTACAGCAGAAGGGGAAAAGAGAAAG
ATAGGCAGAGGTCCTTTCACTCTCCACGGAAAGATATTCCAGTACCTGC
AGGCCTGAGGTGTTGTGGAAAGCAATAGGGATGTGCTGACAGCCTGCTG
CTCGGGCTCCTGCTCCTCTCCAGTCCTCATGATCTGCTCTGTATAGTCT
GAGTCCCATAGGAAGATTCTAGATAGAACATCAGAGCAAGGCAGGGTGG
AATCTGTCCAGAATTCACAATAGGCTCATAGCCAGTATCTAATCACCTA
GCCTTGACAGGGGTCTTGCTACCCTGAGCATGCCAGAAAGACATAAAGG
TATAAAGGAGGCTCTGTAACAGCCAGGCGTGGGGAGGGGATCCCCCTAG
CTTCTGCCCACAGGCCCCATGCTGAGACTGGAGGCGGCCAGTCTGTGCC
TCACACTCTTTTTCCATTTCTGTGCTGTTCAAAGTAATGTTTCCTTCCC
CACCAAGCTAGTGCCTCTGAACCTGGGTGGCTGAGGCAGTTGCCAGATG
GTTTCCAGGCGGGCTGCCTATTTTAGGGTGGTGAGACCCACACATCTCA
TTGCTAATATTAGCAGTTTCGTTTTCCCTTTTTTTCCCATTCACTGTGG
CGAAACACAGAGAGCAGAATGATTAAATCATCAGAATGCCCCAGAAATG
ACTAGCCAGCCAGGTACTATGCATGCACACAAGTCGGCCCACCCCACCT
AATCCCAGAGAGACAAGCAGGAGGTGAGGTGGGCCTCCACCTCCTAGGG
ACTGAGGAAAGTTGACTGGGAAAGACCTAGAAATTGAGTCTACCCCAGC
CTGGTGTTAGGTTTTTCTCAGGGGAAGAGAAAGATGCAGGGCAGCAGAG
CTAGCAAACCTAAGACAACTATAGAAGCAGAGAAAACAGTGAGATCCGG
GCAGCAGATCCAGCATCTTGAAAGGAAGAAAAGCCTTAAGAGAAAGCAA
GACCAGGCCCAGGGTCTTTCTGAACCTACAGGGGTGTCTGGAGAGGAAA
GGCATCGTCTCGGGTCCTAGGAAATGTTCACTATAGCCCTTCGAGGCCT
CCTCTGACCCATCAAACGGGAGCATGTGGGATAGCTGGGCTCTTGCTCC
TCAGTAGTAAAGGACTAAGGCATAGCTCAGGGCATTCAAGGCCACGCAT
GGCAGACAAGGTAGGGGAGGGTCCAGCTTGCCCTCGCTGCGGCCATAGG
TACCAAAGCCAGGCCTCGACACCCACCCTCCAAAGGGACAAGAGTCTGG
CCCTAGTTTCAGTCTCTCTCAGCCCTGGGAGCTAAGGCTCGATCGGGGT
ACCAGGAATGGAAAAGACCAAACCTACCCACAAGAGGGGCTAGAAATGG
AGAGGACCCCATAGCAGGACAAGAGGCAAGGACAGCTAGTCAGAGAGAA
CCCCCCCTCTCTGCTCCCCAATCTCTCACTAGTCCCTTACCTGCTCCTC
CCAGGCATCGTTCCCTCCCACTCCCCTCCCCCTTCCATGCCCCTCCCCC
ACCTCTAGTTGCCTGTTCTCCCACCCTTGTGGAGGTGGAGGAAGAGGGG
GCGGGAGCCAAGAACAGGTCTCCTCCCTCCAACATGACCTGGGACAGTT
TCCTTTCCGCTACAGACAACTCTGCCTGAGCAGCGGGGAGGAGGAAGAG
GAGACTGCTACTGAAGGCGACACTGCCAGGGGCTCTGGGCATGTGGGTC
CGGCAGGTACCCTGG
SEQ ID NO: 4. A CHIP5 forward primer.
AGGACCAGAAGAAGGTACAGCA
SEQ ID NO: 5. A CHIP5 reverse primer.
TTGCTTTCCACAACACCTCAGG
SEQ ID NO: 6. A CHIP4 forward primer.
AGACCCACACATCTCATTGCTAAT
SEQ ID NO: 7. A CHIP4 reverse primer.
CCTGGCTGGCTAGTCATTTCTG
SEQ ID NO: 8. A CHIP3 forward primer.
GCAGCAGATCCAGCATCTTGAA
SEQ ID NO: 9. A CHIP3 reverse primer.
TGCCTTTCCTCTCCAGACACC
SEQ ID NO: 10. A CHIP2 forward primer.
CCACCCTCCAAAGGGACAAGA
SEQ ID NO: 11. A CHIP2 reverse primer.
CCCTCTTGTGGGTAGGTTTGGT
SEQ ID NO: 12. A CHIP1 forward primer.
CAAGAACAGGTCTCCTCCCTCCAA
SEQ ID NO: 13. A CHIP1 reverse primer.
AGTGTCGCCTTCAGTAGCAGTCT
SEQ ID NO: 14. A binding site in an Ikzf2 intron.
A putative IRF4 binding sites is shown in bold.
CATACTCTTAACAGTCAGAATGAAACCTGGGTGG
SEQ ID NO: 15. A control forward primer.
GCGCAAGGTAAGAACTGTCC
SEQ ID NO: 16. A control reverse primer.
ACAGCCCTCCAAATGTCAAC
SEQ ID NO: 17. A -3.7 forward primer.
AGGGTGAGCAGGCGAGAGCAA
SEQ ID NO: 18. A -3.7 reverse primer.
AGCACAGGGAAGAAGCTGTTGGG
SEQ ID NO: 19. A CR-C forward primer.
CCTCACCTCCTGCTTGTCTCTC
SEQ ID NO: 20. A CR-C reverse primer.
GTGAGACCCACACATCTCATTGC
SEQ ID NO: 21. A CR-B forward primer.
CTCTGACTAGCTGTCCTTGCCTC
SEQ ID NO: 22. A CR-B reverse primer.
CTCGACACCCACCCTCCAAAG
SEQ ID NO: 23. A
GGAGGGGATAGGCGCTGGGT
SEQ ID NO: 24. A
TCTGGGCCAAGCCATCCGGT
SEQ ID NO: 25. Human IRF4 nucleic acid sequence
(isoform 1) with 5′ and 3′ UTR sequences.
ACCTCGCACTCTCAGTTTCACCGCTCGATCTTGGGACCCACCGCTGCCC
TCAGCTCCGAGTCCAGGGCGAGTGCAGAGCAGAGCGGGCGGAGGACCCC
GGGCGCGGGCGCGGACGGCACGCGGGCATGAACCTGGAGGGCGGCGGCC
GAGGCGGAGAGTTCGGCATGAGCGCGGTGAGCTGCGGCAACGGGAAGCT
CCGCCAGTGGCTGATCGACCAGATCGACAGCGGCAAGTACCCCGGGCTG
GTGTGGGAGAACGAGGAGAAGAGCATCTTCCGCATCCCCTGGAAGCACG
CGGGCAAGCAGGACTACAACCGCGAGGAGGACGCCGCGCTCTTCAAGGC
TTGGGCACTGTTTAAAGGAAAGTTCCGAGAAGGCATCGACAAGCCGGAC
CCTCCCACCTGGAAGACGCGCCTGCGGTGCGCTTTGAACAAGAGCAATG
ACTTTGAGGAACTGGTTGAGCGGAGCCAGCTGGACATCTCAGACCCGTA
CAAAGTGTACAGGATTGTTCCTGAGGGAGCCAAAAAAGGAGCCAAGCAG
CTCACCCTGGAGGACCCGCAGATGTCCATGAGCCACCCCTACACCATGA
CAACGCCTTACCCTTCGCTCCCAGCCCAGCAGGTTCACAACTACATGAT
GCCACCCCTCGACCGAAGCTGGAGGGACTACGTCCCGGATCAGCCACAC
CCGGAAATCCCGTACCAATGTCCCATGACGTTTGGACCCCGCGGCCACC
ACTGGCAAGGCCCAGCTTGTGAAAATGGTTGCCAGGTGACAGGAACCTT
TTATGCTTGTGCCCCACCTGAGTCCCAGGCTCCCGGAGTCCCCACAGAG
CCAAGCATAAGGTCTGCCGAAGCCTTGGCGTTCTCAGACTGCCGGCTGC
ACATCTGCCTGTACTACCGGGAAATCCTCGTGAAGGAGCTGACCACGTC
CAGCCCCGAGGGCTGCCGGATCTCCCATGGACATACGTATGACGCCAGC
AACCTGGACCAGGTCCTGTTCCCCTACCCAGAGGACAATGGCCAGAGGA
AAAACATTGAGAAGCTGCTGAGCCACCTGGAGAGGGGCGTGGTCCTCTG
GATGGCCCCCGACGGGCTCTATGCGAAAAGACTGTGCCAGAGCAGGATC
TACTGGGACGGGCCCCTGGCGCTGTGCAACGACCGGCCCAACAAACTGG
AGAGAGACCAGACCTGCAAGCTCTTTGACACACAGCAGTTCTTGTCAGA
GCTGCAAGCGTTTGCTCACCACGGCCGCTCCCTGCCAAGATTCCAGGTG
ACTCTATGCTTTGGAGAGGAGTTTCCAGACCCTCAGAGGCAAAGAAAGC
TCATCACAGCTCACGTAGAACCTCTGCTAGCCAGACAACTATATTATTT
TGCTCAACAAAACAGTGGACATTTCCTGAGGGGCTACGATTTACCAGAA
CACATCAGCAATCCAGAAGATTACCACAGATCTATCCGCCATTCCTCTA
TTCAAGAATGAAAAATGTCAAGATGAGTGGTTTTCTTTTTCCTTTTTTT
TTTTTTTTTTTGATACGGGGATACGGGGTCTTGCTCTGTCTCCCAGGCT
GGAGTGCAGTGACACAATCTCAGCTCACTGTGACCTCCGCCTCCTGGGT
TCAAGAGACTCTCCTGCCTCAGCCTCCCTGGTAGCTGGGATTACAGGTG
TGAGCCACTGCACCCACCCAAGACAAGTGATTTTCATTGTAAATATTTG
ACTTTAGTGAAAGCGTCCAATTGACTGCCCTCTTACTGTTTTGAGGAAC
TCAGAAGTGGAGATTTCAGTTCAGCGGTTGAGGAGAATTGCGGCGAGAC
AAGCATGGAAAATCAGTGACATCTGATTGGCAGATGAGCTTATTTCAAA
AGGAAGGGTGGCTTTGCATTTCTTGTGTTCTATAGACTGCCATCATTGA
TGATCACTGTGAAAATTGACCAAGTGATGTGTTTACATTTACTGAAATG
TGCTCTTTAATTTGTTGTAGATTAGGTCTTGCTGGAAGACAGAGAAAAC
TTGCCTTTCAGTATTGACACTGACTAGAGTGATGACTGCTTGTAGGTAT
GTCTGTGCCATTTCTCAGGGAAGTAAGATGTAAATTGAAGAAGCCTCAC
ACGTAAAAGAAATGTATTAATGTATGTAGGAGCTGCAGTTCTTGTGGAA
GACACTTGCTGAGTGAAGGAAATGAATCTTTGACTGAAGCCGTGCCTGT
AGCCTTGGGGAGGCCCATCCCCCACCTGCCAGCGGTTTCCTGGTGTGGG
TCCCTCTGCCCCACCCTCCTTCCCATTGGCTTTCTCTCCTTGGCCTTTC
CTGGAAGCCAGTTAGTAAACTTCCTATTTTCTTGAGTCAAAAAACATGA
GCGCTACTCTTGGATGGGACATTTTTGTCTGTCCTACAATCTAGTAATG
TCTAAGTAATGGTTAAGTTTTCTTGTTTCTGCATCTTTTTGACCCTCAT
TCTTTAGAGATGCTAAAATTCTTCGCATAAAGAAGAAGAAATTAAGGAA
CATAAATCTTAATACTTGAACTGTTGCCCTTCTGTCCAAGTACTTAACT
ATCTGTTCCCTTCCTCTGTGCCACGCTCCTCTGTTTGCTTGGCTGTCCA
GCGATCAGCCATGGCGACACTAAAGGAGGAGGAGCCGGGGACTCCCAGG
CTGGAGAGCACTGCCAGGACCCACCACTGGAAGCAGGATGGAGCTGACT
ACGGAACTGCACACTCAGTGGGCTGTTTCTGCTTATTTCATCTGTTCTA
TGCTTCCTCGTGCCAATTATAGTTTGACAGGGCCTTAAAATTACTTGGC
TTTTTCCAAATGCTTCTATTTATAGAATCCCAAAGACCTCCACTTGCTT
AAGTATACCTATCACTTACATTTTTGTGGTTTTGAGAAAGTACAGCAGT
AGACTGGGGCGTCACCTCCAGGCCGTTTCTCATACTACAGGATATTTAC
TATTACTCCCAGGATCAGCAGAAGATTGCGTAGCTCTCAAATGTGTGTT
CCTGCTTTTCTAATGGATATTTTAAATTCATTCAACAAGCACCTAGTAA
GTGCCTGCTGTATCCCTACATTACACAGTTCAGCCTTTATCAAGCTTAG
TGAGCAGTGAGCACTGAAACATTATTTTTTAATGTTTAAAAAGTTTCTA
ATATTAAAGTCAGAATATTAATACAATTAATATTAATATTAACTACAGA
AAAGACAAACAGTAGAGAACAGCAAAAAAATAAAAAGGATCTCCTTTTT
TCCCAGCCCAAATTCTCCTCTCTAAAAGTGTCCACAAGAAGGGGTGTTT
ATTCTTCCAACACATTTCACTTTTCTGTAAATATACATAAACTTAAAAA
GAAAACCTCATGGAGTCATCTTGCACACACTTTCATGCAGTGCTCTTTG
TAGCTAACAGTGAAGATTTACCTCGTTCTGCTCAGAGGCCTTGCTGTGG
AGCTCCACTGCCATGTACCCAGTAGGGTTTGACATTTCATTAGCCATGC
AACATGGATATGTATTGGGCAGCAGACTGTGTTTCGTGAACTGCAGTGA
TGTATACATCTTATAGATGCAAAGTATTTTGGGGTATATTATCCTAAGG
GAAGATAAAGATGATATTAAGAACTGCTGTTTCACGGGGCCCTTACCTG
TGACCCTCTTTGCTGAAGAATATTTAACCCCACACAGCACTTCAAAGAA
GCTGTCTTGGAAGTCTGTCTCAGGAGCACCCTGTCTTCTTAATTCTCCA
AGCGGATGCTCCATTTCAATTGCTTTGTGACTTCTTCTTCTTTGTTTTT
TTAAATATTATGCTGCTTTAACAGTGGAGCTGAATTTTCTGGAAAATGC
TTCTTGGCTGGGGCCACTACCTCCTTTCCTATCTTTACATCTATGTGTA
TGTTGACTTTTTAAAATTCTGAGTGATCCAGGGTATGACCTAGGGAATG
AACTAGCTATGAAATACTCAGGGTTAGGAATCCTAGCACTTGTCTCAGG
ACTCTGAAAAGGAACGGCTTCCTCATTCCTTGTCTTGATAAAGTGGAAT
TGGCAAACTAGAATTTAGTTTGTACTCAGTGGACAGTGCTGTTGAAGAT
TTGAGGACTTGTTAAAGAGCACTGGGTCATATGGAAAAAATGTATGTGT
CTCCCAGGTGCATTTCTTGGTTTATGTCTTGTTCTTGAGATTTTGTATA
TTTAGGAAAACCTCAAGCAGTAATTAATATCTCCTGGAACACTATAGAG
AACCAAGTGACCGACTCATTTACAACTGAAACCTAGGAAGCCCCTGAGT
CCTGAGCGAAAACAGGAGAGTTAGTCGCCCTACAGGAAACCCAGCTAGA
CTATTGGGTATGAACTAAAAAGAGACTGTGCCATGGTGAGAAAAATGTA
AAATCCTACAGTGGAATGAGCAGCCCTTACAGTGTTGTTACCACCAAGG
GCAGGTAGGTATTAGTGTTTGAAAAAGCTGGTCTTTGAGCGAGGGCATA
AATACAGCTAGCCCCAGGGGTGGAACAACTGTGGGAGTCTTGGGTACTC
GCACCTCTTGGCTTTGTTGATGCTCCGCCAGGAAGGCCACTTGTGTGTG
CGTGTCAGTTACTTTTTTAGTAACAATTCAGATCCAGTGTAAACTTCCG
TTCATTGCTCTCCAGTCACATGCCCCCACTTCCCCACAGGTGAAAGTTT
TTCTGAAAGTGTTGGGATTGGTTAAGGTCTTTATTTGTATTACGTATCT
CCCCAAGTCCTCTGTGGCCAGCTGCATCTGTCTGAATGGTGCGTGAAGG
CTCTCAGACCTTACACACCATTTTGTAAGTTATGTTTTACATGCCCCGT
TTTTGAGACTGATCTCGATGCAGGTGGATCTCCTTGAGATCCTGATAGC
CTGTTACAGGAATGAAGTAAAGGTCAGTTTTTTTTGTATTGATTTTCAC
AGCTTTGAGGAACATGCATAAGAAATGTAGCTGAAGTAGAGGGGACGTG
AGAGAAGGGCCAGGCCGGCAGGCCAACCCTCCTCCAATGGAAATTCCCG
TGTTGCTTCAAACTGAGACAGATGGGACTTAACAGGCAATGGGGTCCAC
TTCCCCCTCTTCAGCATCCCCCGTACCCCACTTTTTGCTGAAAGAACTG
CCAGCAGGTAGGACCCCAGAGGCCCCCAAATGAAAGCTTGAATTTCCCC
TACTGGCTCTGCGTTTTGCTGAGATCTGTAGGAAAGGATGCTTCACAAA
CTGAGGTAGATAATGCTATGCTGTCGTTGGTATACATCATGAATTTTTA
TGTAAATTGCTCTGCAAAGCAAATTGATATGTTTGATAAATTTATGTTT
TTAGGTAAATAAAAACTTTTAAAAAGTTGTT
SEQ ID NO: 26. Human IRF4 amino acid sequence
(isoform 2).
MNLEGGGRGGEFGMSAVSCGNGKLRQWLIDQIDSGKYPGLVWENEEKSI
FRIPWKHAGKQDYNREEDAALFKAWALFKGKFREGIDKPDPPTWKTRLR
CALNKSNDFEELVERSQLDISDPYKVYRIVPEGAKKGAKQLTLEDPQMS
MSHPYTMTTPYPSLPAQVHNYMMPPLDRSWRDYVPDQPHPEIPYQCPMT
FGPRGHHWQGPACENGCQVTGTFYACAPPESQAPGVPTEPSIRSAEALA
FSDCRLHICLYYREILVKELTTSSPEGCRISHGHTYDASNLDQVLFPYP
EDNGQRKNIEKLLSHLERGVVLWMAPDGLYAKRLCQSRIYVVDGPLALC
NDRPNKLERDQTCKLFDTQQFLSELQAFAHHGRSLPRFQVTLCFGEEFP
DPQRQRKLITAHVEPLLARQLYYFAQQNSGHFLRGYDLPEHISNPEDYH
RSIRHSSIQE
SEQ ID NO: 27. Human IRF4 nucleic acid sequence
(isoform 2).
ATGAACCTGGAGGGCGGCGGCCGAGGCGGAGAGTTCGGCATGAGCGCGG
TGAGCTGCGGCAACGGGAAGCTCCGCCAGTGGCTGATCGACCAGATCGA
CAGCGGCAAGTACCCCGGGCTGGTGTGGGAGAACGAGGAGAAGAGCATC
TTCCGCATCCCCTGGAAGCACGCGGGCAAGCAGGACTACAACCGCGAGG
AGGACGCCGCGCTCTTCAAGGCTTGGGCACTGTTTAAAGGAAAGTTCCG
AGAAGGCATCGACAAGCCGGACCCTCCCACCTGGAAGACGCGCCTGCGG
TGCGCTTTGAACAAGAGCAATGACTTTGAGGAACTGGTTGAGCGGAGCC
AGCTGGACATCTCAGACCCGTACAAAGTGTACAGGATTGTTCCTGAGGG
AGCCAAAAAAGGAGCCAAGCAGCTCACCCTGGAGGACCCGCAGATGTCC
ATGAGCCACCCCTACACCATGACAACGCCTTACCCTTCGCTCCCAGCCC
AGGTTCACAACTACATGATGCCACCCCTCGACCGAAGCTGGAGGGACTA
CGTCCCGGATCAGCCACACCCGGAAATCCCGTACCAATGTCCCATGACG
TTTGGACCCCGCGGCCACCACTGGCAAGGCCCAGCTTGTGAAAATGGTT
GCCAGGTGACAGGAACCTTTTATGCTTGTGCCCCACCTGAGTCCCAGGC
TCCCGGAGTCCCCACAGAGCCAAGCATAAGGTCTGCCGAAGCCTTGGCG
TTCTCAGACTGCCGGCTGCACATCTGCCTGTACTACCGGGAAATCCTCG
TGAAGGAGCTGACCACGTCCAGCCCCGAGGGCTGCCGGATCTCCCATGG
ACATACGTATGACGCCAGCAACCTGGACCAGGTCCTGTTCCCCTACCCA
GAGGACAATGGCCAGAGGAAAAACATTGAGAAGCTGCTGAGCCACCTGG
AGAGGGGCGTGGTCCTCTGGATGGCCCCCGACGGGCTCTATGCGAAAAG
ACTGTGCCAGAGCAGGATCTACTGGGACGGGCCCCTGGCGCTGTGCAAC
GACCGGCCCAACAAACTGGAGAGAGACCAGACCTGCAAGCTCTTTGACA
CACAGCAGTTCTTGTCAGAGCTGCAAGCGTTTGCTCACCACGGCCGCTC
CCTGCCAAGATTCCAGGTGACTCTATGCTTTGGAGAGGAGTTTCCAGAC
CCTCAGAGGCAAAGAAAGCTCATCACAGCTCACGTAGAACCTCTGCTAG
CCAGACAACTATATTATTTTGCTCAACAAAACAGTGGACATTTCCTGAG
GGGCTACGATTTACCAGAACACATCAGCAATCCAGAAGATTACCACAGA
TCTATCCGCCATTCCTCTATTCAAGAATGA
SEQ ID NO: 28. Human IRF4 nucleic acid sequence
(isoform 2) with 5′ and 3′ UTR sequences.
ACCTCGCACTCTCAGTTTCACCGCTCGATCTTGGGACCCACCGCTGCCC
TCAGCTCCGAGTCCAGGGCGAGTGCAGAGCAGAGCGGGCGGAGGACCCC
GGGCGCGGGCGCGGACGGCACGCGGGCATGAACCTGGAGGGCGGCGGCC
GAGGCGGAGAGTTCGGCATGAGCGCGGTGAGCTGCGGCAACGGGAAGCT
CCGCCAGTGGCTGATCGACCAGATCGACAGCGGCAAGTACCCCGGGCTG
GTGTGGGAGAACGAGGAGAAGAGCATCTTCCGCATCCCCTGGAAGCACG
CGGGCAAGCAGGACTACAACCGCGAGGAGGACGCCGCGCTCTTCAAGGC
TTGGGCACTGTTTAAAGGAAAGTTCCGAGAAGGCATCGACAAGCCGGAC
CCTCCCACCTGGAAGACGCGCCTGCGGTGCGCTTTGAACAAGAGCAATG
ACTTTGAGGAACTGGTTGAGCGGAGCCAGCTGGACATCTCAGACCCGTA
CAAAGTGTACAGGATTGTTCCTGAGGGAGCCAAAAAAGGAGCCAAGCAG
CTCACCCTGGAGGACCCGCAGATGTCCATGAGCCACCCCTACACCATGA
CAACGCCTTACCCTTCGCTCCCAGCCCAGGTTCACAACTACATGATGCC
ACCCCTCGACCGAAGCTGGAGGGACTACGTCCCGGATCAGCCACACCCG
GAAATCCCGTACCAATGTCCCATGACGTTTGGACCCCGCGGCCACCACT
GGCAAGGCCCAGCTTGTGAAAATGGTTGCCAGGTGACAGGAACCTTTTA
TGCTTGTGCCCCACCTGAGTCCCAGGCTCCCGGAGTCCCCACAGAGCCA
AGCATAAGGTCTGCCGAAGCCTTGGCGTTCTCAGACTGCCGGCTGCACA
TCTGCCTGTACTACCGGGAAATCCTCGTGAAGGAGCTGACCACGTCCAG
CCCCGAGGGCTGCCGGATCTCCCATGGACATACGTATGACGCCAGCAAC
CTGGACCAGGTCCTGTTCCCCTACCCAGAGGACAATGGCCAGAGGAAAA
ACATTGAGAAGCTGCTGAGCCACCTGGAGAGGGGCGTGGTCCTCTGGAT
GGCCCCCGACGGGCTCTATGCGAAAAGACTGTGCCAGAGCAGGATCTAC
TGGGACGGGCCCCTGGCGCTGTGCAACGACCGGCCCAACAAACTGGAGA
GAGACCAGACCTGCAAGCTCTTTGACACACAGCAGTTCTTGTCAGAGCT
GCAAGCGTTTGCTCACCACGGCCGCTCCCTGCCAAGATTCCAGGTGACT
CTATGCTTTGGAGAGGAGTTTCCAGACCCTCAGAGGCAAAGAAAGCTCA
TCACAGCTCACGTAGAACCTCTGCTAGCCAGACAACTATATTATTTTGC
TCAACAAAACAGTGGACATTTCCTGAGGGGCTACGATTTACCAGAACAC
ATCAGCAATCCAGAAGATTACCACAGATCTATCCGCCATTCCTCTATTC
AAGAATGAAAAATGTCAAGATGAGTGGTTTTCTTTTTCCTTTTTTTTTT
TTTTTTTTGATACGGGGATACGGGGTCTTGCTCTGTCTCCCAGGCTGGA
GTGCAGTGACACAATCTCAGCTCACTGTGACCTCCGCCTCCTGGGTTCA
AGAGACTCTCCTGCCTCAGCCTCCCTGGTAGCTGGGATTACAGGTGTGA
GCCACTGCACCCACCCAAGACAAGTGATTTTCATTGTAAATATTTGACT
TTAGTGAAAGCGTCCAATTGACTGCCCTCTTACTGTTTTGAGGAACTCA
GAAGTGGAGATTTCAGTTCAGCGGTTGAGGAGAATTGCGGCGAGACAAG
CATGGAAAATCAGTGACATCTGATTGGCAGATGAGCTTATTTCAAAAGG
AAGGGTGGCTTTGCATTTCTTGTGTTCTATAGACTGCCATCATTGATGA
TCACTGTGAAAATTGACCAAGTGATGTGTTTACATTTACTGAAATGTGC
TCTTTAATTTGTTGTAGATTAGGTCTTGCTGGAAGACAGAGAAAACTTG
CCTTTCAGTATTGACACTGACTAGAGTGATGACTGCTTGTAGGTATGTC
TGTGCCATTTCTCAGGGAAGTAAGATGTAAATTGAAGAAGCCTCACACG
TAAAAGAAATGTATTAATGTATGTAGGAGCTGCAGTTCTTGTGGAAGAC
ACTTGCTGAGTGAAGGAAATGAATCTTTGACTGAAGCCGTGCCTGTAGC
CTTGGGGAGGCCCATCCCCCACCTGCCAGCGGTTTCCTGGTGTGGGTCC
CTCTGCCCCACCCTCCTTCCCATTGGCTTTCTCTCCTTGGCCTTTCCTG
GAAGCCAGTTAGTAAACTTCCTATTTTCTTGAGTCAAAAAACATGAGCG
CTACTCTTGGATGGGACATTTTTGTCTGTCCTACAATCTAGTAATGTCT
AAGTAATGGTTAAGTTTTCTTGTTTCTGCATCTTTTTGACCCTCATTCT
TTAGAGATGCTAAAATTCTTCGCATAAAGAAGAAGAAATTAAGGAACAT
AAATCTTAATACTTGAACTGTTGCCCTTCTGTCCAAGTACTTAACTATC
TGTTCCCTTCCTCTGTGCCACGCTCCTCTGTTTGCTTGGCTGTCCAGCG
ATCAGCCATGGCGACACTAAAGGAGGAGGAGCCGGGGACTCCCAGGCTG
GAGAGCACTGCCAGGACCCACCACTGGAAGCAGGATGGAGCTGACTACG
GAACTGCACACTCAGTGGGCTGTTTCTGCTTATTTCATCTGTTCTATGC
TTCCTCGTGCCAATTATAGTTTGACAGGGCCTTAAAATTACTTGGCTTT
TTCCAAATGCTTCTATTTATAGAATCCCAAAGACCTCCACTTGCTTAAG
TATACCTATCACTTACATTTTTGTGGTTTTGAGAAAGTACAGCAGTAGA
CTGGGGCGTCACCTCCAGGCCGTTTCTCATACTACAGGATATTTACTAT
TACTCCCAGGATCAGCAGAAGATTGCGTAGCTCTCAAATGTGTGTTCCT
GCTTTTCTAATGGATATTTTAAATTCATTCAACAAGCACCTAGTAAGTG
CCTGCTGTATCCCTACATTACACAGTTCAGCCTTTATCAAGCTTAGTGA
GCAGTGAGCACTGAAACATTATTTTTTAATGTTTAAAAAGTTTCTAATA
TTAAAGTCAGAATATTAATACAATTAATATTAATATTAACTACAGAAAA
GACAAACAGTAGAGAACAGCAAAAAAATAAAAAGGATCTCCTTTTTTCC
CAGCCCAAATTCTCCTCTCTAAAAGTGTCCACAAGAAGGGGTGTTTATT
CTTCCAACACATTTCACTTTTCTGTAAATATACATAAACTTAAAAAGAA
AACCTCATGGAGTCATCTTGCACACACTTTCATGCAGTGCTCTTTGTAG
CTAACAGTGAAGATTTACCTCGTTCTGCTCAGAGGCCTTGCTGTGGAGC
TCCACTGCCATGTACCCAGTAGGGTTTGACATTTCATTAGCCATGCAAC
ATGGATATGTATTGGGCAGCAGACTGTGTTTCGTGAACTGCAGTGATGT
ATACATCTTATAGATGCAAAGTATTTTGGGGTATATTATCCTAAGGGAA
GATAAAGATGATATTAAGAACTGCTGTTTCACGGGGCCCTTACCTGTGA
CCCTCTTTGCTGAAGAATATTTAACCCCACACAGCACTTCAAAGAAGCT
GTCTTGGAAGTCTGTCTCAGGAGCACCCTGTCTTCTTAATTCTCCAAGC
GGATGCTCCATTTCAATTGCTTTGTGACTTCTTCTTCTTTGTTTTTTTA
AATATTATGCTGCTTTAACAGTGGAGCTGAATTTTCTGGAAAATGCTTC
TTGGCTGGGGCCACTACCTCCTTTCCTATCTTTACATCTATGTGTATGT
TGACTTTTTAAAATTCTGAGTGATCCAGGGTATGACCTAGGGAATGAAC
TAGCTATGAAATACTCAGGGTTAGGAATCCTAGCACTTGTCTCAGGACT
CTGAAAAGGAACGGCTTCCTCATTCCTTGTCTTGATAAAGTGGAATTGG
CAAACTAGAATTTAGTTTGTACTCAGTGGACAGTGCTGTTGAAGATTTG
AGGACTTGTTAAAGAGCACTGGGTCATATGGAAAAAATGTATGTGTCTC
CCAGGTGCATTTCTTGGTTTATGTCTTGTTCTTGAGATTTTGTATATTT
AGGAAAACCTCAAGCAGTAATTAATATCTCCTGGAACACTATAGAGAAC
CAAGTGACCGACTCATTTACAACTGAAACCTAGGAAGCCCCTGAGTCCT
GAGCGAAAACAGGAGAGTTAGTCGCCCTACAGGAAACCCAGCTAGACTA
TTGGGTATGAACTAAAAAGAGACTGTGCCATGGTGAGAAAAATGTAAAA
TCCTACAGTGGAATGAGCAGCCCTTACAGTGTTGTTACCACCAAGGGCA
GGTAGGTATTAGTGTTTGAAAAAGCTGGTCTTTGAGCGAGGGCATAAAT
ACAGCTAGCCCCAGGGGTGGAACAACTGTGGGAGTCTTGGGTACTCGCA
CCTCTTGGCTTTGTTGATGCTCCGCCAGGAAGGCCACTTGTGTGTGCGT
GTCAGTTACTTTTTTAGTAACAATTCAGATCCAGTGTAAACTTCCGTTC
ATTGCTCTCCAGTCACATGCCCCCACTTCCCCACAGGTGAAAGTTTTTC
TGAAAGTGTTGGGATTGGTTAAGGTCTTTATTTGTATTACGTATCTCCC
CAAGTCCTCTGTGGCCAGCTGCATCTGTCTGAATGGTGCGTGAAGGCTC
TCAGACCTTACACACCATTTTGTAAGTTATGTTTTACATGCCCCGTTTT
TGAGACTGATCTCGATGCAGGTGGATCTCCTTGAGATCCTGATAGCCTG
TTACAGGAATGAAGTAAAGGTCAGTTTTTTTTGTATTGATTTTCACAGC
TTTGAGGAACATGCATAAGAAATGTAGCTGAAGTAGAGGGGACGTGAGA
GAAGGGCCAGGCCGGCAGGCCAACCCTCCTCCAATGGAAATTCCCGTGT
TGCTTCAAACTGAGACAGATGGGACTTAACAGGCAATGGGGTCCACTTC
CCCCTCTTCAGCATCCCCCGTACCCCACTTTTTGCTGAAAGAACTGCCA
GCAGGTAGGACCCCAGAGGCCCCCAAATGAAAGCTTGAATTTCCCCTAC
TGGCTCTGCGTTTTGCTGAGATCTGTAGGAAAGGATGCTTCACAAACTG
AGGTAGATAATGCTATGCTGTCGTTGGTATACATCATGAATTTTTATGT
AAATTGCTCTGCAAAGCAAATTGATATGTTTGATAAATTTATGTTTTTA
GGTAAATAAAAACTTTTAAAAAGTTGTT
SEQ ID NO: 29
CCACAGAUCUAUCCGCCAU
SEQ ID NO: 30
UGUCAGAGCUGCAAGCGUU
SEQ ID NO: 31
GAAAAUGGUUGCCAGGUGA

Publications cited herein are hereby specifically incorporated by reference in their entireties and at least for the material for which they are cited.

It should be understood that while the present disclosure has been provided in detail with respect to certain illustrative and specific aspects thereof, it should not be considered limited to such, as numerous modifications are possible without departing from the broad spirit and scope of the present disclosure as defined in the appended claims. It is, therefore, intended that the appended claims cover all such equivalent variations as fall within the true spirit and scope of the invention.

1. A method for improving a transplant outcome in a recipient of a transplant comprising inhibiting IRF4 in T-cells of the recipient, thereby improving the transplant outcome.

2. The method of claim 1, wherein IRF4 is inhibited by administering to the recipient an IRF4 inhibitor.

3. The method of claim 2, wherein the IRF4 inhibitor comprises a MEK 1/2 inhibitor.

4. The method of claim 2, wherein the IRF4 inhibitor comprises trametinib.

5. The method of claim 2, wherein the IRF4 inhibitor comprises an anti-IRF4 siRNA.

6. The method of claim 1, wherein inhibiting IRF4 comprises reducing IRF4 polypeptide expression by at least 50% compared to a control.

7. The method of claim 6, wherein the control comprises an unmodified T-cell of the recipient.

8. The method of claim 1, wherein the T-cells comprise activated T-cells.

9. The method of claim 1, wherein the T-cells comprise CD4+ T-cells.

10. The method of claim 1, wherein the recipient comprises a MHC profile which is fully mismatched compared to a donor of the transplant.

11. The method of claim 1, wherein the improved transplant outcome comprises reduced inflammation in the transplant.

12. The method of claim 1, wherein the improved transplant outcome comprises reduced T-cell infiltration in the transplant.

13. The method of claim 1, wherein the improved transplant outcome comprises acceptance of the transplant by the recipient for at least 100 days.

14. The method of claim 1, wherein IRF4 is inhibited prior to transplantation.

15. A method of treating a subject with a myelination disorder comprising inhibiting IRF4.

16. The method of claim 15, wherein the myelination disorder comprises multiple sclerosis.

17. The method of claim 15, wherein the myelination disorder comprises encephalomyelitis.

18. A method of increasing T-cell dysfunction in a subject comprising inhibiting IRF4, wherein the subject has an autoimmune disease or is a recipient of a transplant.

19. A method to identify a compound which inhibits IRF4 comprising:
contacting one or more T-cell s with the compound; and
measuring IRF4 expression in the one or more T-cells;
wherein reduced IRF4 expression compared to a control indicates the compound inhibits IRF4; and wherein the control comprises one or more T-cells which are not contacted with the compound.

20. A method to measure IRF4 expression in T-cells of a subject prescribed to receive a transplant comprising:
obtaining T-cells from the subject; and
measuring IRF4 expression in the T-cells.
